(19) United States
(12) Patent Application Publication
(10) Pub. No.: US 2021/0215720 A1
(43) Pub. Date: Jul. 15, 2021

(54) PERSONALIZED MEDICINE APPROACH FOR TREATING COGNITIVE LOSS

(71) Applicant: University of North Texas Health Science Center at Fort Worth, Fort Worth, TX (US)

(72) Inventor: Sid E. O'Bryant, Aledo, TX (US)

(21) Appl. No.: 17/193,907

(22) Filed: Mar. 5, 2021

Related U.S. Application Data
(63) Continuation-in-part of application No. 15/037,492, filed on May 18, 2016.
(60) Provisional application No. 61/908,812, filed on Nov. 26, 2013.

(51) Int. Cl. G01N 33/68

(52) U.S. Cl. CPC G01N 33/6896; G01N 2800/60; G01N 2800/52

(57) ABSTRACT

The present disclosure relates to a method of treating a subject having a proinflammatory endophenotype profile with celecoxib or naproxen to improve cognition or to prevent cognitive decline or dysfunction in the subject. In another aspect, the present disclosure relates to a method of screening a subject for inclusion an NSAID or a PPAR-γ agonist clinical trial. In another aspect, the present disclosure relates to a method of determining a surrogate outcome of an NSAID or a PPAR-γ agonist clinical trial. In yet another aspect, the present disclosure relates to a method of treating an Alzheimer's disease patient having both a proinflammatory endophenotype profile and a metabolic endophenotype profile with a PPAR-γ agonist to improve cognition or to prevent cognitive decline or dysfunction in the patient.

### CROSS-REFERENCE TO RELATED APPLICATIONS

The present application is a continuation-in-part of, and claims priority to, U.S. patent application Ser. No. 15/037,492, filed May 18, 2016, which claims priority to, and is the National Phase of, International Application No. PCT/US2014/067562, filed Nov. 26, 2014, which claims priority under 35 U.S.C. § 119(e) to U.S. Provisional Application No. 61/908,812, filed Nov. 26, 2013, all of which applications are hereby incorporated by reference in their entireties.

### STATEMENT OF FEDERALLY FUNDED RESEARCH

This invention was made with Government support under AG051848 awarded by National Institutes of Health. The Government has certain rights in the invention.

### BACKGROUND OF THE INVENTION

Without limiting the scope of the invention, its background is described in connection with the treatment of cognitive dysfunctions. The detection and evaluation of disease conditions has progressed greatly as a result of the sequencing of the human genome and the availability of bioinformatics tools. One such system is taught in U.S. Pat. No. 8,430,816, for a system and method for analysis of multiple diseases and severities. Briefly, these inventors teach a data processing technique that includes a computer-implemented method for accessing reference deviation maps for a plurality of disease types. The reference deviation maps may include subsets of maps associated with severity levels of respective disease types and a disease severity score may be associated with each severity level. The method is said to also include selecting patient severity levels for multiple disease types based on the subsets of reference deviation maps. Also, the method may include automatically calculating a combined patient disease severity score based at least in part on the disease severity scores associated with the selected patient severity levels, and may include outputting a report based at least in part on the combined patient disease severity score.

Another such invention, is taught in U.S. Pat. No. 8,008,025, issued to Zhang and directed to biomarkers for neurodegenerative disorders. Briefly, this inventor teaches methods for diagnosing neurodegenerative disease, such as Alzheimer's Disease, Parkinson's Disease, and dementia with Lewy body disease by detecting a pattern of gene product expression in a cerebrospinal fluid sample and comparing the pattern of gene product expression from the sample to a library of gene product expression pattern known to be indicative of the presence or absence of a neurodegenerative disease. The methods are also said to provide for monitoring neurodegenerative disease progression and assessing the effects of therapeutic treatment. Also provided are kits, systems and devices for practicing the subject methods.

United States Patent Application Publication No. 2013/0012403, filed by Hu is directed to compositions and methods for identifying autism spectrum disorders. This application is directed to microRNA chips having a plurality of different oligonucleotides with specificity for genes associated with autism spectrum disorders. The invention is said to provide methods of identifying microRNA profiles for neurological and psychiatric conditions including autism spectrum disorders, methods of treating such conditions, and methods of identifying therapeutics for the treatment of such neurological and psychiatric conditions.

Yet another application is United States Patent Application Publication No. 2011/0159527, filed by Schlossmacher, et al., for methods and kits for diagnosing neurodegenerative disease. Briefly, the application is said to teach methods and diagnostic kits for determining whether a subject may develop or be diagnosed with a neurodegenerative disease. The method is said to include quantitating the amount of alpha-synuclein and total protein in a cerebrospinal fluid (CSF) sample obtained from the subject and calculating a ratio of alpha-synuclein to total protein content; comparing the ratio of alpha-synuclein to total protein content in the CSF sample with the alpha-synuclein to total protein content ratio in CSF samples obtained from healthy neurodegenerative disease-free subjects; and determining from the comparison whether the subject has a likelihood to develop neurodegenerative disease or making a diagnosis of neurodegenerative disease in a subject. It is said that a difference in the ratio of alpha-synuclein to total protein content indicates that the subject has a likelihood of developing a neurodegenerative disease or has developed a neurodegenerative disease.

### SUMMARY OF THE INVENTION

In one aspect, the present disclosure relates to a method of treating a subject to improve cognition or to prevent cognitive decline or dysfunction in the subject, the method comprising: obtaining a blood, plasma, or serum sample from the subject; measuring in the sample the expression level of one or more proinflammatory biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY); determining that the subject has a proinflammatory endophenotype profile based on the expression level of the proinflammatory biomarkers; and administering an NSAID selected from naproxen and celecoxib to the subject. In one embodiment, the subject is treated to improve cognition or to prevent cognitive decline or dysfunction related to memory loss, senility, dementia, Alzheimer's disease, or a combination thereof. In one embodiment, the proinflammatory endophenotype profile is determined by comparing the expression level of one or more biomarkers in the blood, plasma, or serum sample obtained from the subject to the average expression level of the one or more corresponding biomarkers from a statistical sample representative of the subject. In one embodiment, the method further comprises one of steps (a)-(d): (a) determining that the blood, plasma, or serum sample obtained from the subject has a statistically different expression level of each of IL-10, eotaxin 3, A2M, and SAA, and optionally Tenacin C when compared to a group of individuals in the statistical sample who do not have a family history of memory loss, senility, dementia, Alzheimer's disease, or a combination thereof; and administering celecoxib to the subject; (b) determining that the blood, plasma, or serum sample obtained from the subject has a statistically similar expression level of each of IL-10, eotaxin 3, A2M, and SAA, and optionally Tenacin C when compared to a group of individuals in the statistical sample who have a family history of memory loss, senility, dementia, Alzheimer's disease, or a combination thereof; and administering celecoxib to the subject; (c) determining that the blood, plasma, or serum sample obtained from the subject has a statistically different expression level of each of TPO, IL-5, IL-7, and I309, and optionally IL-6 when compared to a group of individuals in the statistical sample who do not have a family history of memory loss, senility, dementia, Alzheimer's disease, or a combination thereof; and administering naproxen to the subject; or (d) determining that the blood, plasma, or serum sample obtained from the subject has a statistically similar expression level of each of TPO, IL-5, IL-7, and I309, and optionally IL-6 when compared to a group of individuals in the statistical sample who have a family history of memory loss, senility, dementia, Alzheimer's disease, or a combination thereof; and administering naproxen to the subject. In one embodiment, the method further comprises at least one of (a) or (b): (a) obtaining a result of one or more neurocognitive evaluations from the subject before administration of the NSAID; obtaining the result of the corresponding one or more neurocognitive evaluations from the subject after administration of the NSAID; and comparing the results of the one or more neurocognitive evaluations before administration of the NSAID with those after administration of the NSAID; or (b) obtaining a blood, plasma, or serum sample from the subject after administration of the NSAID; measuring in the sample the expression level of the corresponding one or more proinflammatory biomarkers; and comparing the expression level of the one or more biomarkers in the blood, plasma, or serum sample before administration of the NSAID with those after administration of the NSAID. In one embodiment, the one or more neurocognitive evaluations are selected from the group consisting of a 4-point clock drawing test, a verbal fluency test, a trail making test, a list learning test, a mini-mental state exam, and combinations thereof. In one embodiment, the step of administering an NSAID selected from naproxen and celecoxib to the subject comprises administering between about 175-225 mg of naproxen or celecoxib to the subject between once and eight times a day.

In another aspect, the present disclosure relates to a method of determining a surrogate outcome of an NSAID clinical trial to improve cognition or to prevent cognitive decline or dysfunction, the method comprising: (a) selecting a subject to participate in the clinical trial, wherein the subject is suspected of having age-related memory loss, senility, dementia, or Alzheimer's Disease, or having an elevated risk of age-related memory loss, senility, dementia, or Alzheimer's Disease; (b) measuring in a blood, plasma, or serum sample obtained from the subject the expression level of one or more biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY); (c) administering an NSAID selected from celecoxib and naproxen to a first subset of subjects and a placebo to a second subset of subjects; (d) repeating step (b) after the administration of the NSAID or the placebo; and (e) determining if a statistically significant change in the expression level of the one or more biomarkers is seen in the first subset of subjects when compared to any change occurring in the second subset of subjects, wherein a statistically significant change in the expression level is associated with a beneficial long term clinical outcome. In one embodiment, when the NSAID administered in the clinical trial is celecoxib, the expression level of each of the biomarkers FABP, tenacin C, B2M, and TPO, and optionally IL-5 is measured in steps (b) and (d); and when the NSAID administered in the clinical trial is naproxen, the expression level of each of the biomarkers TPO, IL-6, CRP, and FABP, and optionally Factor VII is measured in steps (b) and (d).

In another aspect, the present disclosure relates to a method of treating an Alzheimer's disease patient to improve cognition or to prevent cognitive decline or dysfunction in the patient, the method comprising: obtaining a blood, plasma, or serum sample from the patient; measuring in the sample the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol; determining that the patient has both a proinflammatory endophenotype profile and a metabolic endophenotype profile based on the expression level of the one or more biomarkers; and administering a PPAR-γ agonist to the patient. In one embodiment, the patient is determined to have both a proinflammatory endophenotype profile and a metabolic endophenotype profile by comparing the expression level of the one or more biomarkers in the blood, plasma, or serum sample obtained from the patient to the average expression level of the one or more corresponding biomarkers from a statistical sample representative of the patient. In one embodiment, a statistically different expression level in the blood, plasma, or serum sample of one or more biomarkers selected from IL-6, IL-10, CRP, TNFα, FABP and PPY when compared to the expression level of the one or more corresponding biomarkers from individuals in the statistical sample who do not have Alzheimer's disease indicates that the patient has both a proinflammatory endophenotype profile and a metabolic endophenotype profile; or a statistically similar expression level in the blood, plasma, or serum sample of the one or more biomarkers selected from IL-6, IL-10, CRP, TNFα, FABP and PPY when compared to the expression level of the one or more corresponding biomarkers from individuals in the statistical sample who have been diagnosed with Alzheimer's disease indicates that the patient has both a proinflammatory endophenotype profile and a metabolic endophenotype profile. In one embodiment, a statistically different expression level in the blood, plasma, or serum sample of each of IL-6, IL-10, CRP, TNFα, FABP and PPY when compared to the expression level of each of the corresponding biomarkers from individuals in the statistical sample who do not have Alzheimer's disease indicates that the patient has both a proinflammatory endophenotype profile and a metabolic endophenotype profile; or a statistically similar expression level in the blood, plasma, or serum sample of each of IL-6, IL-10, CRP, TNFα, FABP and PPY when compared to the expression level of each of the corresponding biomarkers from individuals in the statistical sample who have been diagnosed with Alzheimer's disease indicates that the patient has both a proinflammatory endophenotype profile and a metabolic endophenotype profile. In one embodiment, the PPAR-γ agonist is selected from rosiglitazone and rosiglitazone XR. In one embodiment, the step of administering a PPAR-γ agonist to the patient comprises administering between about 2-10 mg of a PPAR-γ agonist is selected from rosiglitazone and rosiglitazone XR to the patient between once and eight times a day. In one embodiment, the method further comprises measuring a body mass index (BMI) and/or waist circumference of the patient; determining that the BMI and/or waist circumference of the patient is higher when compared to the average BMI and/or waist circumference from individuals in the statistical sample who do not have Alzheimer's disease, or determining that the BMI and/or waist circumference of the patient is higher when compared to the average BMI and/or waist circumference from individuals in the statistical sample who have been diagnosed with Alzheimer's disease; comparing the expression level of one or more biomarkers selected from the group consisting of IL-6, IL-10, CRP, TNFα, FABP, and PPY in the blood, plasma, or serum sample obtained from the patient to the average expression level of the one or more corresponding biomarkers from a statistical sample representative of the patient; and determining that the patient has both a proinflammatory endophenotype profile and a metabolic endophenotype profile. In one embodiment, the method further comprises at least one of (a) or (b): (a) obtaining a result of one or more neurocognitive evaluations from the patient before administration of a PPAR-γ agonist; obtaining the result of the corresponding one or more neurocognitive evaluations from the patient after administration of the PPAR-γ agonist; and comparing the results of the one or more neurocognitive evaluations before administration of the PPAR-γ agonist with those after administration of the PPAR-γ agonist; (b) obtaining a blood, plasma, or serum sample from the patient after administration of the PPAR-γ agonist; measuring in the sample the expression level of the corresponding one or more biomarkers; and comparing the expression level of the one or more biomarkers in the blood, plasma, or serum sample before administration of the PPAR-γ agonist with those after administration of the PPAR-γ agonist. In one embodiment, the one or more neurocognitive evaluations are selected from the group consisting of a 4-point clock drawing test, a verbal fluency test, a trail making test, a list learning test, a mini-mental state exam, and combinations thereof.

In yet another aspect, the present disclosure relates to a method of determining a surrogate outcome of a PPAR-γ agonist clinical trial to improve cognition or to prevent cognitive decline or dysfunction, the method comprising: (a) selecting a subject to participate in the clinical trial, wherein the subject is suspected of having age-related memory loss, senility, dementia, or Alzheimer's Disease, or having an elevated risk of age-related memory loss, senility, dementia, or Alzheimer's Disease; (b) measuring, in a blood, plasma, or serum sample obtained from the subject, the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol; (c) determining that the subject has both a proinflammatory endophenotype profile and a metabolic endophenotype profile based on the expression level of the one or more biomarkers; (d) administering a PPAR-γ agonist to a first subset of subjects and a placebo to a second subset of subjects; (e) repeating step (b) after the administration of the PPAR-γ agonist or the placebo; and (f) determining if a statistically significant change in the expression level of the one or more biomarkers is seen in the first subset of subjects when compared to any change occurring in the second subset of subjects, wherein a statistically significant change in the expression level is associated with a beneficial long term clinical outcome. In one embodiment, the expression level of each of from IL-6, IL-10, CRP, TNFα, FABP and PPY, and optionally the expression level of one or more of IL-7, IL-5, TNC, ICAM1, Factor VII, I309, TNFR1, A2M, TARC, eotaxin3, VCAM1, TPO, IL-18, B2M, SAA, DJ1, Aβ, tau, α-synuclein, GLP-1, PYY, insulin, HbA1c, glucose, triglycerides, HDL, LDL, vLDL, DGAT1, PPAR-γ, PPARα, and cholesterol, are measured in steps (b) and (e). In one embodiment, the PPAR-γ agonist is selected from rosiglitazone and rosiglitazone XR.

In yet another aspect, the present disclosure relates to a method of screening a subject for inclusion in an NSAID clinical trial to improve cognition or to prevent cognitive decline or dysfunction in the subject, the method comprising: selecting the subject for screening based on one or more demographic factors or a result of one or more neurocognitive evaluations; obtaining a blood, plasma, or serum sample from the subject; measuring in the sample the expression level of one or more proinflammatory biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY); determining that the subject has a proinflammatory endophenotype profile based on the expression level of the proinflammatory biomarkers; and selecting the subject for inclusion in the NSAID clinical trial. In one embodiment, the proinflammatory endophenotype profile is determined by comparing the expression level of one or more biomarkers in the blood, plasma, or serum sample obtained from the subject to the average expression level of the one or more corresponding biomarkers from a statistical sample representative of the subject. In one embodiment, the method further comprises one of steps (a)-(d): (a) determining that the blood, plasma, or serum sample obtained from the subject has a statistically different expression level of each of IL-10, eotaxin 3, A2M, and SAA, and optionally Tenacin C when compared to a group of individuals in the statistical sample who do not have Alzheimer's disease; and selecting the subject for a celecoxib clinical trial; (b) determining that the blood, plasma, or serum sample obtained from the subject has a statistically similar expression level of each of IL-10, eotaxin 3, A2M, and SAA, and optionally Tenacin C when compared to a group of individuals in the statistical sample who have Alzheimer's disease; and selecting the subject for a celecoxib clinical trial; (c) determining that the blood, plasma, or serum sample obtained from the subject has a statistically different expression level of each of TPO, IL-5, IL-7, and I309, and optionally IL-6 when compared to a group of individuals in the statistical sample who do not have Alzheimer's disease; and selecting the subject for a naproxen clinical trial; or (d) determining that the blood, plasma, or serum sample obtained from the subject has a statistically similar expression level of each of TPO, IL-5, IL-7, and I309, and optionally IL-6 when compared to a group of individuals in the statistical sample who have Alzheimer's disease; and selecting the subject for a naproxen clinical trial. In one embodiment, the one or more demographic factors are selected from the group consisting of age, education level, and APOE ε4 allele frequency; and the one or more neurocognitive evaluations are selected from the group consisting of a 4-point clock drawing test, a verbal fluency test, a trail making test, a list learning test, a mini-mental state exam, and combinations thereof.

In yet another aspect, the present disclosure relates to a method of screening a subject for inclusion in a PPAR-γ agonist clinical trial to improve cognition or to prevent cognitive decline or dysfunction in the subject, the method comprising: (a) selecting the subject for screening based on one or more demographic factors or a result of one or more neurocognitive evaluations; (b) obtaining a blood, plasma, or serum sample from the subject; (c) measuring in the sample the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol; (d) determining that the subject has both a proinflammatory endophenotype profile and a metabolic endophenotype profile based on the expression level of the one or more biomarkers; and (e) selecting the subject for inclusion in the PPAR-γ agonist clinical trial. In one embodiment, the subject is determined to have both a proinflammatory endophenotype profile and a metabolic endophenotype profile by comparing the expression level of the one or more biomarkers in the plasma or serum sample obtained from the subject to the average expression level of the one or more corresponding biomarkers from a statistical sample representative of the subject. In one embodiment, the expression level of each of from IL-6, IL-10, CRP, TNFα, FABP and PPY, and optionally the expression level of one or more of IL-7, IL-5, TNC, ICAM1, Factor VII, I309, TNFR1, A2M, TARC, eotaxin3, VCAM1, TPO, IL-18, B2M, SAA, DJ1, Aβ, tau, α-synuclein, GLP-1, PYY, insulin, HbA1c, glucose, triglycerides, HDL, LDL, vLDL, DGAT1, PPAR-γ, PPARα, and cholesterol, is measured in step (c). In one embodiment, the PPAR-γ agonist is selected from rosiglitazone and rosiglitazone XR. In one embodiment, the one or more demographic factors are selected from the group consisting of age, education level, APOE ε4 allele frequency, body mass index, waist circumference, and combinations thereof; and the one or more neurocognitive evaluations are selected from the group consisting of a 4-point clock drawing test, a verbal fluency test, a trail making test, a list learning test, a mini-mental state exam, and combinations thereof.

### BRIEF DESCRIPTION OF THE DRAWINGS

For a more complete understanding of the features and advantages of the present invention, reference is now made to the detailed description of the invention along with the accompanying figures and in which:

FIG. 1 shows three endophenotypes for cognitive loss.

FIG. 2 is a graph that shows the effect of treating subjects with naproxen and a placebo with three different pro-inflammatory endophenotypes.

FIG. 3 is a graph that shows the progression of disease when treating subjects with naproxen and a placebo with three different pro-inflammatory endophenotypes.

FIG. 4 is a graph that shows the linear decrease in cognitive functioning three different pro-inflammatory endophenotypes.

FIG. 5 shows the linear increase in disease severity on the pro-inflammatory endophenotypes among patients with Alzheimer's Disease.

FIG. 6 shows the linear decline in baseline cognitive ability among non-demented normal controls as a function of the pro-inflammatory endophenotypes.

FIG. 7 shows that global cognitive ability (MMSE scores) varies as a function of BDNF levels by patient type (1=Alzheimer's disease, 2=normal control, 3=mild cognitive impairment) for a neurotrophic endophenotype.

FIG. 8 shows a linear decrease in cognitive functioning (MMSE scores).

FIG. 9 demonstrates a linear increase in disease severity based on the pro-inflammatory profile among AD patients.

FIG. 10 demonstrates the same linear decline in baseline cognitive ability (MMSE scores) among non-demented normal controls as a function of the pro-inflammatory profile.

FIG. 11 is a graph that shows the results for the treatment group—(a) those in the low end of the pro-inflammatory profile (Group 1.00) who were treated with an anti-inflammatory drug declined significantly faster (i.e. disease severity and cognition) when compared to the referent group (i.e. middle group; Group 2.00)), (b) those in the high end (Group 3.00) were stable over 12 months when treated with an anti-inflammatory drug when compared to the low end of the pro-inflammatory profile and the referent group.

FIG. 12 is a graph that shows disease severity (i.e. CDR Sum of Boxes [CDRSum]) for the three Groups shown in FIG. 13 with treatment with an NAIDS (naproxen) or placebo.

FIG. 13 shows the baseline 20-protein predictive biomarker for predicting response in the Celecoxib arm from baseline to 12 months with corresponding receiver operating curve and Gini plot.

FIG. 14 is a chart of the baseline 20-protein predictive biomarker algorithm for detecting response in the Celecoxib arm from baseline to 12 months.

FIG. 15 shows the 12-month 20-protein predictive biomarker for predicting response in the Celecoxib arm from baseline to 12 months with corresponding receiver operating curve and Gini plot.

FIG. 16 is a chart of the 12-month 20-protein predictive biomarker algorithm for detecting response in the Celecoxib arm from baseline to 12 months.

FIG. 17 shows the baseline 20-protein predictive biomarker for predicting response in the Naproxen arm from baseline to 12 months with corresponding receiver operating curve and Gini plot.

FIG. 18 is a chart of the baseline 20-protein predictive biomarker algorithm for detecting response in the Naproxen arm from baseline to 12 months.

FIG. 19 shows the 12-month 20-protein predictive biomarker for predicting response in the Naproxen arm from baseline to 12 months with corresponding receiver operating curve and Gini plot.

FIG. 20 is a chart of the 12-month 20-protein predictive biomarker algorithm for detecting response in the Naproxen arm from baseline to 12 months.

FIG. 21 depicts the precision medicine approach to trial enrollment with predictive biomarkers.

FIG. 22 shows the predictive biomarker accuracy in predicting treatment response in the Phase 2 trial.

FIG. 23 shows the predictive biomarker accuracy in identifying responders versus non-responders in the Phase 3 trial AV105640.

FIG. 24 shows the predictive biomarker accuracy in identifying responders versus non-responders in the Phase 3 trial AV102672.

FIG. 25 shows the predictive biomarker accuracy in identifying responders versus non-responders in the Phase 3 trial AV102670.

FIG. 26 shows the predictive biomarker accuracy in identifying responders versus non-responders in across the 2 mg XR and 8 mg XR arms across trials.

FIG. 27 shows the predictive accuracy in identifying responders versus non-responders dosages.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the phrase “neurological disease” refers to a disease or disorder of the central nervous system and many include, e.g., neurodegenerative disorders such as AD, Parkinson's disease, mild cognitive impairment (MCI) and dementia and neurological diseases include multiple sclerosis, neuropathies. The present invention will find particular use in treating cognitive dysfunction associated with AD and other neurodegenerative disorders such as Parkinson's Disease, Frontotemporal dementia, Dementia with Lewy Bodies, and Down's syndrome.

As used herein, the terms “Alzheimer's patient”, “AD patient”, and “individual diagnosed with AD” all refer to an individual who has been diagnosed with AD or has been given a probable diagnosis of Alzheimer's Disease (AD).

As used herein, the terms “Parkinson's disease patient”, and “individual diagnosed with Parkinson's disease” all refer to an individual who has been diagnosed with PD or has been given a diagnosis of Parkinson's disease.

As used herein, the terms “Frontotemporal dementia”, and “individual diagnosed with frontotemporal dementia” all refer to an individual who has been diagnosed with FTD or has been given a diagnosis of FTD.

As used herein, the term “Dementia with Lewy bodies” (DLB), and “individual diagnosed with DLB” all refer to an individual who has been diagnosed with DLB or has been given a diagnosis of DLB.

As used herein, the term “Down's syndrome” (DS), and “individual diagnosed with Down's syndrome” all refer to an individual who has been diagnosed with DS or has been given a diagnosis of DS.

As used herein, the phrase “neurological disease biomarker” refers to a biomarker that is a neurological disease diagnosis biomarker.

As used herein, the term “neurological disease biomarker protein”, refers to any of: a protein biomarkers or substances that are functionally at the level of a protein biomarker.

As used herein, the terms “cognition”, “cognitive ability”, “memory”, “language” and the like are used interchangeably to refer to an individual's ability to perform cognitive abilities and the dysfunction of those abilities that may be as a result of a diagnosis of MCI, AD, DLB, FTD, DLB, Multiple Sclerosis (MS), PD, or other neurological disease as well as other medical and psychiatric conditions including, but not limited to, diabetes, hypertension, dyslipidemia, metabolic syndrome, depression, traumatic brain injury, schizophrenia, bipolar disease, as well as the cognitive slowing/decline associated with the aging process itself.

As used herein, methods for “aiding treatment” refer to methods that assist in making a clinical determination regarding the course of treatment of cognitive dysfunction associated with the neurological disease (e.g., AD, PD, DLB, FTD, DS or MCI), and may or may not be conclusive with respect to the definitive diagnosis.

As used herein, the term “stratifying” refers to sorting individuals into different classes or strata based on the features of a neurological disease. For example, stratifying a population of individuals with Alzheimer's disease involves assigning the individuals on the basis of the severity of the disease (e.g., mild, moderate, advanced, etc.).

As used herein, the term “predicting” refers to making a finding that an individual has a significantly enhanced probability of developing a certain neurological disease.

As used herein, “biological fluid sample” refers to a wide variety of fluid sample types obtained from an individual and can be used in a diagnostic or monitoring assay. Biological fluid sample include, e.g., blood, cerebral spinal fluid (CSF), urine and other liquid samples of biological origin. Commonly, the samples are treatment with stabilizing reagents, solubilization, or enrichment for certain components, such as proteins or polynucleotides, so long as they do not interfere with the analysis of the markers in the sample.

As used herein, a “blood sample” refers to a biological sample derived from blood, preferably peripheral (or circulating) blood. A blood sample may be, e.g., whole blood, serum or plasma. In certain embodiments, serum is preferred as the source for the biomarkers as the samples are readily available and often obtained for other sampling, is stable, and requires less processing, thus making it ideal for locations with little to refrigeration or electricity, is easily transportable, and is commonly handled by medical support staff.

As used herein, a “normal” individual or a sample from a “normal” individual refers to quantitative data, qualitative data, or both from an individual who has or would be assessed by a physician as not having a disease, e.g., a neurological disease. Often, a “normal” individual is also age-matched within a range of 1, 2, 3, 4, 5, 6, 7, 8, 9 or 10 years with the sample of the individual to be assessed.

As used herein, the term “treatment” refers to the alleviation, amelioration, and/or stabilization of symptoms, as well as delay in progression of symptoms of a particular disorder. For example, “treatment” of AD includes any one or more of: (1) elimination of one or more symptoms of AD, (2) reduction of one or more symptoms of AD, (4) stabilization of the symptoms of AD (e.g., failure to progress to more advanced stages of AD), and (5) delay in onset of one or more symptoms of AD delay in progression (i.e., worsening) of one or more symptoms of AD; and (6) delay in progression (i.e., worsening) of one or more symptoms of AD.

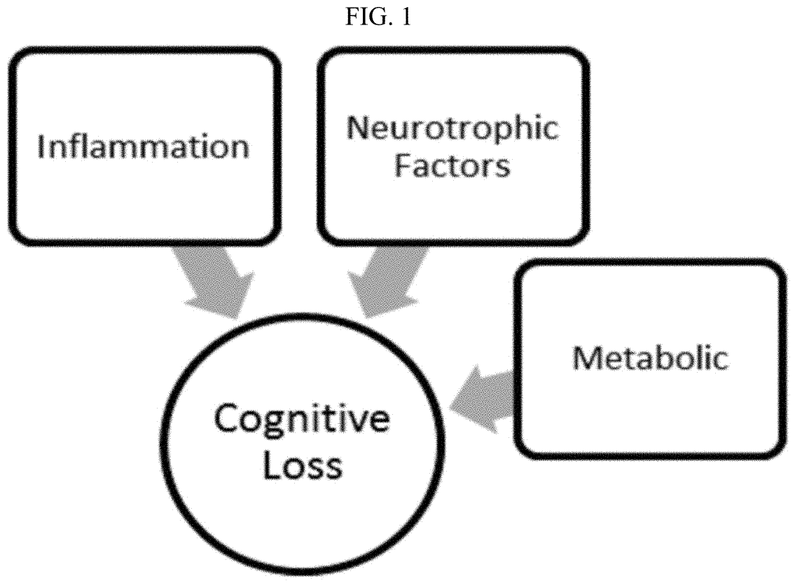

As used herein, the term “endophenotype” refers to a subgroup of patients within a broader category, which can be defined by biological, cognitive, or psychological/questionnaire data. FIG. 1 shows three endophenotypes for cognitive loss. For example, within patients diagnosed with traumatic brain injury (TBI) who are suffering from cognitive loss, those TBI patients may be subdivided into groups based on a pro-inflammatory endophenotype, neurotrophic factor endophenotype, and metabolic endophenotype.

As used herein, the term “fold difference” refers to a numerical representation of the magnitude difference between a measured value and a reference value, e.g., an AD biomarker, a Parkinson's biomarker, a dementia biomarker, or values that allow for the differentiation of one or more of the neurological diseases. Typically, fold difference is calculated mathematically by division of the numeric measured value with the numeric reference value. For example, if a measured value for an AD biomarker is 20 nanograms/milliliter (ng/ml), and the reference value is 10 ng/ml, the fold difference is 2 (20/10=2). Alternatively, if a measured value for an AD biomarker is 10 nanograms/milliliter (ng/ml), and the reference value is 20 ng/ml, the fold difference is 10/20 or −0.50 or −50%).

As used herein, a “reference value” can be an absolute value, a relative value, a value that has an upper and/or lower limit, a range of values; an average value, a median value, a mean value, or a value as compared to a particular control or baseline value. Generally, a reference value is based on an individual sample value, such as for example, a value obtained from a sample from the individual with e.g., a neurological disease such as AD, Parkinson's Disease, or dementia, preferably at an earlier point in time, or a value obtained from a sample from an neurological disease patient other than the individual being tested, or a “normal” individual, that is an individual not diagnosed with AD, Parkinson's Disease, or dementia. The reference value can be based on a large number of samples, such as from AD patients, Parkinson's Disease patients, dementia patients, or normal individuals or based on a pool of samples including or excluding the sample to be tested.

As used herein, the phrase “a pre-determined amount of time” is used to describe the length of time between measurements that would yield a statistically significant result, which in the case of disease progression for cognitive loss can be 7 days, 2 weeks, one month, 3 months, 6 months, 9 months, 1 year, 1 year 3 months, 1 year 6 months, 1 year 9 months, 2 years, 2 years 3 months, 2 years 6 months, 2 years 9 months, 3, 4, 5, 6, 7, 8, 9 or even 10 years and combinations thereof.

As used herein, the phrases “neurocognitive screening tests,” “neurocognitive evaluations,” or “cognitive test” are used to describe one or more tests known to the skilled artisan for measuring cognitive status or impairment and can include but is not limited to: a 4-point clock drawing test, an verbal fluency test, trail making test, list learning test, the Mini-Mental State Exam (MMSE), and the like. The skilled artisan will recognize and know how these tests can be modified, how new tests that measure similar cognitive function can be developed and implemented for use with the present invention.

As used herein, intracellular adhesion molecule-1 (ICAM1 or ICAM-1) is interchangeable with soluble intracellular adhesion molecule-1 (sICAM1 or sICAM-1).

As used herein, vascular cell adhesion molecule-1 (VCAM1 or VCAM-1) is interchangeable with soluble vascular cell adhesion molecule-1 (sVCAM1 or sVCAM-1).

As used herein, FABP and FABP3 are used interchangeably to refer to fatty acid binding protein.

As used herein a “statistical sample representative of the subject” or a “statistical sample representative of the patient” refers to a statistical sample comprising one or more of the following groups of individuals:

1) individuals with a family history of memory loss, senility, dementia, or Alzheimer's disease;
2) individuals with no family history of memory loss, senility, dementia, or Alzheimer's disease;
3) individuals who have Alzheimer's disease; and
4) individuals who do not have Alzheimer's disease.

In some embodiments, the statistical sample comprises elderly individuals. In some embodiments, the elderly individuals are 50 years of age or older. In some embodiments, the data obtained from the patient or subject (e.g. demographic factors, neurocognitive evaluation results, and biomarker expression level) are compared to the corresponding data from individuals in the statistical sample. This comparison is discussed elsewhere herein and can be applied to any method of the present disclosure.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Cognitive loss is common among the aging/elderly population. Approximately 10-12% of all individuals age 65 and above suffer from Alzheimer's disease with another approximately 20% suffering from mild cognitive impairment (MCI), which is a prodromal phase to Alzheimer's disease. Additionally, cognitive loss is commonly associated with other neurodegenerative (e.g. Parkinson's disease, frontotemporal dementia), neurological (e.g. traumatic brain injury, multiple sclerosis), psychiatric (e.g. depression, bipolar, schizophrenia) and other medical conditions (e.g. diabetes, hypertension, dyslipidemia). On the other hand, the “one-size-fits-all” approach to treating cognitive loss among adults and elders has largely been a failure. For example, all clinical trials focusing on beta amyloid protein within Alzheimer's disease have failed in Phase III testing with no new medications approved for this disease in decades. Additionally, while there are well-established depression—cognition and diabetes—cognition links, trials focusing on disease specific interventions have been of limited benefit. These failures led to the present discovery, namely, that there are many underlying biological reasons for cognitive loss and that these systems may be largely “disease” irrelevant. For example, inflammation is related to many diseases (e.g. Alzheimer's disease, Parkinson's disease, cancer, multiple sclerosis, diabetes, TBI), which the present inventors have recognized are linked to poorer cognition across diseases. Therefore, the present inventors have subgrouped patients who are at increased risk for cognitive loss related to underlying dysfunction of the inflammatory, and other systems, and to treatment regimens that improve or prevent such cognitive loss across disease conditions. To date, there have been no strategies for prevention of cognitive loss that have been proven effective.

The present inventors have developed an endophenotype approach to treating and preventing cognitive loss among aging population. The term endophenotype¹ has been discussed frequently in psychiatry and they provide a way for identifying subgroups of clinical phenotypes². The present invention demonstrates three distinct endophenotypes that can be used to guide cognitive impairment therapy: inflammatory^{3,4}, neurotrophic factor⁵, and metabolic⁷ endophenotypes of cognitive loss. Endophenotypes of cognitive loss have also been identified based on neuropathology⁸, neuroimaging^{9,10}, genetics¹¹, and cerebrospinal fluid markers¹². The inventors provide herein four endophenotypes specifically designed to guide therapy and exemplary therapies for use with the invention.

### Pro-Inflammatory Endophenotype.

When providing treatment for those subjects identified with the pro-inflammatory endophenotypes, the treatment can include the following. Nonsteroidal anti-inflammatory drugs (NSAIDs): Non-selective NSAIDs—non-selective NSAIDs would be selected for those patients falling into the high end of the proinflammatory endophenotype. As shown herein, non-selective NSAIDs (naproxen) were the superior treatment to selective NSAIDs (celecoxib). Non-selective NSAIDs can be tested with anyone falling within the high end of the proinflammatory endophenotype.

Selective NSAIDs: selective NSAIDs (e.g. celecoxib) can be tested with those falling within the high end of the proinflammatory endophenotype.

Steroids: Many steroids, glucocorticoids, have anti-inflammatory properties and can be considered for those patients falling within the high end of the pro-inflammatory endophenotype.

Immune Selective Anti-Inflammatory Derivatives (ImSAIDs): ImSAIDs can be considered for patients falling within the high end of the proinflammatory endophenotype.

Anti-TNF medications can be specifically utilized for those within the high end of the proinflammatory endophenotype where TNFα weighs most heavily.

Anti-IL5 drugs can be utilized for those within the high end of the proinflammatory endophenotype where IL-5 weighs most heavily.

CRP-lowering agents can be selectively utilized for those in the high end of the proinflammatory endophenotype where CRP weighs most heavily.

### Metabolic Endophenotype.

When providing treatment for those subjects identified with the metabolic endophenotypes anti-diabetic medications can be utilized for those falling within the low or high end of the metabolic endophenotype, depending on the mechanism of action of the drug.

Insulin would be utilized for those whose metabolic endophenotype weighs insulin heavily. Insulin may be utilized also for those whose metabolic endophenotype weighs glucose levels most heavily.

GLP-1 medications would be utilized for those whose metabolic endophenotype weighs GLP-1 most heavily. In the inventors' prior work, GLP-1 was higher among those with cognitive dysfunction; however, higher levels of GLP-1 was associated with better memory and therefore would be administered for treatment of cognitive problems among those with cognitive loss and prevention of cognitive loss among cognitively normal elders.

Amylin-related medications can be utilized for those whose metabolic endophenotype weighs amylin most heavily.

Oral hypoglycemics can be tested among any patients who are in the high end of the metabolic endophenotype.

### Neurotrophic Endophenotype.

When providing treatment for those subjects identified with the neurotrophic endophenotypes neurotrophic factor agonists can be examined for improved cognitive function and prevention of cognitive loss among those in the low end of the neurotrophic endophenotype. Neurotrophic factor agonists can be examined for cognitive improvement among those in the high end of the neurotrophic endophenotype. It is unlikely that those in the middle group of the neurotrophic endophenotype will experience cognitive benefit or decline from such treatments.

Exercise therapy can be prescribed to any patients who fall into the low end of the neurotrophic endophenotype for prevention or treatment of cognitive loss as well as for improvement of cognitive loss among those in the high end of the neurotrophic endophenotype.

BDNF and BDNF agonists would be utilized for those patients in the low end of the neurotrophic endophenotype for improved cognition as well as prevention of cognitive loss. Such medications would be utilized for treating cognitive loss among the high end of the endophenotype. Selective serotonin reuptake inhibitors, selective serotonin 2C (5-HT2C) antagonists, serotonin-norepinephrine reuptake inhibitors, and tricyclic antidepressants have been found to increase BDNF levels and may be particularly useful in treating and/or preventing cognitive loss for those whose neurotrophic endophenotype weighs BDNF most heavily.

Combined exercise and medications such as selective serotonin reuptake inhibitors, selective serotonin 2C (5-HT2C) antagonists, serotonin-norepinephrine reuptake inhibitors, and tricyclic antidepressants have been found to increase BDNF levels and may be particularly useful in treating and/or preventing cognitive loss for those whose neurotrophic endophenotype weighs BDNF most heavily.

GDNF and GDNF agonists would be utilized for those patients in the low end of the neurotrophic endophenotype for improved cognition as well as prevention of cognitive loss. Such medications would be utilized for treating cognitive loss among the high end of the endophenotype.

### Combination Endophenotype.

In some embodiments, the subject has an endophenotype which is a combination of two or more of a proinflammatory endophenotype, a metabolic endophenotype, and a neurotrophic endophenotype. In one embodiment, the subject has a combination of a proinflammatory endophenotype and a metabolic endophenotype. In some embodiments, a subject having an endophenotype which is a combination of proinflammatory and metabolic is treated with a peroxisome proliferator-activated receptor-γ (PPAR-γ) agonist to treat and/or prevent cognitive loss. In some embodiments, the cognitive loss is cognitive loss associated with Alzheimer's disease. The PPAR-γ agonist can be any PPAR-γ agonist known to a person of skill in the art. In one embodiment, the PPAR-γ agonist is a thiazolidinedione. In one embodiment, the PPAR-γ is rosiglitazone or rosiglitazone XR.

### Methods

In one embodiment, the present invention includes a method for selecting a therapy for improved cognition or prevention of cognitive loss using one or more anti-inflammatory therapies comprising: obtaining a sample from a subject; measuring one or more biomarkers in the sample selected from at least one of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (FVII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, or α-synuclein; comparing the level of the one or more biomarkers within a sample of patients suffering from cognitive loss; dividing the level of expression of the one or more markers as being either high proinflammatory or low proinflammatory; and selecting a course of treatment for the subject based on whether the subject is selected as being high proinflammatory or low proinflammatory. In one aspect, the method further comprises the steps of: generating a high and a low proinflammatory endophenotype by determining the level of expression of two or more markers selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, Aβ, tau, or α-synuclein; and determining the high and low proinflammatory groupings by determining the level of expression of the two or more biomarkers. In another aspect, the proinflammatory profile is generated using learning machines (random forest, support vector machines), clustering algorithms (factor analysis, principal component analysis), summation of values, or other methods to generate a proinflammatory score across multiple measures. In another aspect, the high end of the score across multiple markers is reflective of the high proinflammatory endophenotype and the low end as the low proinflammatory endophenotype with all others falling in a middle endophenotype. In another aspect, if the subject is scored in the high proinflammatory group an anti-inflammatory treatment is indicated, and if the subject is scored in a low proinflammatory group then an anti-inflammatory treatment is contraindicated. In another aspect, at least one of the biomarker measurements is obtained by a method selected from the group consisting of immunoassay and enzymatic activity assay. In another aspect, the sample is serum or plasma. In another aspect, the cognitive dysfunction is a disease or condition selected from Alzheimer's Disease, Parkinson's Disease, Down's syndrome, Frontotemporal dementia, Dementia with Lewy Bodies, Multiple sclerosis, traumatic brain injury, depression, schizophrenia, bipolar disease (and other mental illness), diabetes, hypertension, stroke, heart attack, dyslipidemia, other conditions/diseases or aging. In another aspect, cognition is “normal” but patients are deemed “at risk” based on their pro-inflammatory endophenotype. In another aspect, the level of expression of the various proteins is measured by at least one of fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In another aspect, the high and low end of the proinflammatory group is determined by specifically determining the level of expression of C-reactive protein (CRP) and tumor necrosis factor alpha (TNFα). In another aspect, the high and low end of the proinflammatory group is determined from 2, 3, 4, 5, 6, 7, 8, 9, 10, 11, 12, 13, 14, 15, 16, 17, 18, 19, 20, 21, 22, 23, 24, or 25 biomarkers. In another aspect, the proinflammatory endophenotypes may be treated with one of more of the following non-limiting examples of therapeutic agents: NSAIDs, non-selective NSAIDs, selective NSAIDs, steroids, glucocorticoids, Immune Selective Anti-Inflammatory Derivatives (ImSAIDs), anti-TNF medications, anti-IL5 drugs, or CRP-lowering agents. In another aspect, the one or more of the following therapeutic agents: NSAIDs, non-selective NSAIDs, selective NSAIDs, steroids, glucocorticoids, Immune Selective Anti-Inflammatory Derivatives (ImSAIDs), anti-TNF medications, anti-IL5 drugs or CRP-lowering agents; are contraindicated if the subject does not have a proinflammatory endophenotype. In another aspect, the method further comprises the step of generating a dataset that comprises the level of the one or more biomarkers prior to the step of comparing the level of the one or more biomarkers within a sample of patients suffering from cognitive loss.

In another embodiment, the present invention includes a method for selecting patient therapy for improved cognition comprising: obtaining a sample from a subject; measuring the level of expression of TNFα and CRP in the sample; determining the tertile of the level of expression of the these two biomarkers; and depending on the level of expression dividing the level of expression of the two or more markers as being either high proinflammatory or low proinflammatory; and selecting a course of treatment for the subject based on whether the subject is selected as being high proinflammatory or low proinflammatory, wherein the tertile is determined by: scoring the tertile scores for both markers to generate a score with a range from two to six, assigning a lower score (i.e., 2) to the low end of a proinflammatory, assigning a highest score (i.e., 6) score was assigned to a high end of the proinflammatory, with all other scores falling in a middle score. In another aspect, the method further comprises the step of generating a dataset that comprises the level of expression of TNFα and CRP in the sample prior to the step of determining the tertile of the level of expression of the these two biomarkers; and depending on the level of expression dividing the level of expression of the two or more markers as being either high proinflammatory or low proinflammatory.

In another embodiment, the present invention includes a method for selecting patient therapy for prevention of cognitive loss comprising: obtaining a sample from a subject; measuring the level of expression of TNFα and CRP; determining the tertile of the level of expression of the these two biomarkers; and depending on the level of expression dividing the level of expression of the two or more markers as being either high proinflammatory or low proinflammatory; and selecting a course of treatment for the subject based on whether the subject is selected as being high proinflammatory or low proinflammatory, wherein the tertile is determined by: scoring the tertile scores for both markers to generate a score with a range from two to six, assigning a lower score (i.e., 2) to the low end of a proinflammatory, assigning a highest score (i.e., 6) score was assigned to a high end of the proinflammatory, with all other scores falling in a middle score. In another aspect, if the subject is scored in the tertile that is scored as a high proinflammatory an anti-inflammatory treatment is indicated, and if the subject is scored in a low proinflammatory then an anti-inflammatory treatment is contraindicated. In another aspect, the sample is serum or plasma. In another aspect, the cognitive dysfunction is a disease or condition selected from Alzheimer's Disease, Parkinson's Disease, Down's syndrome, Frontotemporal dementia, Dementia with Lewy Bodies, Multiple sclerosis, traumatic brain injury, depression, schizophrenia, bipolar disease (and other mental illness), diabetes, hypertension, stroke, heart attack, dyslipidemia, other conditions/diseases or aging. In another aspect, the level of expression of the various proteins is measured by at least one of fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling.

In another embodiment, the present invention includes a method determining the effectiveness of a candidate drug that impacts the inflammatory system to evaluate the candidate drug believed to be useful in treating a cognitive loss, the method comprising: (a) measuring one or more biomarkers in a sample of serum or plasma obtained from a subject suspected of having cognitive loss selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, Aβ, tau, or α-synuclein; (b) administering the candidate drug to a first subset of the patients, and a placebo to a second subset of the patients; (c) generating a proinflammatory group dataset using one or a combination of the one or more biomarkers; (d) determining the tertile of the level of expression of the one or more biomarkers; and depending on the level of expression dividing the level of expression of the one or more biomarkers as being either high proinflammatory or low proinflammatory; (e) determining if a baseline proinflammatory group predicted treatment response such that the high proinflammatory group responded differentially than the low proinflammatory group; (f) repeating step (a) after the administration of the candidate drug or the placebo; (g) determining if the candidate drug modifies the proinflammatory profile over the course of the trial; and (h) determining if change in the proinflammatory profile over the course of the trial predicted a positive response, a negative response, or a no treatment response, and if a statistically significant treatment response for cognitive loss, cognitive improvement or stability of cognitive functioning with the candidate drug is obtained, wherein a change in the proinflammatory profile is indicative of the candidate drug having effectiveness. In one aspect, the method further comprises the steps of obtaining one or more additional blood samples from the patient after a pre-determined amount of time and comparing the levels of the biomarkers from the one or more additional samples to determine progression of cognitive loss. In another aspect, the method further comprises the steps of treating the patient for a pre-determined period of time, obtaining one or more additional blood samples from the patient after the pre-determined amount of time and comparing the levels of the biomarkers from the one or more additional samples to determine progression of cognitive loss.

In another embodiment, the present invention includes a method of determining the effectiveness of a candidate drug that impacts the inflammatory system to evaluate the candidate drug believed to be useful in preventing cognitive loss, the method comprising: (a) measuring one or more biomarkers in a sample of serum or plasma obtained from a subject suspected of having cognitive loss selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, Aβ, tau, or α-synuclein; (b) administering the candidate drug to a first subset of the patients, and a placebo to a second subset of the patients; (c) generating the proinflammatory groups using one or a combination of the one or more biomarkers; (d) determining the tertile of the level of expression of the one or more biomarkers; and depending on the level of expression dividing the level of expression of the one or more markers as being either high proinflammatory or low proinflammatory; (e) determining if baseline proinflammatory group predicted treatment response such that the high proinflammatory group responded differentially than the low proinflammatory group; (f) repeating step (a) after the administration of the candidate drug or the placebo; (g) determining if the candidate drug modifies the proinflammatory profile over the course of the trial; and (h) determining if change in the proinflammatory profile over the course of the trial predicted a positive response, a negative response, or a no treatment response, and if a statistically significant treatment response for cognitive loss, cognitive improvement or stability of cognitive functioning with the candidate drug is obtained, wherein a change in the metabolic profile is indicative of the candidate drug having effectiveness. In one aspect, the method further comprises the steps of obtaining one or more additional blood samples from the patient after a pre-determined amount of time and comparing the levels of the biomarkers from the one or more additional samples to determine progression of cognitive loss. In another aspect, the method further comprises the steps of treating the patient for a pre-determined period of time, obtaining one or more additional blood samples from the patient after the pre-determined amount of time and comparing the levels of the biomarkers from the one or more additional samples to determine progression of cognitive loss.

In another embodiment, the present invention includes a method determining the effectiveness of a candidate drug that impacts the inflammatory system to evaluate the candidate drug believed to be useful in preventing or treating a cognitive loss, the method comprising: (a) measuring the serum or plasma based levels of CRP and TNFα; (b) administering the candidate drug to a first subset of the patients, and a placebo to a second subset of the patients; (c) generating the proinflammatory groups using a combination of CRP and TNFα for the first and second subset of patients; (d) determining the tertile of the level of expression of CRP and TNFα in the first and second subset of patients; (e) dividing the level of expression of CRP and TNFα as being either high proinflammatory or low proinflammatory depending on the level of expression of CRP and TNFα; (f) determining if baseline proinflammatory group predicted treatment response such that the high proinflammatory group responded differentially than the low proinflammatory group, (g) repeating step (a) after the administration of the candidate drug or the placebo; and (h) determining if the candidate drug modifies the proinflammatory profile over the course of the trial. In one aspect, the method further comprises the step of determining if change in the proinflammatory profile based on CRP and TNFα over the course of the trial predicted both positive and negative treatment response as well as no treatment response and if a statistically significant treatment response for the candidate drug was achieved as a primary or secondary outcome of the clinical trial. In another aspect, the method further comprises the steps of obtaining one or more additional blood samples from the patient after a pre-determined amount of time and comparing the levels of CRP and TNFα from the one or more additional samples to determine progression of cognitive loss. In another aspect, the method further comprises the steps of: treating the patient for a pre-determined period of time, obtaining one or more additional blood samples from the patient after the pre-determined amount of time and comparing the levels of the biomarkers from the one or more additional samples to determine progression of cognitive loss.

In another embodiment, the present invention includes a method for selecting a therapy for improved cognition using one or more anti-diabetic therapies comprising: obtaining a sample from a subject; measuring one or more biomarkers in the sample selected from alpha-2-macroglobulin (A2M), fatty acid binding protein (FABP), pancreatic polypeptide (PPP), glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, cholesterol, body mass index (BMI), or waist circumference; comparing the level of the one or more biomarkers within a sample of patients suffering from cognitive loss; dividing the level of expression of the one or more markers as being either high metabolic dysfunction endophenotype or low metabolic dysfunction endophenotype; and selecting a course of treatment for the subject based on whether the subject is selected as being high metabolic dysfunction endophenotype or low metabolic dysfunction endophenotype, wherein a high metabolic endophenotype subject benefits from a treatment with an anti-diabetic drug. In another aspect, the method further comprises the steps of: generating a high and a low metabolic endophenotype by determining the level of expression of two or more biomarkers selected from alpha-2-macroglobulin (A2M), fatty acid binding protein (FABP), pancreatic polypeptide (PPP), glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, cholesterol, body mass index (BMI), or waist circumference; and determining the high and low metabolic groupings by determining the level of expression of the two or more biomarkers. In one aspect, the metabolic profile is generated using learning machines (random forest, support vector machines), clustering algorithms (factor analysis, principal component analysis), summation of values, or other methods to generate a metabolic score across multiple measures. In another aspect, the high end of the score across multiple markers is reflective of the high metabolic dysfunction endophenotype and the low end as the low metabolic dysfunction endophenotype with all others falling in a middle endophenotype. In another aspect, if the subject is scored in the high metabolic dysfunction group an anti-diabetic treatment is indicated, and if the subject is scored in a low metabolic dysfunction group then an anti-diabetic treatment is contraindicated. In another aspect, at least one of the biomarker measurements is obtained by a method selected from the group consisting of immunoassay and enzymatic activity assay. In another aspect, the sample is serum or plasma.

In another aspect, the cognitive dysfunction is a disease or condition selected from Alzheimer's Disease, Parkinson's Disease, Down's syndrome, Frontotemporal dementia, Dementia with Lewy Bodies, Multiple sclerosis, traumatic brain injury, depression, schizophrenia, bipolar disease (and other mental illness), diabetes, hypertension, stroke, heart attack, dyslipidemia, other conditions/diseases or aging. In another aspect, cognition is “normal” but patients are deemed “at risk” based on their metabolic endophenotype. In another aspect, the level of expression of the various proteins is measured by at least one of fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In another aspect, the high and low end of the metabolic group is determined by specifically determining the level of expression of FABP and PPP. In another aspect, the high and low end of the metabolic group is determined from 2, 3, 4, 5, 6, 7, 8, 9, 10, 11, 12, 13, 14, 15, 16, 17, 18 or 19 biomarkers. In another aspect, the metabolic endophenotype may be treated with one of more of the following non-limiting examples of therapeutic agents: with anti-diabetic, insulin, GLP-1 medications would be utilized for those whose metabolic endophenotype weighs GLP-1, Amylin-related medications, or oral hypoglycemics.

In another embodiment, the present invention includes a method for selecting patient therapy for improved cognition or prevention of cognitive loss comprising: obtaining a sample from a subject; measuring the level of expression of two or more biomarkers selected from FABP and PPP; determining the tertile of the level of expression of the two or more biomarkers; and depending on the level of expression dividing the level of expression of the two or more markers as being either high metabolic or low metabolic; and selecting a course of treatment for the subject based on whether the subject is selected as being high metabolic endophenotype or low metabolic endophenotype, wherein the tertile is determined by: scoring the tertile scores for both markers to generate a score with a range from two to six, assigning a lower score (i.e., 2) to the low end of a metabolic, assigning a highest score (i.e., 6) score was assigned to a high end of the metabolic, with all other scores falling in a middle score. In one aspect, if the subject is scored in the tertile that is scored as a high metabolic an anti-diabetic treatment is indicated, and if the subject is scored in a low metabolic then an anti-diabetic treatment is contraindicated. In another aspect, the sample is serum or plasma. In another aspect, the cognitive dysfunction is a disease or condition selected from Alzheimer's Disease, Parkinson's Disease, Down's syndrome, Frontotemporal dementia, Dementia with Lewy Bodies, Multiple sclerosis, traumatic brain injury, depression, schizophrenia, bipolar disease (and other mental illness), diabetes, hypertension, stroke, heart attack, dyslipidemia, other conditions/diseases or aging. In another aspect, cognition is “normal” but patients are deemed “at risk” based on their proinflammatory endophenotype. In another aspect, the level of expression of the various proteins is measured by at least one of fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In another aspect, the method further comprises the step of generating a dataset that comprises expression data from the two or more biomarkers prior to the step of generating a high and a low metabolic endophenotype by determining the level of expression of two or more markers.

In another embodiment, the present invention includes a method of determining the effectiveness of a candidate drug that impacts the metabolism to evaluate the candidate drug believed to be useful in treating and/or preventing cognitive loss, the method comprising: (a) measuring one or more biomarkers in a sample of serum or plasma obtained from a subject suspected of having cognitive loss selected from A2M, fatty acid binding protein (FABP), pancreatic polypeptide (PPP), glucagon like peptide 1 (GLP-1), peptide YY (PYY), insulin, HbA1c, glucose, triglycerides, HDL, LDL, vLDL, DGAT1, PPAR-γ, PPARα, cholesterol, BMI, waist circumference; (b) administering the candidate drug to a first subset of the patients, and a placebo to a second subset of the patients; (c) generating the metabolic groups using one or a combination of the one or more biomarkers; (d) determining the tertile of the level of expression of the one or more biomarkers; and depending on the level of expression dividing the level of expression of the one or more markers as being either high metabolic dysfunction or low metabolic dysfunction; (e) determining if baseline metabolic group predicted treatment response such that the high metabolic group responded differentially than the low metabolic dysfunction group; (f) repeating step (a) after the administration of the candidate drug or the placebo; (g) determining if the candidate drug modifies the metabolic profile over the course of the trial; and (h) determining if change in the metabolic profile over the course of the trial predicted a positive response, a negative response, or a no treatment response, and if a statistically significant treatment response with the candidate drug is obtained. In another aspect, the method further comprises the steps of obtaining one or more additional blood samples from the patient after a pre-determined amount of time and comparing the levels of the biomarkers from the one or more additional samples to determine progression of cognitive loss. In another aspect, the method further comprises the steps of treating the patient for a pre-determined period of time, obtaining one or more additional blood samples from the patient after the pre-determined amount of time and comparing the levels of the biomarkers from the one or more additional samples to determine progression of cognitive loss.

In another embodiment, the present invention includes a method of determining the effectiveness of a candidate drug that impacts metabolism to evaluate the candidate drug believed to be useful in treating or preventing cognitive loss, the method comprising: (a) measuring the serum or plasma based levels of two or more markers selected from fatty acid binding protein, CD40, glucagon like protein-1 (GLP-1), IgM, β-2 microglobulin, IGF-binding protein 2, IL-8, peptide YY, macrophage derived chemokine (MDC), macrophage inflammatory protein-1 (MIP-1 alpha), pancreatic polypeptide, vLDL, DGAT1, PPAR-γ, PPARα; (b) administering a candidate drug to a first subset of the patients, and a placebo to a second subset of the patients; (c) generating the metabolic groups using a combination of the two or more biomarkers for the first and second subset of patients; (d) determining the tertile of the level of expression of the two or more biomarkers in the first and second subset of patients; (e) dividing the level of expression of the two or more biomarkers as being either high metabolic or low metabolic depending on the level of expression of the two or more biomarkers; (f) determining if baseline metabolic group predicted treatment response such that the high metabolic group responded differentially than the low metabolic group, (g) repeating step (a) after the administration of the candidate drug or the placebo; and (h) determining if the candidate drug modifies the metabolism profile over the course of the trial. In one aspect, the method further comprises the step of determining if change in the metabolic profile based on the two or more biomarkers over the course of the trial predicted both positive and negative treatment response as well as no treatment response and if a statistically significant treatment response for the candidate drug was achieved as a primary or secondary outcome of the clinical trial. In one aspect, the method further comprises the steps of obtaining one or more additional blood samples from the patient after a pre-determined amount of time and comparing the levels of FABP and PPP from the one or more additional samples to determine progression of cognitive loss. In another aspect, the method further comprises the steps of: treating the patient for a pre-determined period of time, obtaining one or more additional blood samples from the patient after the pre-determined amount of time and comparing the levels of the biomarkers from the one or more additional samples to determine progression of cognitive loss.

In another embodiment, the present invention includes a method for selecting a therapy for improved cognition or prevention of cognitive loss using one or more anti-diabetic therapies for subjects of Mexican-American ethnogenicity comprising: obtaining a sample from a Mexican-American subject; generating a high and a low metabolic endophenotype by determining the level of expression of two or more markers selected from fatty acid binding protein (FABP), CD40, glucagon like protein-1 (GLP-1), IgM, β-2 microglobulin, IGF-binding protein 2, IL-8, peptide YY, macrophage derived chemokine (MDC), macrophage inflammatory protein-1 (MIP-1 alpha), pancreatic polypeptide, glycated hemoglobin A1c (HbA1c), glucose, triglycerides, high density lipoprotein (HDL), low density lipoproteins (LDL and vLDL), DGAT1, PPAR-γ, PPARα, cholesterol, body mass index (BMI), or waist circumference; comparing the level of the one or more biomarkers within a sample of patients suffering from cognitive loss; dividing the level of expression of the one or more markers as being either high metabolic or low metabolic; and selecting a course of treatment for the subject based on whether the subject is selected as being high metabolic or low metabolic, wherein a high metabolic subject benefits from a treatment with an anti-diabetic drug.

In another embodiment, the present invention includes a method of conducting a clinical trial of a drug that impacts metabolism of subjects of Mexican-American ethnogenetics to evaluate the candidate drug believed to be useful in treating and/or preventing cognitive loss, the method comprising: (a) measuring the serum or plasma based levels of two or more markers selected from two or more markers selected from fatty acid binding protein (FABP), CD40, glucagon like protein-1 (GLP-1), IgM, β-2 microglobulin, IGF-binding protein 2, IL-8, peptide YY, macrophage derived chemokine (MDC), macrophage inflammatory protein-1 (MIP-1 alpha), pancreatic polypeptide, and one or more physiological markers selected from glycated hemoglobin A1c (HbA1c), glucose, triglycerides, high density lipoprotein (HDL), low density lipoproteins (LDL, vLDL), DGAT1, PPAR-γ, PPARα, cholesterol, body mass index (BMI), or waist circumference; (b) administering a candidate drug to a first subset of the patients, and a placebo to a second subset of the patients; (c) generating the metabolic groups using a combination of the two or more biomarkers for the first and second subset of patients; (d) determining the tertile of the level of expression of the two or more biomarkers in the first and second subset of patients; (e) dividing the level of expression of the two or more biomarkers as being either high metabolic or low metabolic depending on the level of expression of the two or more biomarkers; and (f) determining if baseline metabolic group predicted treatment response such that the high metabolic group responded differentially than the low metabolic group, (g) repeating step (a) after the administration of the candidate drug or the placebo; and (h) determining if the candidate drug modifies the metabolism profile over the course of the trial.

In another embodiment, the present invention includes a method for selecting a therapy for improved cognition or prevention of cognitive loss using one or more neurotrophic factor therapies (agonists) comprising: obtaining a sample from a subject; measuring one or more biomarkers in the sample selected from BDNF, NGF, TN-3, CNTF, GDNF, LIF, and GGF; comparing the level of the one or more biomarkers within a sample of patients suffering from cognitive loss; dividing the level of expression of the one or more biomarkers as being either high neurotrophic or low neurotrophic; and selecting a course of treatment for the subject based on whether the subject is selected as being high neurotrophic endophenotype or low neurotrophic endophenotype. In one aspect, the method further comprises the steps of: generating a high and a low neurotrophic endophenotype by determining the level of expression of 2, 3, 4, 5, 6, or 7 biomarkers selected from brain derived neurotrophic factor (BDNF), nerve growth factor (NGF), tenascin 3 (TN-3), ciliary neurotrophic factor (CNTF), glial cell derived neurotrophic factor (GDNF), leukemia inhibitory factor (LIF), and neuregulin-1 (GGF); and determining the high and low neurotrophic endophenotypes by determining the level of expression of 2, 3, 4, 5, 6, or 7 more biomarkers. In one aspect, the neurotrophic profile is generated using learning machines (random forest, support vector machines), clustering algorithms (factor analysis, principal component analysis), summation of values, or other methods to generate a neurotrophic score across multiple measures. In another aspect, the high end of the score across multiple markers is reflective of the high neurotrophic endophenotype and the low end as the low neurotrophic endophenotype with all others falling in a middle endophenotype. In another aspect, if the subject is scored in the low neurotrophic group a neurotrophic-factor treatment is indicated to maintain cognitive ability, and if the subject is scored in a high neurotrophic group then a neurotrophic-factor treatment may be indicated to boost cognitive ability, but may be contraindicated in some patients. In another aspect, at least one of the biomarker measurements is obtained by a method selected from the group consisting of immunoassay and enzymatic activity assay. In another aspect, the sample is serum or plasma. In another aspect, the cognitive dysfunction is a disease or condition selected from Alzheimer's Disease, Parkinson's Disease, Down's syndrome, Frontotemporal dementia, Dementia with Lewy Bodies, Multiple sclerosis, traumatic brain injury, depression, schizophrenia, bipolar disease (and other mental illness), diabetes, hypertension, stroke, heart attack, dyslipidemia, other conditions/diseases or aging. In another aspect, cognition is “normal” but patients are deemed “at risk” based on their proinflammatory endophenotype. In another aspect, the level of expression of the various proteins is measured by at least one of fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In another aspect, the high and low end of the neurotrophic group is determined by specifically determining the level of expression of BDNF, NGF, and TN-3. In another aspect, the neurotrophic endophenotype may be treated with one of more of the following non-limiting examples of therapeutic agents: Neurotrophic factor agonist, exercise therapy, brain derived neurotrophic factor (BDNF) and BDNF agonists, selective serotonin reuptake inhibitors, selective serotonin 2C (5-HT2C) antagonists, serotonin-norepinephrine reuptake inhibitors, tricyclic, combined exercise and medications, glial-cell derived neurotrophic factor (GDNF) and GDNF agonists.

In another embodiment, the present invention includes a method for selecting patient therapy for improved cognition or prevention of cognitive loss comprising: obtaining a sample from a subject; measuring the level of expression of brain derived neurotrophic factor (BDNF), nerve growth factor (NGF), and tenascin 3 (TN-3); determining the tertile of the level of expression of the these three biomarkers; and depending on the level of expression dividing the level of expression of the two or more markers as being either high neurotrophic endophenotype or low neurotrophic endophenotype; and selecting a course of treatment for the subject based on whether the subject is selected as being high neurotrophic endophenotype or low neurotrophic endophenotype, wherein the tertile is determined by: scoring the tertile scores for both markers to generate a score with a range from two to six, assigning a lower score (i.e., 3) to the low end of a neurotrophic, assigning a highest score (i.e., 9) score was assigned to a high end of the neurotrophic, with all other scores falling in a middle score. In one aspect, if the subject is scored in the tertile that is scored as a high neurotrophic a neurotrophic-factor treatment may be indicated to preserve remaining cognitive ability, and if the subject is scored in a low neurotrophic endophenotype then a neurotrophic-factor treatment (agonist) is indicated to improve and maintain cognition. In another aspect, the sample is serum or plasma. In another aspect, the cognitive dysfunction is a disease or condition selected from Alzheimer's Disease, Parkinson's Disease, Down's syndrome, Frontotemporal dementia, Dementia with Lewy Bodies, Multiple sclerosis, traumatic brain injury, depression, schizophrenia, bipolar disease (and other mental illness), diabetes, hypertension, stroke, heart attack, dyslipidemia, other conditions/diseases or aging. In another aspect, cognition is “normal” but patients are deemed “at risk” based on their pro-inflammatory endophenotype. In another aspect, the level of expression of the various proteins is measured by at least one of fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling.

In another embodiment, the present invention includes a method of determining the effectiveness of a candidate drug that impacts the neurotrophic system to evaluate the candidate drug believed to be useful in treating and/or preventing cognitive loss, the method comprising: (a) measuring one or more biomarkers in a sample of serum or plasma obtained from a subject suspected of having cognitive loss selected from BDNF, NGF, TN-3, CNTF, GDNF, LIF, and GGF; (b) administering the candidate drug to a first subset of the patients, and a placebo to a second subset of the patients; (c) generating the neurotrophic endophenotype groups using one or a combination of the one or more biomarkers; (d) determining the tertile of the level of expression of the one or more biomarkers; and depending on the level of expression dividing the level of expression of the one or more markers as being either high neurotrophic or low neurotrophic; (e) determining if baseline neurotrophic endophenotype group predicted treatment response such that the high and low neurotrophic endophenotype groups responded differentially than the middle neurotrophic endophenotype group; (f) repeating step (a) after the administration of the candidate drug or the placebo; (g) determining if the candidate drug modifies the neurotrophic profile over the course of the trial; and (h) determining if change in the pro neurotrophic profile over the course of the trial predicted a positive response, a negative response, or a no treatment response, and if a statistically significant treatment response for cognitive loss with the candidate drug is obtained. In one aspect, the method further comprises the steps of obtaining one or more additional blood samples from the patient after a pre-determined amount of time and comparing the levels of the biomarkers from the one or more additional samples to determine progression of cognitive loss. In one aspect, the method further comprises the steps of treating the patient for a pre-determined period of time, obtaining one or more additional blood samples from the patient after the pre-determined amount of time and comparing the levels of the biomarkers from the one or more additional samples to determine progression of cognitive loss.

In another embodiment, the present invention includes a method of determining the effectiveness of a candidate drug that impacts the neurotrophic system to evaluate the candidate drug believed to be useful in treating and/or preventing cognitive loss, the method comprising: (a) measuring the serum or plasma based levels of BDNF, NGF, and TN-3; (b) administering the candidate drug to a first subset of the patients, and a placebo to a second subset of the patients; (c) generating the neurotrophic groups using a combination of BDNF, NGF, and TN-3 for the first and second subset of patients; (d) determining the tertile of the level of expression of BDNF, NGF, and TN-3 in the first and second subset of patients; (e) dividing the level of expression of BDNF, NGF, and TN-3 as being either high neurotrophic or low neurotrophic depending on the level of expression of BDNF, NGF, and TN-3; (f) determining if baseline neurotrophic group predicted treatment response such that the high neurotrophic group responded differentially than the low neurotrophic group, (g) repeating step (a) after the administration of the candidate drug or the placebo; and (h) determining if the candidate drug modifies the neurotrophic profile over the course of the trial. In one aspect, the method further comprises the step of determining if change in the pro neurotrophic profile based on the one or more neurotrophic biomarkers over the course of the trial predicted both positive and negative treatment response as well as no treatment response and if a statistically significant treatment response for the candidate drug was achieved as a primary or secondary outcome of the clinical trial. In another aspect, the method further comprises the steps of obtaining one or more additional blood samples from the patient after a pre-determined amount of time and comparing the levels of the one or more neurotrophic biomarkers from the one or more additional samples to determine progression of cognitive loss. In one aspect, the method further comprises the steps of: treating the patient for a pre-determined period of time, obtaining one or more additional blood samples from the patient after the pre-determined amount of time and comparing the levels of the biomarkers from the one or more additional samples to determine progression of cognitive loss.

In another embodiment, the present invention includes a method for selecting a therapy for improved cognition or prevention of cognitive loss using one or more endophenotypes comprising: obtaining a sample from a subject; measuring biomarkers that differentiate between an inflammatory, a metabolic, and a neurotrophic endophenotype; and selecting a course of treatment for the subject based on whether the subject is scored as having a high or a low endophenotype for one or more of the inflammatory, a metabolic, and a neurotrophic. In another aspect, the endophenotype profile is generated using learning machines (random forest, support vector machines), clustering algorithms (factor analysis, principal component analysis), summation of values, or other methods to generate an endophenotype score across multiple measures. In another aspect, if the subject is scored elevated for inflammatory endophenotype an anti-inflammatory treatment is indicated. In another aspect, if the subject is scored elevated for metabolic endophenotype and anti-metabolic treatment is indicated. In another aspect, if the subject is scored elevated for neurotrophic endophenotype a neurotrophic treatment is indicated. In another aspect, the cognitive dysfunction is a disease or condition selected from Alzheimer's Disease, Parkinson's Disease, Down's syndrome, Frontotemporal dementia, Dementia with Lewy Bodies, Multiple sclerosis, traumatic brain injury, depression, schizophrenia, bipolar disease (and other mental illness), diabetes, hypertension, stroke, heart attack, dyslipidemia, other conditions/diseases or the aging process itself. In another aspect, cognition is “normal” but patients are deemed “at risk” based on their pro-inflammatory endophenotype.

In another embodiment, the present invention includes an apparatus for selecting a therapy for improved cognition or preventing cognitive loss using one or more endophenotypes comprising: a biomarker array that detects biomarkers and computerized questions/cognitive assessments from a sample for two or more endophenotypes selected from an inflammatory, a metabolic, and a neurotrophic endophenotype; a processor/algorithm that obtains a biomarker and questionnaire/cognitive test results expression output from the biomarker array, wherein an endophenotype profile is generated using learning machines (random forest, support vector machines), clustering algorithms (factor analysis, principal component analysis), summation of values, or other methods to generate an endophenotype score across multiple measures; and an output that indicates a course of treatment for the subject based on whether the subject is scored as having a high or a low endophenotype for two or more of the inflammatory, metabolic, or neurotrophic endophenotypes. In another aspect, if the subject is scored elevated for inflammatory endophenotype an anti-inflammatory treatment is indicated. In another aspect, if the subject is scored elevated for metabolic endophenotype and anti-metabolic treatment is indicated. In another aspect, if the subject is scored altered (elevated and/or suppressed) for neurotrophic endophenotype a neurotrophic treatment is indicated. In another aspect, the cognitive dysfunction is a disease or condition selected from Alzheimer's Disease, Parkinson's Disease, Down's syndrome, Frontotemporal dementia, Dementia with Lewy Bodies, Multiple sclerosis, traumatic brain injury, depression, schizophrenia, bipolar disease (and other mental illness), diabetes, hypertension, stroke, heart attack, dyslipidemia, other conditions/diseases or the aging process itself. In another aspect, cognition is “normal” but patients are deemed “at risk” based on their pro-inflammatory endophenotype.

In another embodiment, the present invention includes a method for selecting patients to determine the effectiveness of a candidate drug comprising: generating a prediction model dataset by: pre-selecting a level of treatment response selected from positive, negative and no response for a patient dataset within an endophenotype; obtaining the patient dataset based on the endophenotype selected; and separating the patient dataset into a responder patient dataset, non-responder patient dataset and adverse responder patient dataset; applying the prediction model blindly to a second clinical trial dataset to predict outcomes; and determining the efficacy of the prediction model in predicting treatment responders, non-responders and adverse responders in a third trial, wherein the efficacy for the third trial is increased by only evaluating a patient outcome from the responder patient dataset. In one aspect, the one or more outcome variable datasets are preselected based on the endophenotypes. In another aspect, the method further comprises selecting one or more additional endophenotypes for evaluation. In another aspect, the method further comprises the step of selecting one or more patients for targeted therapy, designing a new clinical trial that specifically targets only those patients most likely to respond, or both.

Method of Selecting a Subject for Treatment with Naproxen or Celecoxib

In yet another embodiment, the present disclosure provides a method of selecting a subject for treatment with an NSAID, the method comprising: obtaining a plasma or serum sample from the subject; measuring in the sample the expression level of one or more proinflammatory biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY); determining that the subject has a proinflammatory endophenotype profile based on the expression level of the proinflammatory biomarkers; and selecting the subject for treatment with an NSAID selected from naproxen and celecoxib.

In one aspect, the sample is a blood sample.

In one aspect, the subject is an elderly subject. In one aspect, the subject is a human subject. In one aspect, the subject is 50 years of age or older. In one aspect, the subject has an elevated risk of age-related memory loss, senility, dementia, Alzheimer's Disease, or a combination thereof due to a family history of one or more of these diseases or disorders. In one aspect, the subject has a family history of one of these diseases or disorders and is selected for treatment to prevent the subject from developing the disease or disorder. In some aspects, the subject has a family history of Alzheimer's disease and is selected for treatment to prevent the subject from developing Alzheimer's disease. In some aspects, the subject is suspected of having age-related memory loss, senility, dementia, or Alzheimer's Disease due to the subject's advanced age and complaints of cognitive decline or dysfunction from the subject or the subject's caretaker. In another aspect, the subject is suspected of having age-related memory loss, senility, dementia, or Alzheimer's Disease due to the results of one or more neurocognitive evaluations.

In one aspect, the expression level of one or more biomarkers selected from IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and I309 in the plasma or serum sample is compared to the average level of expression of the one or more corresponding biomarkers in a statistical sample representative of the subject. In some aspects, the statistical sample comprises elderly individuals with a family history of memory loss, senility, dementia, or Alzheimer's disease. In other aspects, the statistical sample comprises elderly individuals who do not have a family history of memory loss, senility, dementia, or Alzheimer's disease. In some aspects, the statistical sample comprises the measurements of the expression level for one or more biomarkers selected from IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and I309 from each elderly individual in the statistical sample.

In one aspect, the plasma or serum sample obtained from the subject is found to have a statistically different expression level of the one or more biomarkers when compared to the expression level of the corresponding biomarkers from elderly individuals with a family history of memory loss, senility, dementia, or Alzheimer's disease in the statistical sample. In some aspects, this indicates that the subject does not have a proinflammatory endophenotype profile and the subject is not selected for NSAID treatment. In another aspect, the plasma or serum sample obtained from the subject is found to have a statistically similar expression level of the one or more biomarkers when compared to the expression level of the corresponding biomarkers from elderly individuals who do not have a family history of memory loss, senility, dementia, or Alzheimer's disease in the statistical sample. In some aspects, this indicates that the subject does not have a proinflammatory endophenotype profile and the subject is not selected for NSAID treatment.

In another aspect, the plasma or serum sample obtained from the subject is found to have a statistically similar expression level of the one or more biomarkers when compared to the expression level of the corresponding biomarkers from elderly individuals with a family history of memory loss, senility, dementia, or Alzheimer's disease in the statistical sample. In some aspects, this indicates that the subject has a proinflammatory endophenotype profile and the subject is selected for NSAID treatment. In another aspect, the plasma or serum sample obtained from the subject is found to have a statistically different expression level of the one or more biomarkers when compared to the expression level of the corresponding biomarkers from elderly individuals who do not have a family history of memory loss, senility, dementia, or Alzheimer's disease in the statistical sample. In some aspects, this indicates that the subject has a proinflammatory endophenotype profile and the subject is selected for NSAID treatment.

In some aspects, the expression level of the one or more biomarkers in the plasma or serum sample obtained from the subject is elevated when compared to the expression level of the corresponding biomarkers the statistical sample. In another aspect, the expression level of the one or more biomarkers in the plasma or serum sample obtained from the subject is reduced when compared to the expression level of the corresponding biomarkers the statistical sample. In yet another aspect, the expression level of one or more of the biomarkers in the plasma or serum sample obtained from the subject is elevated and the expression level of one or more of the biomarkers in the plasma or serum sample obtained from the subject is reduced compared to the expression level of the corresponding biomarkers the statistical sample.

In some aspects, the step of measuring the expression level of the one or more biomarkers in the plasma or serum sample comprises measuring the expression level of each of IL-10 and eotaxin 3; IL-10, eotaxin 3, and A2M; IL-10, eotaxin 3, A2M, and SAA; IL-10, eotaxin 3, A2M, SAA, and tenacin C; IL-10, eotaxin 3, A2M, SAA, tenacin C, and FABP; IL-10, eotaxin 3, A2M, SAA, and tenacin C; IL-10, eotaxin 3, A2M, SAA, tenacin C, FABP, and IL-5; IL-10, eotaxin 3, A2M, SAA, and tenacin C; IL-10, eotaxin 3, A2M, SAA, tenacin C, FABP, IL-5, and TPO; IL-10, eotaxin 3, A2M, SAA, and tenacin C; IL-10, eotaxin 3, A2M, SAA, tenacin C, FABP, IL-5, TPO, and IL-6; IL-10, eotaxin 3, A2M, SAA, tenacin C, FABP, IL-5, TPO, IL-6, and Factor VII; IL-10, eotaxin 3, A2M, SAA, tenacin C, FABP, IL-5, TPO, IL-6, Factor VII, and IL-18; IL-10, eotaxin 3, A2M, SAA, tenacin C, FABP, IL-5, TPO, IL-6, Factor VII, IL-18, and TARC; IL-10, eotaxin 3, A2M, SAA, tenacin C, FABP, IL-5, TPO, IL-6, Factor VII, IL-18, TARC, and TNFα; IL-10, eotaxin 3, A2M, SAA, tenacin C, FABP, IL-5, TPO, IL-6, Factor VII, IL-18, TARC, TNFα, and sVCAM-1; IL-10, eotaxin 3, A2M, SAA, tenacin C, FABP, IL-5, TPO, IL-6, Factor VII, IL-18, TARC, TNFα, sVCAM-1, and CRP; IL-10, eotaxin 3, A2M, SAA, tenacin C, FABP, IL-5, TPO, IL-6, Factor VII, IL-18, TARC, TNFα, sVCAM-1, CRP, and IL-7; IL-10, eotaxin 3, A2M, SAA, tenacin C, FABP, IL-5, TPO, IL-6, Factor VII, IL-18, TARC, TNFα, sVCAM-1, CRP, IL-7, and sICAM-1; IL-10, eotaxin 3, A2M, SAA, tenacin C, FABP, IL-5, TPO, IL-6, Factor VII, IL-18, TARC, TNFα, sVCAM-1, CRP, IL-7, and sICAM-1; IL-10, eotaxin 3, A2M, SAA, tenacin C, FABP, IL-5, TPO, IL-6, Factor VII, IL-18, TARC, TNFα, sVCAM-1, CRP, IL-7, sICAM-1, and B2M; IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, and PPY; or IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and I309. In some aspects, the expression level of each of the biomarkers measured above is compared to the expression level of each of the corresponding biomarkers in the statistical sample to determine if the subject has a proinflammatory endophenotype and to select the subject for treatment with celecoxib.

In some aspects, the subject is selected for treatment with celecoxib when the plasma or serum sample obtained from the subject is found to have a statistically different expression level of one or more biomarkers selected from IL-10, eotaxin 3, A2M, SAA, Tenacin C, FABP, IL-5, and TPO when compared to the expression level of the corresponding biomarkers from elderly individuals who do not have a family history of memory loss, senility, dementia, or Alzheimer's disease in the statistical sample. In some aspects, the subject is selected for treatment with celecoxib when the plasma or serum sample obtained from the subject is found to have a statistically similar expression level of one or more biomarkers selected from IL-10, eotaxin 3, A2M, SAA, Tenacin C, FABP, IL-5, and TPO when compared to the expression level of the corresponding biomarkers from elderly individuals who have a family history of memory loss, senility, dementia, or Alzheimer's disease in the statistical sample. In one aspect, the subject is selected for treatment with celecoxib when the sample obtained from the subject is found to have a statistically different expression level of each of IL-10, eotaxin 3, A2M, and SAA, and optionally Tenacin C when compared to the expression level of the corresponding biomarkers from elderly individuals who do not have a family history of memory loss, senility, dementia, or Alzheimer's disease in the statistical sample. In another aspect, the subject is selected for treatment with celecoxib when the sample obtained from the subject is found to have a statistically similar expression level of each of IL-10, eotaxin 3, A2M, and SAA, and optionally Tenacin C when compared to the expression level of the corresponding biomarkers from elderly individuals who have a family history of memory loss, senility, dementia, or Alzheimer's disease in the statistical sample.

In other aspects, the step of measuring the expression level of the one or more biomarkers in the plasma or serum sample comprises measuring the expression level of each of TPO and IL-5; TPO, IL-5, and IL-7; TPO, IL-5, IL-7, and I309; TPO, IL-5, IL-7, I309, and IL-6; TPO, IL-5, IL-7, I309, IL-6, and SAA; TPO, IL-5, IL-7, I309, IL-6, SAA, and PPY; TPO, IL-5, IL-7, I309, IL-6, SAA, PPY, and A2M; TPO, IL-5, IL-7, I309, IL-6, SAA, PPY, A2M, and TARC; TPO, IL-5, IL-7, I309, IL-6, SAA, PPY, A2M, TARC, and CRP; TPO, IL-5, IL-7, I309, IL-6, SAA, PPY, A2M, TARC, CRP, and sVCAM-1; TPO, IL-5, IL-7, I309, IL-6, SAA, PPY, A2M, TARC, CRP, sVCAM-1, and sICAM-1; TPO, IL-5, IL-7, I309, IL-6, SAA, PPY, A2M, TARC, CRP, sVCAM-1, sICAM-1, and B2M; TPO, IL-5, IL-7, I309, IL-6, SAA, PPY, A2M, TARC, CRP, sVCAM-1, sICAM-1, B2M, and Factor VII; TPO, IL-5, IL-7, I309, IL-6, SAA, PPY, A2M, TARC, CRP, sVCAM-1, sICAM-1, B2M, Factor VII, and IL-10; TPO, IL-5, IL-7, I309, IL-6, SAA, PPY, A2M, TARC, CRP, sVCAM-1, sICAM-1, B2M, Factor VII, IL-10, and tenacin C; TPO, IL-5, IL-7, I309, IL-6, SAA, PPY, A2M, TARC, CRP, sVCAM-1, sICAM-1, B2M, Factor VII, IL-10, tenacin C, and eotaxin 3; TPO, IL-5, IL-7, I309, IL-6, SAA, PPY, A2M, TARC, CRP, sVCAM-1, sICAM-1, B2M, Factor VII, IL-10, tenacin C, eotaxin 3, and IL-18; TPO, IL-5, IL-7, I309, IL-6, SAA, PPY, A2M, TARC, CRP, sVCAM-1, sICAM-1, B2M, Factor VII, IL-10, tenacin C, eotaxin 3, IL-18, and FABP; or TPO, IL-5, IL-7, I309, IL-6, SAA, PPY, A2M, TARC, CRP, sVCAM-1, sICAM-1, B2M, Factor VII, IL-10, tenacin C, eotaxin 3, IL-18, FABP, and TNFα. In some aspects, the expression level of each of the biomarkers measured above is compared to the expression level of each of the corresponding biomarkers in the statistical sample to determine if the subject has a proinflammatory endophenotype and to select the subject for treatment with naproxen.

In one aspect, the subject is selected for treatment with naproxen when the plasma or serum sample obtained from the subject is found to have a statistically different expression level of one or more biomarkers selected from TPO, IL-5, IL-7, I309, IL-6, SAA, PPY, and A2M when compared to the expression level of the corresponding biomarkers from elderly individuals who do not have a family history of memory loss, senility, dementia, or Alzheimer's disease in the statistical sample. In another aspect, the subject is selected for treatment with naproxen when the plasma or serum sample obtained from the subject is found to have a statistically similar expression level of one or more biomarkers selected from TPO, IL-5, IL-7, I309, IL-6, SAA, PPY, and A2M when compared to the expression level of the corresponding biomarkers from elderly individuals who have a family history of memory loss, senility, dementia, or Alzheimer's disease in the statistical sample. In one aspect, the subject is selected for treatment with naproxen when the plasma or serum sample obtained from the subject is found to have a statistically different expression level of each of TPO, IL-5, IL-7, and I309, and optionally IL-6 when compared to the expression level of the corresponding biomarkers from elderly individuals who do not have a family history of memory loss, senility, dementia, or Alzheimer's disease in the statistical sample. In another aspect, the subject is selected for treatment with naproxen when the plasma or serum sample obtained from the subject is found to have a statistically similar expression level of each of TPO, IL-5, IL-7, and I309, and optionally IL-6 when compared to the expression level of the corresponding biomarkers from elderly individuals who have a family history of memory loss, senility, dementia, or Alzheimer's disease in the statistical sample.

In one aspect, the biomarker measurements are obtained by a method selected from the group consisting of an immunoassay, an enzymatic activity assay, fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, and radioactive labeling. In some aspects, the biomarker measurements are obtained by electrochemiluminescence detection.

Method of Treating a Subject with Naproxen or Celecoxib

In yet another embodiment, the present disclosure provides a method of treating a subject to improve cognition or to prevent cognitive decline or dysfunction in the subject, the method comprising: obtaining a plasma or serum sample from the subject; measuring in the sample the expression level of one or more proinflammatory biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY), and determining that the subject has a proinflammatory endophenotype profile based on the expression level of the proinflammatory biomarkers; and administering an NSAID selected from naproxen and celecoxib to the subject.

In one aspect, the sample is a blood sample.

In one aspect, the subject is an elderly subject. In one aspect, the subject is a human subject. In one aspect, the subject is 50 years of age or older. In some aspects, the subject has a family history of age-related memory loss, senility, dementia, Alzheimer's Disease, or a combination thereof. In some aspects, the subject is treated to improve cognition or cognitive decline or dysfunction related to old age, including but not limited to, memory loss, senility, dementia, or a combination thereof. In some aspects, the subject is treated to improve cognition or cognitive decline or dysfunction related to Alzheimer's disease. In another aspect, the subject is treated to delay or prevent the onset of Alzheimer's disease.

In one aspect, the expression level of one or more biomarkers selected from IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and I309 in the plasma or serum sample is compared to the average level of expression of the one or more corresponding biomarkers in a statistical sample representative of the subject. In some aspects, the statistical sample comprises elderly individuals with a family history of memory loss, senility, dementia, or Alzheimer's disease. In other aspects, the statistical sample comprises elderly individuals who do not have a family history of memory loss, senility, dementia, or Alzheimer's disease. In some aspects, the statistical sample comprises the measurements of the expression level for one or more biomarkers selected from IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and I309 from each elderly individual in the statistical sample.

In one aspect, the plasma or serum sample obtained from the subject is found to have a statistically different expression level of the one or more biomarkers when compared to the expression level of the corresponding biomarkers from elderly individuals with a family history of memory loss, senility, dementia, or Alzheimer's disease in the statistical sample. In some aspects, this indicates that the subject does not have a proinflammatory endophenotype profile and the subject is not administered an NSAID. In another aspect, the plasma or serum sample obtained from the subject is found to have a statistically similar expression level of the one or more biomarkers when compared to the expression level of the corresponding biomarkers from elderly individuals who do not have a family history of memory loss, senility, dementia, or Alzheimer's disease in the statistical sample. In some aspects, this indicates that the subject does not have a proinflammatory endophenotype profile and the subject is not administered an NSAID.

In another aspect, the plasma or serum sample obtained from the subject is found to have a statistically similar expression level of the one or more biomarkers when compared to the expression level of the corresponding biomarkers from elderly individuals with a family history of memory loss, senility, dementia, or Alzheimer's disease in the statistical sample. In some aspects, this indicates that the subject has a proinflammatory endophenotype profile and the subject is administered an NSAID. In another aspect, the plasma or serum sample obtained from the subject is found to have a statistically different expression level of the one or more biomarkers when compared to the expression level of the corresponding biomarkers from elderly individuals who do not have a family history of memory loss, senility, dementia, or Alzheimer's disease in the statistical sample. In some aspects, this indicates that the subject has a proinflammatory endophenotype profile and the subject is administered an NSAID.

In some aspects, the expression level of the one or more biomarkers in the plasma or serum sample obtained from the subject is elevated when compared to the expression level of the corresponding biomarkers the statistical sample. In another aspect, the expression level of the one or more biomarkers in the plasma or serum sample obtained from the subject is reduced when compared to the expression level of the corresponding biomarkers the statistical sample. In yet another aspect, the expression level of one or more of the biomarkers in the plasma or serum sample obtained from the subject is elevated and the expression level of one or more of the biomarkers in the plasma or serum sample obtained from the subject is reduced compared to the expression level of the corresponding biomarkers the statistical sample.

In some aspects, the step of measuring the expression level of the one or more biomarkers in the plasma or serum sample comprises measuring the expression level of each of IL-10 and eotaxin 3; IL-10, eotaxin 3, and A2M; IL-10, eotaxin 3, A2M, and SAA; IL-10, eotaxin 3, A2M, SAA, and tenacin C; IL-10, eotaxin 3, A2M, SAA, tenacin C, and FABP; IL-10, eotaxin 3, A2M, SAA, and tenacin C; IL-10, eotaxin 3, A2M, SAA, tenacin C, FABP, and IL-5; IL-10, eotaxin 3, A2M, SAA, and tenacin C; IL-10, eotaxin 3, A2M, SAA, tenacin C, FABP, IL-5, and TPO; IL-10, eotaxin 3, A2M, SAA, and tenacin C; IL-10, eotaxin 3, A2M, SAA, tenacin C, FABP, IL-5, TPO, and IL-6; IL-10, eotaxin 3, A2M, SAA, tenacin C, FABP, IL-5, TPO, IL-6, and Factor VII; IL-10, eotaxin 3, A2M, SAA, tenacin C, FABP, IL-5, TPO, IL-6, Factor VII, and IL-18; IL-10, eotaxin 3, A2M, SAA, tenacin C, FABP, IL-5, TPO, IL-6, Factor VII, IL-18, and TARC; IL-10, eotaxin 3, A2M, SAA, tenacin C, FABP, IL-5, TPO, IL-6, Factor VII, IL-18, TARC, and TNFα; IL-10, eotaxin 3, A2M, SAA, tenacin C, FABP, IL-5, TPO, IL-6, Factor VII, IL-18, TARC, TNFα, and sVCAM-1; IL-10, eotaxin 3, A2M, SAA, tenacin C, FABP, IL-5, TPO, IL-6, Factor VII, IL-18, TARC, TNFα, sVCAM-1, and CRP; IL-10, eotaxin 3, A2M, SAA, tenacin C, FABP, IL-5, TPO, IL-6, Factor VII, IL-18, TARC, TNFα, sVCAM-1, CRP, and IL-7; IL-10, eotaxin 3, A2M, SAA, tenacin C, FABP, IL-5, TPO, IL-6, Factor VII, IL-18, TARC, TNFα, sVCAM-1, CRP, IL-7, and sICAM-1; IL-10, eotaxin 3, A2M, SAA, tenacin C, FABP, IL-5, TPO, IL-6, Factor VII, IL-18, TARC, TNFα, sVCAM-1, CRP, IL-7, and sICAM-1; IL-10, eotaxin 3, A2M, SAA, tenacin C, FABP, IL-5, TPO, IL-6, Factor VII, IL-18, TARC, TNFα, sVCAM-1, CRP, IL-7, sICAM-1, and B2M; IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, and PPY; or IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and I309. In some aspects, the expression level of each of the biomarkers measured above is compared to the expression level of each of the corresponding biomarkers in the statistical sample to determine if the subject has a proinflammatory endophenotype and then administer celecoxib to the subject.

In some aspects, celecoxib is administered to the subject when the plasma or serum sample obtained from the subject is found to have a statistically different expression level of one or more biomarkers selected from IL-10, eotaxin 3, A2M, SAA, Tenacin C, FABP, IL-5, and TPO when compared to the expression level of the corresponding biomarkers from elderly individuals who do not have a family history of memory loss, senility, dementia, or Alzheimer's disease in the statistical sample. In some aspects, celecoxib is administered to the subject when the plasma or serum sample obtained from the subject is found to have a statistically similar expression level of one or more biomarkers selected from IL-10, eotaxin 3, A2M, SAA, Tenacin C, FABP, IL-5, and TPO when compared to the expression level of the corresponding biomarkers from elderly individuals who have a family history of memory loss, senility, dementia, or Alzheimer's disease in the statistical sample. In one aspect, celecoxib is administered to the subject when the sample obtained from the subject is found to have a statistically different expression level of each of IL-10, eotaxin 3, A2M, and SAA, and optionally Tenacin C when compared to the expression level of the corresponding biomarkers from elderly individuals who do not have a family history of memory loss, senility, dementia, or Alzheimer's disease in the statistical sample. In another aspect, celecoxib is administered to the subject when the sample obtained from the subject is found to have a statistically similar expression level of each of IL-10, eotaxin 3, A2M, and SAA, and optionally Tenacin C when compared to the expression level of the corresponding biomarkers from elderly individuals who have a family history of memory loss, senility, dementia, or Alzheimer's disease in the statistical sample.

In other aspects, the step of measuring the expression level of the one or more biomarkers in the plasma or serum sample comprises measuring the expression level of each of TPO and IL-5; TPO, IL-5, and IL-7; TPO, IL-5, IL-7, and I309; TPO, IL-5, IL-7, I309, and IL-6; TPO, IL-5, IL-7, I309, IL-6, and SAA; TPO, IL-5, IL-7, I309, IL-6, SAA, and PPY; TPO, IL-5, IL-7, I309, IL-6, SAA, PPY, and A2M; TPO, IL-5, IL-7, I309, IL-6, SAA, PPY, A2M, and TARC; TPO, IL-5, IL-7, I309, IL-6, SAA, PPY, A2M, TARC, and CRP; TPO, IL-5, IL-7, I309, IL-6, SAA, PPY, A2M, TARC, CRP, and sVCAM-1; TPO, IL-5, IL-7, I309, IL-6, SAA, PPY, A2M, TARC, CRP, sVCAM-1, and sICAM-1; TPO, IL-5, IL-7, I309, IL-6, SAA, PPY, A2M, TARC, CRP, sVCAM-1, sICAM-1, and B2M; TPO, IL-5, IL-7, I309, IL-6, SAA, PPY, A2M, TARC, CRP, sVCAM-1, sICAM-1, B2M, and Factor VII; TPO, IL-5, IL-7, I309, IL-6, SAA, PPY, A2M, TARC, CRP, sVCAM-1, sICAM-1, B2M, Factor VII, and IL-10; TPO, IL-5, IL-7, I309, IL-6, SAA, PPY, A2M, TARC, CRP, sVCAM-1, sICAM-1, B2M, Factor VII, IL-10, and tenacin C; TPO, IL-5, IL-7, I309, IL-6, SAA, PPY, A2M, TARC, CRP, sVCAM-1, sICAM-1, B2M, Factor VII, IL-10, tenacin C, and eotaxin 3; TPO, IL-5, IL-7, I309, IL-6, SAA, PPY, A2M, TARC, CRP, sVCAM-1, sICAM-1, B2M, Factor VII, IL-10, tenacin C, eotaxin 3, and IL-18; TPO, IL-5, IL-7, I309, IL-6, SAA, PPY, A2M, TARC, CRP, sVCAM-1, sICAM-1, B2M, Factor VII, IL-10, tenacin C, eotaxin 3, IL-18, and FABP; or TPO, IL-5, IL-7, I309, IL-6, SAA, PPY, A2M, TARC, CRP, sVCAM-1, sICAM-1, B2M, Factor VII, IL-10, tenacin C, eotaxin 3, IL-18, FABP, and TNFα. In some aspects, the expression level of each of the biomarkers measured above is compared to the expression level of each of the corresponding biomarkers in the statistical sample to determine if the subject has a proinflammatory endophenotype and then administer naproxen to the subject.

In one aspect, naproxen is administered to the subject when the plasma or serum sample obtained from the subject is found to have a statistically different expression level of one or more biomarkers selected from TPO, IL-5, IL-7, I309, IL-6, SAA, PPY, and A2M when compared to the expression level of the corresponding biomarkers from elderly individuals who do not have a family history of memory loss, senility, dementia, or Alzheimer's disease in the statistical sample. In another aspect, naproxen is administered to the subject when the plasma or serum sample obtained from the subject is found to have a statistically similar expression level of one or more biomarkers selected from TPO, IL-5, IL-7, I309, IL-6, SAA, PPY, and A2M when compared to the expression level of the corresponding biomarkers from elderly individuals who have a family history of memory loss, senility, dementia, or Alzheimer's disease in the statistical sample. In one aspect, naproxen is administered to the subject when the plasma or serum sample obtained from the subject is found to have a statistically different expression level of each of TPO, IL-5, IL-7, and I309, and optionally IL-6 when compared to the expression level of the corresponding biomarkers from elderly individuals who do not have a family history of memory loss, senility, dementia, or Alzheimer's disease in the statistical sample. In another aspect, naproxen is administered to the subject when the plasma or serum sample obtained from the subject is found to have a statistically similar expression level of each of TPO, IL-5, IL-7, and I309, and optionally IL-6 when compared to the expression level of the corresponding biomarkers from elderly individuals who have a family history of memory loss, senility, dementia, or Alzheimer's disease in the statistical sample.

In one aspect, the celecoxib or naproxen is administered to the subject in an amount sufficient to improve the subject's cognition or reduce cognitive decline or dysfunction in the subject. In some aspects, celecoxib or naproxen is administered to the subject at a dosage of between about 50-500 mg, 100-450 mg, 100-400 mg, 100-350 mg, 100-300 mg, 100-250 mg, 150-250 mg, or 175-225 mg between once and eight times a day.

In one aspect, the biomarker measurements are obtained by a method selected from the group consisting of an immunoassay, an enzymatic activity assay, fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, and radioactive labeling. In some aspects, the biomarker measurements are obtained by electrochemiluminescence detection.

In some aspects, the method further comprises the step of obtaining the results of one or more neurocognitive evaluations from the untreated subject (i.e. before administration of the NSAID). In some aspects, the method further comprises the steps of obtaining the results of the one or more neurocognitive evaluations from the subject after administration of celecoxib or naproxen and comparing the results of the one or more neurocognitive evaluations before treatment with those after treatment to determine if the NSAID is useful in improving cognition and/or preventing cognitive decline or dysfunction in the subject.

In some aspects, the method further comprises the steps of obtaining a plasma or serum sample from the subject after administration of celecoxib or naproxen and measuring in the sample the expression level of one or more proinflammatory biomarkers selected from the group consisting of IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and I309. In some aspects, a statistically significant change in the expression level of one or more biomarkers when compared to the expression level of the corresponding one or more biomarkers before administration of the NSAID indicates that the NSAID is useful in improving cognition and/or preventing cognitive decline or dysfunction in the subject.

### Method of Diagnosing a Subject as Having a Proinflammatory Endophenotype Profile

In yet another aspect, the present disclosure relates to a method of diagnosing a subject as having a proinflammatory endophenotype profile, the method comprising: obtaining a blood, plasma, or serum sample from the subject; measuring in the sample the expression level of one or more proinflammatory biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY); and determining that the subject has a proinflammatory endophenotype profile based on the expression level of the proinflammatory biomarkers.

The subject can be any subject described elsewhere herein. In one embodiment, the subject is an elderly subject described in the method of selecting a subject for treatment with naproxen or celecoxib or the method of treating a subject with naproxen or celecoxib. The expression level of any biomarkers described elsewhere herein can be measured. In one embodiment, the expression level of one or more of IL-10, eotaxin 3, A2M, SAA, Tenacin C, FABP, IL-5, and TPO is measured. In one embodiment, the expression level of each of IL-10, eotaxin 3, A2M, and SAA, and optionally Tenacin C is measured. In another embodiment, the expression level of one or more of TPO, IL-5, IL-7, I309, IL-6, SAA, PPY, and A2M is measured. In some embodiments, the expression level of each of TPO, IL-5, IL-7, I309, and optionally IL-6 is measured. The expression level of the one or more biomarkers can be measured using any method described elsewhere herein. In one embodiment, the expression level of the one or more biomarkers is measured using electrochemiluminescence. In some embodiments, the expression level of the one or more biomarkers in the blood, plasma, or serum sample obtained from the subject is compared to the expression level of the corresponding one or more biomarkers from a statistical sample representative of the subject. The statistical sample can be any statistical sample described elsewhere herein. In one embodiment, the statistical sample is described in the method of selecting a subject for treatment with naproxen or celecoxib or the method of treating a subject with naproxen or celecoxib. In some embodiments, subjects diagnosed with a proinflammatory endophenotype profile are selected for treatment with an NSAID in order to improve cognition or to prevent cognitive decline or dysfunction in the subject. In one embodiment, the NSAID is celecoxib or naproxen.

### Method of Screening a Subject for Inclusion in an NSAID Clinical Trial

In yet another embodiment, the present disclosure relates to a method of screening a subject for inclusion in an NSAID clinical trial to improve cognition or to prevent cognitive decline or dysfunction in the subject, the method comprising: selecting the subject for screening based on one or more demographic factors or a result of one or more neurocognitive evaluations; obtaining a blood, plasma, or serum sample from the subject; measuring in the sample the expression level of one or more proinflammatory biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY) determining that the subject has a proinflammatory endophenotype profile based on the expression level of the proinflammatory biomarkers; and selecting the subject for inclusion in the NSAID clinical trial.

In one aspect, the subject selected for screening has an elevated risk of age-related memory loss, senility, dementia, Alzheimer's Disease, or a combination thereof due to a family history of one or more of these diseases or disorders. In some aspects, the subject selected for screening is suspected of having age-related memory loss, senility, dementia, or Alzheimer's Disease due to the subject's advanced age and complaints of cognitive decline or dysfunction from the subject or the subject's caretaker.

In one aspect, the sample is a blood sample.

In some aspects, the step of measuring the expression level of the one or more biomarkers in the blood, plasma, or serum sample comprises measuring the expression level of each of IL-10 and eotaxin 3; IL-10, eotaxin 3, and A2M; IL-10, eotaxin 3, A2M, and SAA; IL-10, eotaxin 3, A2M, SAA, and tenacin C; IL-10, eotaxin 3, A2M, SAA, tenacin C, and FABP; IL-10, eotaxin 3, A2M, SAA, and tenacin C; IL-10, eotaxin 3, A2M, SAA, tenacin C, FABP, and IL-5; IL-10, eotaxin 3, A2M, SAA, and tenacin C; IL-10, eotaxin 3, A2M, SAA, tenacin C, FABP, IL-5, and TPO; IL-10, eotaxin 3, A2M, SAA, and tenacin C; IL-10, eotaxin 3, A2M, SAA, tenacin C, FABP, IL-5, TPO, and IL-6; IL-10, eotaxin 3, A2M, SAA, tenacin C, FABP, IL-5, TPO, IL-6, and Factor VII; IL-10, eotaxin 3, A2M, SAA, tenacin C, FABP, IL-5, TPO, IL-6, Factor VII, and IL-18; IL-10, eotaxin 3, A2M, SAA, tenacin C, FABP, IL-5, TPO, IL-6, Factor VII, IL-18, and TARC; IL-10, eotaxin 3, A2M, SAA, tenacin C, FABP, IL-5, TPO, IL-6, Factor VII, IL-18, TARC, and TNFα; IL-10, eotaxin 3, A2M, SAA, tenacin C, FABP, IL-5, TPO, IL-6, Factor VII, IL-18, TARC, TNFα, and sVCAM-1; IL-10, eotaxin 3, A2M, SAA, tenacin C, FABP, IL-5, TPO, IL-6, Factor VII, IL-18, TARC, TNFα, sVCAM-1, and CRP; IL-10, eotaxin 3, A2M, SAA, tenacin C, FABP, IL-5, TPO, IL-6, Factor VII, IL-18, TARC, TNFα, sVCAM-1, CRP, and IL-7; IL-10, eotaxin 3, A2M, SAA, tenacin C, FABP, IL-5, TPO, IL-6, Factor VII, IL-18, TARC, TNFα, sVCAM-1, CRP, IL-7, and sICAM-1; IL-10, eotaxin 3, A2M, SAA, tenacin C, FABP, IL-5, TPO, IL-6, Factor VII, IL-18, TARC, TNFα, sVCAM-1, CRP, IL-7, and sICAM-1; IL-10, eotaxin 3, A2M, SAA, tenacin C, FABP, IL-5, TPO, IL-6, Factor VII, IL-18, TARC, TNFα, sVCAM-1, CRP, IL-7, sICAM-1, and B2M; IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, and PPY; or IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and I309. In some aspects, the subject is being screened for a celecoxib clinical trial and the expression level of one or more of IL-10, eotaxin 3, A2M, SAA, Tenacin C, FABP, IL-5, and TPO are measured in the sample. In one aspect, the subject is being screened for a celecoxib clinical trial and the expression level of each of IL-10, eotaxin 3, A2M, and SAA, and optionally Tenacin C are measured in the sample.

In other aspects, the step of measuring the expression level of the one or more biomarkers in the plasma or serum sample comprises measuring the expression level of each of TPO and IL-5; TPO, IL-5, and IL-7; TPO, IL-5, IL-7, and I309; TPO, IL-5, IL-7, I309, and IL-6; TPO, IL-5, IL-7, I309, IL-6, and SAA; TPO, IL-5, IL-7, I309, IL-6, SAA, and PPY; TPO, IL-5, IL-7, I309, IL-6, SAA, PPY, and A2M; TPO, IL-5, IL-7, I309, IL-6, SAA, PPY, A2M, and TARC; TPO, IL-5, IL-7, I309, IL-6, SAA, PPY, A2M, TARC, and CRP; TPO, IL-5, IL-7, I309, IL-6, SAA, PPY, A2M, TARC, CRP, and sVCAM-1; TPO, IL-5, IL-7, I309, IL-6, SAA, PPY, A2M, TARC, CRP, sVCAM-1, and sICAM-1; TPO, IL-5, IL-7, I309, IL-6, SAA, PPY, A2M, TARC, CRP, sVCAM-1, sICAM-1, and B2M; TPO, IL-5, IL-7, I309, IL-6, SAA, PPY, A2M, TARC, CRP, sVCAM-1, sICAM-1, B2M, and Factor VII; TPO, IL-5, IL-7, I309, IL-6, SAA, PPY, A2M, TARC, CRP, sVCAM-1, sICAM-1, B2M, Factor VII, and IL-10; TPO, IL-5, IL-7, I309, IL-6, SAA, PPY, A2M, TARC, CRP, sVCAM-1, sICAM-1, B2M, Factor VII, IL-10, and tenacin C; TPO, IL-5, IL-7, I309, IL-6, SAA, PPY, A2M, TARC, CRP, sVCAM-1, sICAM-1, B2M, Factor VII, IL-10, tenacin C, and eotaxin 3; TPO, IL-5, IL-7, I309, IL-6, SAA, PPY, A2M, TARC, CRP, sVCAM-1, sICAM-1, B2M, Factor VII, IL-10, tenacin C, eotaxin 3, and IL-18; TPO, IL-5, IL-7, I309, IL-6, SAA, PPY, A2M, TARC, CRP, sVCAM-1, sICAM-1, B2M, Factor VII, IL-10, tenacin C, eotaxin 3, IL-18, and FABP; or TPO, IL-5, IL-7, I309, IL-6, SAA, PPY, A2M, TARC, CRP, sVCAM-1, sICAM-1, B2M, Factor VII, IL-10, tenacin C, eotaxin 3, IL-18, FABP, and TNFα. In some aspects, the subject is being screened for a naproxen clinical trial and the expression level of one or more of TPO, IL-5, IL-7, I309, IL-6, SAA, PPY, and A2M are measured in the sample. In one aspect, the subject is being screened for a naproxen clinical trial and the expression level of each of TPO, IL-5, IL-7, and I309, and optionally IL-6 are measured in the sample.

In one aspect, the proinflammatory endophenotype profile is determined by comparing the expression level of one or more biomarkers in the blood, plasma, or serum sample obtained from the subject to the average expression level of the one or more corresponding biomarkers from a statistical sample representative of the subject. In some aspects, the statistical sample comprises a group of individuals with Alzheimer's disease. In some aspects, the statistical sample comprises a group of individuals 50 years of age or older with Alzheimer's disease. In some aspects, the statistical sample comprises a group of individuals who do not have Alzheimer's disease.

In one aspect, the blood, plasma, or serum sample obtained from the subject has a statistically different expression level of each of IL-10, eotaxin 3, A2M, and SAA, and optionally Tenacin C when compared to a group of individuals in the statistical sample who do not have Alzheimer's disease. In one aspect, this statistically different expression level indicates that the subject should be selected for a celecoxib clinical trial. In another aspect, the blood, plasma, or serum sample obtained from the subject has a statistically similar expression level of each of IL-10, eotaxin 3, A2M, and SAA, and optionally Tenacin C when compared to a group of individuals in the statistical sample who have Alzheimer's disease. In one aspect, this statistically similar expression level indicates that the subject should be selected for a celecoxib clinical trial. In another aspect, the blood, plasma, or serum sample obtained from the subject has a statistically different expression level of each of TPO, IL-5, IL-7, and I309, and optionally IL-6 when compared to a group of individuals in the statistical sample who do not have Alzheimer's disease. In some aspects, the statistically different expression level indicates that the subject should be selected for a naproxen clinical trial. In yet another aspect, the blood, plasma, or serum sample obtained from the subject has a statistically similar expression level of each of TPO, IL-7, and I309, and optionally IL-6 when compared to a group of individuals in the statistical sample who have Alzheimer's disease. In one aspect, this statistically similar expression level indicates that the subject should be selected for a naproxen clinical trial.

In one aspect, the one or more demographic factors are selected from the group consisting of age, education level, APOE ε4 allele frequency, and combinations thereof. In some aspects, the age, education level, APOE ε4 allele frequency or a combination thereof is compared to the corresponding one or more demographic factors from a statistical sample representative of the subject. In some aspects, the statistical sample comprises a group of individuals with Alzheimer's disease. In some aspects, the statistical sample comprises a group of individuals 50 years of age or older with Alzheimer's disease. In some aspects, the statistical sample comprises a group of individuals who do not have Alzheimer's disease.

In one aspect, the age, education level, or APOE ε4 allele frequency of the subject is statistically similar to individuals in the statistical sample who do not have Alzheimer's disease. In some aspects, this statistically similar demographic factor indicates that the subject should not undergo further screening for the NSAID clinical trial. In another aspect, the age of the subject is statistically lower than individuals in the statistical sample who do not have Alzheimer's disease, the education level of the subject is statistically higher than individuals in the statistical sample who do not have Alzheimer's disease, and/or the subject does not have an APOE ε4 allele, and the subject does not undergo further screening for the NSAID clinical trial. In another aspect, the age, education level, or APOE ε4 allele frequency of the subject is statistically similar to individuals in the statistical sample who have Alzheimer's disease. In some aspects, this statistically similar demographic factor indicates that the subject should undergo further screening for the NSAID clinical trial. In yet another aspect, the age of the subject is statistically higher than individuals in the statistical sample who have Alzheimer's disease, the education level of the subject is statistically lower than individuals in the statistical sample who have Alzheimer's disease, and/or the subject has one or more APOE ε4 alleles, and the subject undergoes further screening for the NSAID agonist clinical trial.

In one aspect, the one or more neurocognitive evaluations are selected from the group consisting of a 4-point clock drawing test, a verbal fluency test, a trail making test, a list learning test, a mini-mental state exam, and combinations thereof. In some aspects, the results of the one or more neurocognitive evaluations of the subject are compared to the corresponding one or more neurocognitive evaluations from a statistical sample representative of the subject. In some aspects, the statistical sample comprises a group of individuals with Alzheimer's disease. In some aspects, the statistical sample comprises a group of individuals 50 years of age or older with Alzheimer's disease. In some aspects, the statistical sample comprises a group of individuals who do not have Alzheimer's disease. In one aspect, the results of the one or more neurocognitive evaluations of the subject are statistically similar to individuals in the statistical sample who have Alzheimer's disease. In one aspect, this statistically similar neurocognitive evaluation result indicates that the subject should undergo further screening for the NSAID clinical trial. In one aspect, the results of the one or more neurocognitive evaluations of the subject are statistically different from individuals in the statistical sample who do not have Alzheimer's disease. In one aspect, this statistically different neurocognitive evaluation result indicates that the subject should undergo further screening for the NSAID clinical trial. In yet another aspect, the results of the one or more neurocognitive evaluations of the subject are statistically different from individuals in the statistical sample who have Alzheimer's disease. In one aspect, this statistically different neurocognitive evaluation result indicates that the subject should not undergo further screening for the NSAID clinical trial. In yet another aspect, the results of the one or more neurocognitive evaluations of the subject are statistically similar to individuals in the statistical sample who do not have Alzheimer's disease. In one aspect, this statistically different neurocognitive evaluation result indicates that the subject should not undergo further screening for the NSAID clinical trial.

In one aspect, the NSAID is selected from celecoxib and naproxen.

### Method of Determining a Surrogate Outcome of an NSAID Clinical Trial

In yet another embodiment, the present disclosure relates to a method of determining a surrogate outcome of an NSAID clinical trial to improve cognition or to prevent cognitive decline or dysfunction, the method comprising: (a) selecting a subject to participate in the clinical trial, wherein the subject is suspected of having age-related memory loss, senility, dementia, or Alzheimer's Disease, or having an elevated risk of age-related memory loss, senility, dementia, or Alzheimer's Disease; (b) measuring in a plasma or serum sample obtained from the subject the expression level of one or more biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY); (c) administering an NSAID selected from celecoxib and naproxen to a first subset of subjects and a placebo to a second subset of subjects; (d) repeating step (b) after the administration of the NSAID or the placebo; and (e) determining if a statistically significant change in the expression level of the one or more biomarkers is seen in the first subset of subjects when compared to any change occurring in the second subset of subjects, wherein a statistically significant change in the expression level is associated with a beneficial long term clinical outcome.

In one aspect, the subject selected for the clinical trial has an elevated risk of age-related memory loss, senility, dementia, Alzheimer's Disease, or a combination thereof due to a family history of one or more of these diseases or disorders. In one aspect, the subject selected for the clinical trial is an elderly subject. In some aspects, the subject selected for the clinical trial is suspected of having age-related memory loss, senility, dementia, or Alzheimer's Disease due to the subject's advanced age and complaints of cognitive decline or dysfunction from the subject or the subject's caretaker. In another aspect, the subject selected for the clinical trial is suspected of having age-related memory loss, senility, dementia, or Alzheimer's Disease due to the results of one or more neurocognitive evaluations. Exemplary neurocognitive evaluations are described elsewhere herein. In one aspect, the subject has a proinflammatory endophenotype profile.

In one aspect, the sample is a blood sample.

In one aspect, in steps (b) and (d), the expression level of each biomarker selected from the group consisting of IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and I309 is measured.

In one aspect wherein the NSAID administered in the clinical trial is celecoxib, steps (b) and (d) comprise measuring the expression level of each of IL-10 and eotaxin 3; IL-10, eotaxin 3, and A2M; IL-10, eotaxin 3, A2M, and SAA; IL-10, eotaxin 3, A2M, SAA, and tenacin C; IL-10, eotaxin 3, A2M, SAA, tenacin C, and FABP; IL-10, eotaxin 3, A2M, SAA, and tenacin C; IL-10, eotaxin 3, A2M, SAA, tenacin C, FABP, and IL-5; IL-10, eotaxin 3, A2M, SAA, and tenacin C; IL-10, eotaxin 3, A2M, SAA, tenacin C, FABP, IL-5, and TPO; IL-10, eotaxin 3, A2M, SAA, and tenacin C; IL-10, eotaxin 3, A2M, SAA, tenacin C, FABP, IL-5, TPO, and IL-6; IL-10, eotaxin 3, A2M, SAA, tenacin C, FABP, IL-5, TPO, IL-6, and Factor VII; IL-10, eotaxin 3, A2M, SAA, tenacin C, FABP, IL-5, TPO, IL-6, Factor VII, and IL-18; IL-10, eotaxin 3, A2M, SAA, tenacin C, FABP, IL-5, TPO, IL-6, Factor VII, IL-18, and TARC; IL-10, eotaxin 3, A2M, SAA, tenacin C, FABP, IL-5, TPO, IL-6, Factor VII, IL-18, TARC, and TNFα; IL-10, eotaxin 3, A2M, SAA, tenacin C, FABP, IL-5, TPO, IL-6, Factor VII, IL-18, TARC, TNFα, and sVCAM-1; IL-10, eotaxin 3, A2M, SAA, tenacin C, FABP, IL-5, TPO, IL-6, Factor VII, IL-18, TARC, TNFα, sVCAM-1, and CRP; IL-10, eotaxin 3, A2M, SAA, tenacin C, FABP, IL-5, TPO, IL-6, Factor VII, IL-18, TARC, TNFα, sVCAM-1, CRP, and IL-7; IL-10, eotaxin 3, A2M, SAA, tenacin C, FABP, IL-5, TPO, IL-6, Factor VII, IL-18, TARC, TNFα, sVCAM-1, CRP, IL-7, and sICAM-1; IL-10, eotaxin 3, A2M, SAA, tenacin C, FABP, IL-5, TPO, IL-6, Factor VII, IL-18, TARC, TNFα, sVCAM-1, CRP, IL-7, and sICAM-1; IL-10, eotaxin 3, A2M, SAA, tenacin C, FABP, IL-5, TPO, IL-6, Factor VII, IL-18, TARC, TNFα, sVCAM-1, CRP, IL-7, sICAM-1, and B2M; IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, and PPY; or IL-10, eotaxin 3, A2M, SAA, tenacin C, FABP, IL-5, TPO, IL-6, Factor VII, IL-18, TARC, TNFα, sVCAM-1, CRP, IL-7, sICAM-1, B2M, PPY, and 1309.

In some aspects, wherein the NSAID administered in the clinical trial is celecoxib, the expression level of one or more biomarkers selected from the group consisting of FABP, tenacin C, B2M, TPO, IL-5, sVCAM-1, CRP, and IL-10 is measured in steps (b) and (d). In one aspect wherein the NSAID administered in the clinical trial is celecoxib, the expression level of one or more biomarkers selected from the group consisting of FABP, tenacin C, B2M, TPO, and IL-5 is measured in steps (b) and (d). In some aspects wherein the NSAID administered in the clinical trial is celecoxib, the expression level of each of the biomarkers FABP, tenacin C, B2M, and TPO, and optionally IL-5 is measured in steps (b) and (d).

In other aspects, wherein the NSAID administered in the clinical trial is napoxen, steps (b) and (d) comprise measuring the expression level of each of TPO and IL-5; TPO, IL-5, and IL-7; TPO, IL-5, IL-7, and I309; TPO, IL-5, IL-7, I309, and IL-6; TPO, IL-5, IL-7, I309, IL-6, and SAA; TPO, IL-5, IL-7, I309, IL-6, SAA, and PPY; TPO, IL-5, IL-7, I309, IL-6, SAA, PPY, and A2M; TPO, IL-5, IL-7, I309, IL-6, SAA, PPY, A2M, and TARC; TPO, IL-5, IL-7, I309, IL-6, SAA, PPY, A2M, TARC, and CRP; TPO, IL-5, IL-7, I309, IL-6, SAA, PPY, A2M, TARC, CRP, and sVCAM-1; TPO, IL-5, IL-7, I309, IL-6, SAA, PPY, A2M, TARC, CRP, sVCAM-1, and sICAM-1; TPO, IL-5, IL-7, I309, IL-6, SAA, PPY, A2M, TARC, CRP, sVCAM-1, sICAM-1, and B2M; TPO, IL-5, IL-7, I309, IL-6, SAA, PPY, A2M, TARC, CRP, sVCAM-1, sICAM-1, B2M, and Factor VII; TPO, IL-5, IL-7, I309, IL-6, SAA, PPY, A2M, TARC, CRP, sVCAM-1, sICAM-1, B2M, Factor VII, and IL-10; TPO, IL-5, IL-7, I309, IL-6, SAA, PPY, A2M, TARC, CRP, sVCAM-1, sICAM-1, B2M, Factor VII, IL-10, and tenacin C; TPO, IL-5, IL-7, I309, IL-6, SAA, PPY, A2M, TARC, CRP, sVCAM-1, sICAM-1, B2M, Factor VII, IL-10, tenacin C, and eotaxin 3; TPO, IL-5, IL-7, I309, IL-6, SAA, PPY, A2M, TARC, CRP, sVCAM-1, sICAM-1, B2M, Factor VII, IL-10, tenacin C, eotaxin 3, and IL-18; TPO, IL-5, IL-7, I309, IL-6, SAA, PPY, A2M, TARC, CRP, sVCAM-1, sICAM-1, B2M, Factor VII, IL-10, tenacin C, eotaxin 3, IL-18, and FABP; or TPO, IL-5, IL-7, I309, IL-6, SAA, PPY, A2M, TARC, CRP, sVCAM-1, sICAM-1, B2M, Factor VII, IL-10, tenacin C, eotaxin 3, IL-18, FABP, and TNFα.

In another aspect wherein the NSAID administered in the clinical trial is naproxen, the expression level of one or more biomarkers selected from the group consisting of TPO, IL-6, CRP, FABP, Factor VII, IL-5, SAA, and A2M is measured in steps (b) and (d). In another aspect wherein the NSAID administered in the clinical trial is naproxen, the expression level of one or more biomarkers selected from the group consisting of TPO, IL-6, CRP, FABP, and Factor VII is measured in steps (b) and (d). In some aspects wherein the NSAID administered in the clinical trial is naproxen, the expression level of each of the biomarkers TPO, IL-6, CRP, and FABP, and optionally Factor VII is measured in steps (b) and (d). In some aspects, a statistically significant reduction in the expression level of one or more of the biomarkers measured in step (d) compared to the expression level of the corresponding biomarker in step (b) indicates that the NSAID is useful in improving cognition or preventing cognitive decline or dysfunction and is associated with a beneficial long term clinical outcome. In other aspects, a statistically significant increase in the expression level of one or more of the biomarkers measured in step (d) compared to the expression level of the corresponding biomarker in step (b) indicates that the NSAID is useful in improving cognition or preventing cognitive decline or dysfunction and is associated with a beneficial long term clinical outcome. In some aspects, step (d) occurs about one or more months after step (c). In one aspect, step (d) occurs about six months after step (c). In another aspect, step (d) occurs about twelve months after step (c). In one aspect, a statistically significant change in the expression level in step (d) at about twelve months after (c) is associated with a beneficial clinical outcome in about 24 months, about 36 months, about 48 months, or about 60 months. Therefore, in one aspect, a statistically significant change in the expression level in step (d) at about twelve months after (c) provides a surrogate outcome of the clinical trial at about 24 months, about 36 months, about 48 months, or about 60 months.

In one aspect, the NSAID is administered to the first subset of subjects during the clinical trial in an amount believed to be sufficient to determine if the NSAID is useful to improve cognition or reduce cognitive decline or dysfunction in the subject. In some aspects, the NSAID is administered to the first subset of subjects at a dosage of between about 50-500 mg, 100-450 mg, 100-400 mg, 100-350 mg, 100-300 mg, 100-250 mg, 150-250 mg, or 175-225 mg between once and eight times a day.

In one aspect, the biomarker measurements in steps (b) and (c) are obtained from an immunoassay, an enzymatic activity assay, fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In one aspect, the biomarker measurements in steps (b) and (c) are obtained from a electrochemiluminescence detection.

In one aspect, the method further comprises the steps of obtaining the results of the one or more neurocognitive evaluations from the first subset and second subset of subjects before step (c), obtaining the results of the corresponding one or more neurocognitive evaluations from the first subset and second subset of subjects after step (c), and determining if the first subset of subjects demonstrated a statistically significant improvement in the one or more neurocognitive evaluations when compared to any improvement occurring in the second subset of subjects, wherein a statistically significant improvement in the one or more neurocognitive evaluations and a statistically significant change in the expression level of one or more biomarkers is associated with a beneficial long term clinical outcome.

In one aspect, the method further comprises step (f) of terminating the clinical trial when a surrogate outcome has been determined. In some aspects, a statistically significant change in the expression level of the one or more biomarkers is seen in the first subset of subjects when compared to any change occurring in the second subset of subjects indicates that a surrogate outcome of the trial has been determined. In some aspects, a statistically significant change in the expression level of the one or more biomarkers is seen in the first subset of subjects when compared to any change occurring in the second subset of subjects indicates that the clinical trial is successful in treating a subject suspected of having age-related memory loss, senility, dementia, or Alzheimer's Disease, or having an elevated risk of age-related memory loss, senility, dementia, or Alzheimer's Disease.

Method of Selecting an AD Patient for Treatment with a PPAR-γ Agonist

In yet another embodiment, the present disclosure provides a method of selecting an Alzheimer's disease patient for treatment with a PPAR-γ agonist, the method comprising: obtaining a plasma or serum sample from the patient; measuring in the sample the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (FVII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol; determining that the patient has both a proinflammatory endophenotype profile and a metabolic endophenotype profile based on the expression level of the one or more biomarkers; and selecting the patient for treatment with a PPAR-γ agonist.

In one aspect, the patient is an elderly patient. In some aspects, the patient is 50 years of age or older. In some aspects, the patient has been previously diagnosed with mild to moderate Alzheimer's disease.

In one aspect, the expression level of one or more biomarkers selected from IL-7, TNFα, IL-5, IL-6, CRP, IL-10, tenascin C, ICAM1, Factor VII, I309, TNFR1, A2M, TARC, eotaxin3, VCAM1, TPO, FABP, IL-18, B2M, SAA, PPY, DJ1, Aβ, tau, α-synuclein, GLP-1), PYY, insulin, HbA1c, glucose, triglycerides, HDL, LDL, vLDL, DGAT1, PPAR-γ, PPARα, and cholesterol in the plasma or serum sample is compared to the average level of expression of the one or more corresponding biomarkers in a statistical sample representative of the patient. In some aspects, the statistical sample comprises individuals with Alzheimer's disease. In some aspects, the statistical sample comprises individuals 50 years of age or older with Alzheimer's disease. In some aspects, the statistical sample comprises individuals 50 years of age or older who do not have Alzheimer's disease. In some aspects, the statistical sample comprises the measurements of the expression level for one or more biomarkers selected from IL-7, TNFα, IL-5, IL-6, CRP, IL-10, tenascin C, ICAM1, Factor VII, I309, TNFR1, A2M, TARC, eotaxin3, VCAM1, TPO, FABP, IL-18, B2M, SAA, PPY, DJ1, Aβ, tau, α-synuclein, GLP-1), PYY, insulin, HbA1c, glucose, triglycerides, HDL, LDL, vLDL, DGAT1, PPAR-γ, PPARα, and cholesterol from each individual in the statistical sample.

In one aspect, the expression level of one or more biomarkers selected from IL-6, IL-10, CRP, TNFα, FABP, and PPY in the plasma or serum sample is measured. In one aspect, the expression level of each of IL-6, IL-10, CRP, TNFα, FABP, and PPY in the plasma or serum sample is measured. In one aspect, the plasma or serum sample obtained from the patient is found to have a statistically different expression level of one or more biomarkers when compared to the corresponding one or more biomarkers from individuals who have been diagnosed with AD in statistical sample. In some aspects, this statistically different expression level when compared to individuals who have been diagnosed with AD in statistical sample indicates that the patient does not have a proinflammatory endophenotype profile and a metabolic endophenotype profile and the patient is not selected for treatment with a PPAR-γ agonist. In another aspect, the plasma or serum sample obtained from the patient is found to have a statistically similar expression level of one or more biomarkers when compared to the corresponding one or more biomarkers from individuals who have been diagnosed with AD in statistical sample. In some aspects, this statistically similar expression level when compared to individuals who have been diagnosed with AD in statistical sample indicates that the patient has a proinflammatory endophenotype profile and a metabolic endophenotype profile and the patient is selected for treatment with a PPAR-γ agonist. In yet another aspect, the plasma or serum sample obtained from the patient is found to have a statistically different expression level of one or more biomarkers when compared to the corresponding one or more biomarkers from individuals who do not have AD in statistical sample. In some aspects, this statistically different expression level when compared to individuals who do not have AD in statistical sample indicates that the patient has a proinflammatory endophenotype profile and a metabolic endophenotype profile and the patient is selected for treatment with a PPAR-γ agonist.

In one aspect, the plasma or serum sample obtained from the patient is found to have an elevated expression level of one or more biomarkers when compared to a “type” of individuals the statistical sample (e.g. when compared to either individuals diagnosed with AD or individuals who do not have AD). In another aspect, the plasma or serum sample obtained from the patient is found to have an reduced expression level of one or more biomarkers when compared to a “type” of individuals in the statistical sample. In one aspect, an elevated expression level of one or more biomarkers selected from IL-6, IL-10, CRP, and TNFα and an elevated expression level of FABP and/or PPY indicates that the patient has a proinflammatory endophenotype profile and a metabolic endophenotype profile. In some aspects, an elevated expression level of each of IL-6, IL-10, CRP, TNFα, FABP, and PPY when compared to a “type” of individuals in the statistical sample indicates that the patient has a proinflammatory endophenotype profile and a metabolic endophenotype profile. In another aspect, a reduced expression level of one or more biomarkers selected from IL-6, IL-10, CRP, and TNFα and a reduced expression level of FABP and/or PPY when compared to a “type” of individuals in the statistical sample indicates that the patient has a proinflammatory endophenotype profile and a metabolic endophenotype profile. In some aspects, a reduced expression level of each of IL-6, IL-10, CRP, TNFα, FABP, and PPY when compared to a “type” of individuals in the statistical sample indicates that the patient has a proinflammatory endophenotype profile and a metabolic endophenotype profile. In another aspect, a reduced expression level of one or more biomarkers selected from IL-6, IL-10, CRP, and TNFα and an elevated expression level of FABP and/or PPY when compared to a “type” of individuals in the statistical sample indicates that the patient has a proinflammatory endophenotype profile and a metabolic endophenotype profile. In some aspects, a reduced expression level of each of IL-6, IL-10, CRP, and TNFα and an elevated expression level of each of FABP and PPY when compared to a “type” of individuals in the statistical sample indicates that the patient has an inflammatory endophenotype profile and a metabolic endophenotype profile. In another aspect, an elevated expression level of one or more biomarkers selected from IL-6, IL-10, CRP, and TNFα and a reduced expression level of FABP and/or PPY when compared to a “type” of individuals in the statistical sample indicates that the patient has a proinflammatory endophenotype profile and a metabolic endophenotype profile. In some aspects, an elevated expression level of each of IL-6, IL-10, CRP, and TNFα and a reduced expression level of each of FABP and PPY when compared to a “type” of individuals in the statistical sample indicates that the patient has a proinflammatory endophenotype profile and a metabolic endophenotype profile. In some aspects, a patient determined to have a proinflammatory endophenotype profile and a metabolic endophenotype profile is classified as a responder or a stable responder to the PPAR-γ agonist and therefore the patient is selected for treatment with the PPAR-γ agonist. In another aspect, a patient who does not have a proinflammatory endophenotype profile and a metabolic endophenotype profile is classified as a non-responder or an adverse responder to the PPAR-γ agonist and therefore the patient is not selected for treatment with the PPAR-γ agonist.

In one aspect, the biomarker measurements are obtained by a method selected from the group consisting of an immunoassay, an enzymatic activity assay, fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, and radioactive labeling. In some aspects, the biomarker measurements are obtained by electrochemiluminescence detection.

The PPAR-γ agonist can be any PPAR-γ agonist known to a person of skill in the art. In one aspect, the PPAR-γ agonist is rosiglitazone. In another aspect, the PPAR-γ agonist is rosiglitazone XR.

In some aspects, the method further comprises the steps of measuring the body mass index (BMI) and/or waist circumference of the patient and determining if the BMI and/or waist circumference of the patient is statistically different than the average BMI and/or waist circumference obtained from a statistical sample representative of the patient. In some aspects, the statistical sample comprises a group of individuals with Alzheimer's disease. In some aspects, the statistical sample comprises a group of individuals 50 years of age or older with Alzheimer's disease. In some aspects, the statistical sample comprises a group of individuals who do not have Alzheimer's disease. In some aspects, the statistical sample comprises the BMI and/or waist circumference measurements of each individual in the statistical sample. In one aspect, a statistically significant increase in BMI and/or waist circumference in the patient when compared to either the group of individuals in the statistical sample who do not have AD and/or the group of individuals who do have AD indicates that the patient may have a metabolic endophenotype profile. Therefore, a statistically significant increase in BMI and/or waist circumference can be used in conjunction with the expression level of the one or more biomarkers to determine if the patient has a proinflammatory endophenotype profile and a metabolic endophenotype profile. In some aspects, a statistically significant increase in BMI and/or waist circumference and the statistically different expression level of one or more biomarkers selected from the group consisting of IL-6, IL-10, CRP, TNFα, FABP, and PPY when compared to the statistical sample can be used to determine if the patient has a proinflammatory endophenotype profile and a metabolic endophenotype profile.

Method of Treating an AD Patient with a PPAR-γ Agonist

In yet another embodiment, the present disclosure provides a method of treating an Alzheimer's disease patient to improve cognition or to prevent cognitive decline or dysfunction in the patient, the method comprising: obtaining a plasma or serum sample from the patient; measuring in the sample the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol; determining that the patient has both a proinflammatory endophenotype profile and a metabolic endophenotype profile based on the expression level of the one or more biomarkers; and administering a PPAR-γ agonist to the patient.

In one aspect, the patient is an elderly patient. In some aspects, the patient is 50 years of age or older. In some aspects, the patient has been previously diagnosed with mild to moderate Alzheimer's disease. In some aspects, the patient being treated with an acetylcholinesterase inhibitor (AChEI). In one aspect, the patient is being treated with donepezil. In some aspects, administration of the AChEI to the patient is continued during the step of administering a PPAR-γ agonist to the patient.

In one aspect, the expression level of one or more biomarkers selected from IL-7, TNFα, IL-5, IL-6, CRP, IL-10, tenascin C, ICAM1, Factor VII, I309, TNFR1, A2M, TARC, eotaxin3, VCAM1, TPO, FABP, IL-18, B2M, SAA, PPY, DJ1, Aβ, tau, α-synuclein, GLP-1), PYY, insulin, HbA1c, glucose, triglycerides, HDL, LDL, vLDL, DGAT1, PPAR-γ, PPARα, and cholesterol in the plasma or serum sample is compared to the average level of expression of the one or more corresponding biomarkers in a statistical sample representative of the patient. In some aspects, the statistical sample comprises individuals with Alzheimer's disease. In some aspects, the statistical sample comprises individuals 50 years of age or older with Alzheimer's disease. In some aspects, the statistical sample comprises individuals 50 years of age or older who do not have Alzheimer's disease. In some aspects, the statistical sample comprises the measurements of the expression level for one or more biomarkers selected from IL-7, TNFα, IL-5, IL-6, CRP, IL-10, tenascin C, ICAM1, Factor VII, I309, TNFR1, A2M, TARC, eotaxin3, VCAM1, TPO, FABP, IL-18, B2M, SAA, PPY, DJ1, Aβ, tau, α-synuclein, GLP-1), PYY, insulin, HbA1c, glucose, triglycerides, HDL, LDL, vLDL, DGAT1, PPAR-γ, PPARα, and cholesterol from each individual in the statistical sample.

In one aspect, the expression level of one or more biomarkers selected from IL-6, IL-10, CRP, TNFα, FABP, and PPY in the plasma or serum sample is measured. In one aspect, the expression level of each of IL-6, IL-10, CRP, TNFα, FABP, and PPY in the plasma or serum sample is measured. In one aspect, the plasma or serum sample obtained from the patient is found to have a statistically different expression level of one or more biomarkers when compared to the corresponding one or more biomarkers from individuals who have been diagnosed with AD in statistical sample. In some aspects, this statistically different expression level when compared to individuals who have been diagnosed with AD in statistical sample indicates that the patient does not have a proinflammatory endophenotype profile and a metabolic endophenotype profile and the patient a PPAR-γ agonist is not administered to the patient. In another aspect, the plasma or serum sample obtained from the patient is found to have a statistically similar expression level of one or more biomarkers when compared to the corresponding one or more biomarkers from individuals who have been diagnosed with AD in statistical sample. In some aspects, this statistically similar expression level when compared to individuals who have been diagnosed with AD in statistical sample indicates that the patient has a proinflammatory endophenotype profile and a metabolic endophenotype profile and a PPAR-γ agonist is administered to the patient. In yet another aspect, the plasma or serum sample obtained from the patient is found to have a statistically different expression level of one or more biomarkers when compared to the corresponding one or more biomarkers from individuals who do not have AD in statistical sample. In some aspects, this statistically different expression level when compared to individuals who do not have AD in statistical sample indicates that the patient has a proinflammatory endophenotype profile and a metabolic endophenotype profile and a PPAR-γ agonist is administered to the patient.

In one aspect, the plasma or serum sample obtained from the patient is found to have an elevated expression level of one or more biomarkers when compared to a “type” of individuals the statistical sample (e.g. when compared to either individuals diagnosed with AD or individuals who do not have AD). In another aspect, the plasma or serum sample obtained from the patient is found to have a reduced expression level of one or more biomarkers when compared to a “type” of individuals in the statistical sample. In one aspect, an elevated expression level of one or more biomarkers selected from IL-6, IL-10, CRP, and TNFα and an elevated expression level of FABP and/or PPY indicates that the patient has a proinflammatory endophenotype profile and a metabolic endophenotype profile. In some aspects, an elevated expression level of each of IL-6, IL-10, CRP, TNFα, FABP, and PPY when compared to a “type” of individuals in the statistical sample indicates that the patient has a proinflammatory endophenotype profile and a metabolic endophenotype profile. In another aspect, a reduced expression level of one or more biomarkers selected from IL-6, IL-10, CRP, and TNFα and a reduced expression level of FABP and/or PPY when compared to a “type” of individuals in the statistical sample indicates that the patient has a proinflammatory endophenotype profile and a metabolic endophenotype profile. In some aspects, a reduced expression level of each of IL-6, IL-10, CRP, TNFα, FABP, and PPY when compared to a “type” of individuals in the statistical sample indicates that the patient has a proinflammatory endophenotype profile and a metabolic endophenotype profile. In another aspect, a reduced expression level of one or more biomarkers selected from IL-6, IL-10, CRP, and TNFα and an elevated expression level of FABP and/or PPY when compared to a “type” of individuals in the statistical sample indicates that the patient has a proinflammatory endophenotype profile and a metabolic endophenotype profile. In some aspects, a reduced expression level of each of IL-6, IL-10, CRP, and TNFα and an elevated expression level of each of FABP and PPY when compared to a “type” of individuals in the statistical sample indicates that the patient has an inflammatory endophenotype profile and a metabolic endophenotype profile. In another aspect, an elevated expression level of one or more biomarkers selected from IL-6, IL-10, CRP, and TNFα and a reduced expression level of FABP and/or PPY when compared to a “type” of individuals in the statistical sample indicates that the patient has a proinflammatory endophenotype profile and a metabolic endophenotype profile. In some aspects, an elevated expression level of each of IL-6, IL-10, CRP, and TNFα and a reduced expression level of each of FABP and PPY when compared to a “type” of individuals in the statistical sample indicates that the patient has a proinflammatory endophenotype profile and a metabolic endophenotype profile. In some aspects, a patient who does not have a proinflammatory endophenotype profile and a metabolic endophenotype is not administered a PPAR-γ agonist.

In one aspect, the biomarker measurements are obtained by a method selected from the group consisting of an immunoassay, an enzymatic activity assay, fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, and radioactive labeling. In some aspects, the biomarker measurements are obtained by electrochemiluminescence detection.

The PPAR-γ agonist can be any PPAR-γ agonist known to a person of skill in the art. In one aspect, the PPAR-γ agonist is rosiglitazone. In another aspect, the PPAR-γ agonist is rosiglitazone XR. In one aspect, the PPAR-γ agonist is administered to the patient in an amount sufficient to improve the patient's cognition or reduce cognitive decline or dysfunction in the patient. In some aspects, rosiglitazone or rosiglitazone XR is administered to the patient at a dosage of between about 0.5-20 mg, about 0.5-18 mg, about 0.5-16 mg, about 0.5-14 mg, about 0.5-12 mg, about 1.0-12 mg, about 2-10 mg, between once and eight times a day. In one aspect, about 2 mg, about 4 mg, or about 8 mg of rosiglitazone or rosiglitazone XR is administered to the patient once a day. In some aspects, the patient is first administered about 2 mg rosiglitazone or rosiglitazone XR, the patient is evaluated for improved cognition and/or the prevention of cognitive decline or dysfunction, and the dosage of rosiglitazone or rosiglitazone XR is increased if the patient has not improved.

In some aspects, the method further comprises the steps of measuring the body mass index (BMI) and/or waist circumference of the patient and determining if the BMI and/or waist circumference of the patient is statistically different than the average BMI and/or waist circumference obtained from a statistical sample representative of the patient. In some aspects, the statistical sample comprises a group of individuals with Alzheimer's disease. In some aspects, the statistical sample comprises a group of individuals 50 years of age or older with Alzheimer's disease. In some aspects, the statistical sample comprises a group of individuals who do not have Alzheimer's disease. In some aspects, the statistical sample comprises the BMI and/or waist circumference measurements of each individual in the statistical sample. In one aspect, a statistically significant increase in BMI and/or waist circumference in the patient when compared to either the group of individuals in the statistical sample who do not have AD and/or the group of individuals who do have AD indicates that the patient may have a metabolic endophenotype profile. Therefore, a statistically significant increase in BMI and/or waist circumference can be used in conjunction with the expression level of the one or more biomarkers to determine if the patient has a proinflammatory endophenotype profile and a metabolic endophenotype profile. In some aspects, a statistically significant increase in BMI and/or waist circumference and the statistically different expression level of one or more biomarkers selected from the group consisting of IL-6, IL-10, CRP, TNFα, FABP, and PPY when compared to the statistical sample can be used to determine if the patient has a proinflammatory endophenotype profile and a metabolic endophenotype profile.

In some aspects, the method further comprises the step of obtaining the results of one or more neurocognitive evaluations from the patient before administration of a PPAR-γ agonist. In some aspects, the method further comprises the steps of obtaining the results of the one or more neurocognitive evaluations from the patient after administration of a PPAR-γ agonist and comparing the results of the one or more neurocognitive evaluations before treatment with those after treatment to determine if the PPAR-γ agonist is useful in improving cognition and/or preventing cognitive decline or dysfunction in the patient.

### Method of Diagnosing a Subject as Having a Combination Proinflammatory and Metabolic Endophenotype Profile

In yet another aspect, the present disclosure relates to a method of diagnosing a subject as having an endophenotype profile that is a combination of a proinflammatory endophenotype profile and a metabolic endophenotype profile, the method comprising: obtaining a plasma or serum sample from the subject; measuring in the sample the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol; and determining that the subject has both a proinflammatory endophenotype profile and a metabolic endophenotype profile based on the expression level of the one or more biomarkers.

The subject can be any subject described elsewhere herein. In one embodiment, the subject is an elderly Alzheimer's disease patient described in the method of selecting a subject for treatment with a PPAR-γ agonist or the method of treating a subject with a PPAR-γ agonist. The expression level of any biomarkers described elsewhere herein can be measured. In one embodiment, the expression level of one or more of IL-6, IL-10, CRP, TNFα, FABP, and PPY is measured. In another embodiment, the expression level of one or more of IL-6, IL-10, CRP, TNFα and the expression level of one or more of FABP and PPY is measured. In another embodiment, the expression level of each of IL-6, IL-10, CRP, TNFα, FABP, and PPY is measured. The expression level of the one or more biomarkers can be measured using any method described elsewhere herein. In one embodiment, the expression level of the one or more biomarkers is measured using electrochemiluminescence. In some embodiments, the expression level of the one or more biomarkers in the blood, plasma, or serum sample obtained from the subject is compared to the expression level of the corresponding one or more biomarkers from a statistical sample representative of the subject. The statistical sample can be any statistical sample described elsewhere herein. In one embodiment, wherein the subject is an Alzheimer's disease patient, the statistical sample is described in the method of selecting a subject for treatment with a PPAR-γ agonist or the method of treating a subject with a PPAR-γ agonist. In one embodiment, the method further comprises the step of measuring the BMI and/or waist circumference of the subject. In some embodiments, the BMI and/or waist circumference of the subject is compared to the BMI and/or waist circumference measurements from a statistical sample representative of the subject. The statistical sample can be any statistical sample described elsewhere herein. In one embodiment wherein the subject is an Alzheimer's disease patient, the statistical sample is described in the method of selecting a subject for treatment with a PPAR-γ agonist or the method of treating a subject with a PPAR-γ agonist. In some embodiments, subjects diagnosed with both a proinflammatory endophenotype profile and a metabolic endophenotype profile are selected for treatment with a PPAR-γ agonist in order to improve cognition or to prevent cognitive decline or dysfunction in the subject. In one embodiment, the cognitive decline or dysfunction is related to memory loss, senility, dementia, Alzheimer's disease, or a combination thereof.

### Method of Screening a Subject for Inclusion in a PPAR-γ Agonist Clinical Trial

In yet another embodiment, the present disclosure relates to a method of screening a subject for inclusion in a PPAR-γ agonist clinical trial to improve cognition or to prevent cognitive decline or dysfunction in the subject, the method comprising: (a) selecting the subject for screening based on one or more demographic factors or a result of one or more neurocognitive evaluations; (b) obtaining a blood, plasma, or serum sample from the subject; (c) measuring in the sample the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol; (d) determining that the subject has both a proinflammatory endophenotype profile and a metabolic endophenotype profile based on the expression level of the one or more biomarkers; and (e) selecting the subject for inclusion in the PPAR-γ agonist clinical trial.

In one aspect, the subject selected for screening has an elevated risk of age-related memory loss, senility, dementia, Alzheimer's Disease, or a combination thereof due to a family history of one or more of these diseases or disorders. In some aspects, the subject selected for screening is suspected of having age-related memory loss, senility, dementia, or Alzheimer's Disease due to the subject's advanced age and complaints of cognitive decline or dysfunction from the subject or the subject's caretaker.

In one aspect, the sample is a blood sample.

In one aspect, the expression level of one or more biomarkers selected from IL-6, IL-10, CRP, TNFα, FABP, and PPY are measured in step (b). In another aspect, the expression level of one or more biomarkers selected from IL-6, IL-10, CRP, TNFα, FABP and PPY, and optionally the expression level of one or more of IL-7, IL-5, TNC, ICAM1, Factor VII, I309, TNFR1, A2M, TARC, eotaxin3, VCAM1, TPO, IL-18, B2M, SAA, DJ1, Aβ, tau, α-synuclein, GLP-1, PYY, insulin, HbA1c, glucose, triglycerides, HDL, LDL, vLDL, DGAT1, PPAR-γ, PPARα, and cholesterol, are measured in step (b). In one aspect, the expression level of each of IL-6, IL-10, CRP, TNFα, FABP, and PPY are measured in step (b). In another aspect, the expression level of each of IL-6, IL-10, CRP, TNFα, FABP and PPY, and optionally the expression level of one or more of IL-7, IL-5, TNC, ICAM1, Factor VII, I309, TNFR1, A2M, TARC, eotaxin3, VCAM1, TPO, IL-18, B2M, SAA, DJ1, Aβ, tau, α-synuclein, GLP-1, PYY, insulin, HbA1c, glucose, triglycerides, HDL, LDL, vLDL, DGAT1, PPAR-γ, PPARα, and cholesterol, are measured in step (b).

In some aspects, the subject is determined to have both a proinflammatory endophenotype profile and a metabolic endophenotype profile by comparing the expression level of the one or more biomarkers in the blood, plasma, or serum sample obtained from the subject to the average expression level of the one or more corresponding biomarkers from a statistical sample representative of the subject. In some aspects, the statistical sample comprises a group of individuals with Alzheimer's disease. In some aspects, the statistical sample comprises a group of individuals 50 years of age or older with Alzheimer's disease. In some aspects, the statistical sample comprises a group of individuals who do not have Alzheimer's disease.

In one aspect, the blood, plasma, or serum sample obtained from the subject is found to have a statistically different expression level of one or more biomarkers when compared to the corresponding one or more biomarkers from individuals who have been diagnosed with AD in statistical sample. In some aspects, this statistically different expression level when compared to individuals who have been diagnosed with AD in statistical sample indicates that the subject does not have a proinflammatory endophenotype profile and a metabolic endophenotype profile and the subject is not selected for inclusion in a PPAR-γ agonist clinical trial. In another aspect, the blood, plasma, or serum sample obtained from the subject is found to have a statistically similar expression level of one or more biomarkers when compared to the corresponding one or more biomarkers from individuals who have been diagnosed with AD in statistical sample. In some aspects, this statistically similar expression level when compared to individuals who have been diagnosed with AD in statistical sample indicates that the subject has a proinflammatory endophenotype profile and a metabolic endophenotype profile and the subject is selected for inclusion in a PPAR-γ agonist clinical trial. In yet another aspect, the blood, plasma, or serum sample obtained from the subject is found to have a statistically different expression level of one or more biomarkers when compared to the corresponding one or more biomarkers from individuals who do not have AD in statistical sample. In some aspects, this statistically different expression level when compared to individuals who do not have AD in statistical sample indicates that the subject has a proinflammatory endophenotype profile and a metabolic endophenotype profile and the subject is selected for inclusion in a PPAR-γ agonist clinical trial.

In one aspect, the blood, plasma, or serum sample obtained from the subject is found to have an elevated expression level of one or more biomarkers when compared to a “type” of individuals the statistical sample (e.g. when compared to either individuals diagnosed with AD or individuals who do not have AD). In another aspect, the blood, plasma, or serum sample obtained from the subject is found to have a reduced expression level of one or more biomarkers when compared to a “type” of individuals in the statistical sample. In one aspect, an elevated expression level of one or more biomarkers selected from IL-6, IL-10, CRP, and TNFα and an elevated expression level of FABP and/or PPY indicates that the subject has a proinflammatory endophenotype profile and a metabolic endophenotype profile. In some aspects, an elevated expression level of each of IL-6, IL-10, CRP, TNFα, FABP, and PPY when compared to a “type” of individuals in the statistical sample indicates that the subject has a proinflammatory endophenotype profile and a metabolic endophenotype profile. In another aspect, a reduced expression level of one or more biomarkers selected from IL-6, IL-10, CRP, and TNFα and a reduced expression level of FABP and/or PPY when compared to a “type” of individuals in the statistical sample indicates that the subject has a proinflammatory endophenotype profile and a metabolic endophenotype profile. In some aspects, a reduced expression level of each of IL-6, IL-10, CRP, TNFα, FABP, and PPY when compared to a “type” of individuals in the statistical sample indicates that the subject has a proinflammatory endophenotype profile and a metabolic endophenotype profile. In another aspect, a reduced expression level of one or more biomarkers selected from IL-6, IL-10, CRP, and TNFα and an elevated expression level of FABP and/or PPY when compared to a “type” of individuals in the statistical sample indicates that the subject has a proinflammatory endophenotype profile and a metabolic endophenotype profile. In some aspects, a reduced expression level of each of IL-6, IL-10, CRP, and TNFα and an elevated expression level of each of FABP and PPY when compared to a “type” of individuals in the statistical sample indicates that the subject has an inflammatory endophenotype profile and a metabolic endophenotype profile. In another aspect, an elevated expression level of one or more biomarkers selected from IL-6, IL-10, CRP, and TNFα and a reduced expression level of FABP and/or PPY when compared to a “type” of individuals in the statistical sample indicates that the subject has a proinflammatory endophenotype profile and a metabolic endophenotype profile. In some aspects, an elevated expression level of each of IL-6, IL-10, CRP, and TNFα and a reduced expression level of each of FABP and PPY when compared to a “type” of individuals in the statistical sample indicates that the subject has a proinflammatory endophenotype profile and a metabolic endophenotype profile. In some aspects, a subject who does not have a proinflammatory endophenotype profile and a metabolic endophenotype is not selected for inclusion in a PPAR-γ agonist clinical trial.

In one aspect, the one or more demographic factors are selected from the group consisting of age, education level, APOE ε4 allele frequency, body mass index, waist circumference, and combinations thereof. In some aspects, the age, education level, APOE ε4 allele frequency, body mass index, waist circumference, or a combination thereof is compared to the corresponding one or more demographic factors from a statistical sample representative of the subject. In some aspects, the statistical sample comprises a group of individuals with Alzheimer's disease. In some aspects, the statistical sample comprises a group of individuals 50 years of age or older with Alzheimer's disease. In some aspects, the statistical sample comprises a group of individuals who do not have Alzheimer's disease.

In one aspect, the age, education level, APOE ε4 allele frequency, body mass index, or waist circumference of the subject is statistically similar to individuals in the statistical sample who do not have Alzheimer's disease. In some aspects, this statistically similar demographic factor indicates that the subject should not undergo further screening for the PPAR-γ agonist clinical trial. In another aspect, the age, body mass index, or waist circumference of the subject is statistically lower than individuals in the statistical sample who do not have Alzheimer's disease, the education level of the subject is statistically higher than individuals in the statistical sample who do not have Alzheimer's disease, and/or the subject does not have an APOE ε4 allele, and the subject does not undergo further screening for the PPAR-γ agonist clinical trial. In another aspect, the age, education level, APOE ε4 allele frequency, body mass index, or waist circumference of the subject is statistically similar to individuals in the statistical sample who have Alzheimer's disease. In some aspects, this statistically similar demographic factor indicates that the subject should undergo further screening for the PPAR-γ agonist clinical trial. In yet another aspect, the age, body mass index, or waist circumference of the subject is statistically higher than individuals in the statistical sample who have Alzheimer's disease, the education level of the subject is statistically lower than individuals in the statistical sample who have Alzheimer's disease, and/or the subject has one or more APOE ε4 alleles, and the subject undergoes further screening for the PPAR-γ agonist clinical trial.

In one aspect, the one or more neurocognitive evaluations are selected from the group consisting of a 4-point clock drawing test, a verbal fluency test, a trail making test, a list learning test, a mini-mental state exam, and combinations thereof. In some aspects, the results of the one or more neurocognitive evaluations of the subject are compared to the corresponding one or more neurocognitive evaluations from a statistical sample representative of the subject. In some aspects, the statistical sample comprises a group of individuals with Alzheimer's disease. In some aspects, the statistical sample comprises a group of individuals 50 years of age or older with Alzheimer's disease. In some aspects, the statistical sample comprises a group of individuals who do not have Alzheimer's disease. In one aspect, the results of the one or more neurocognitive evaluations of the subject are statistically similar to individuals in the statistical sample who have Alzheimer's disease. In one aspect, this statistically similar neurocognitive evaluation result indicates that the subject should undergo further screening for the PPAR-γ agonist clinical trial. In one aspect, the results of the one or more neurocognitive evaluations of the subject are statistically different from individuals in the statistical sample who do not have Alzheimer's disease. In one aspect, this statistically different neurocognitive evaluation result indicates that the subject should undergo further screening for the PPAR-γ agonist clinical trial. In yet another aspect, the results of the one or more neurocognitive evaluations of the subject are statistically different from individuals in the statistical sample who have Alzheimer's disease. In one aspect, this statistically different neurocognitive evaluation result indicates that the subject should not undergo further screening for the PPAR-γ agonist clinical trial. In yet another aspect, the results of the one or more neurocognitive evaluations of the subject are statistically similar to individuals in the statistical sample who do not have Alzheimer's disease. In one aspect, this statistically different neurocognitive evaluation result indicates that the subject should not undergo further screening for the PPAR-γ agonist clinical trial.

In one aspect, the PPAR-γ agonist is selected from rosiglitazone and rosiglitazone XR.

### Method of Determining a Surrogate Outcome of a PPAR-γ Agonist Clinical Trial

In yet another embodiment, the present disclosure relates to a method of determining a surrogate outcome of a PPAR-γ agonist clinical trial to improve cognition or to prevent cognitive decline or dysfunction, the method comprising: (a) selecting a subject to participate in the clinical trial, wherein the subject is suspected of having age-related memory loss, senility, dementia, or Alzheimer's Disease, or having an elevated risk of age-related memory loss, senility, dementia, or Alzheimer's Disease; (b) measuring, in a blood, plasma, or serum sample obtained from the subject, the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol; (c) determining that the subject has both a proinflammatory endophenotype profile and a metabolic endophenotype profile based on the expression level of the one or more biomarkers; (d) administering a PPAR-γ agonist to a first subset of subjects and a placebo to a second subset of subjects; (e) repeating step (b) after the administration of the PPAR-γ agonist or the placebo; and (f) determining if a statistically significant change in the expression level of the one or more biomarkers is seen in the first subset of subjects when compared to any change occurring in the second subset of subjects, wherein a statistically significant change in the expression level is associated with a beneficial long term clinical outcome.

In one aspect, the subject selected for the clinical trial has an elevated risk of age-related memory loss, senility, dementia, Alzheimer's Disease, or a combination thereof due to a family history of one or more of these diseases or disorders. In one aspect, the subject selected for the clinical trial is an elderly subject. In some aspects, the subject selected for the clinical trial is suspected of having age-related memory loss, senility, dementia, or Alzheimer's Disease due to the subject's advanced age and complaints of cognitive decline or dysfunction from the subject or the subject's caretaker. In another aspect, the subject selected for the clinical trial is suspected of having age-related memory loss, senility, dementia, or Alzheimer's Disease due to the results of one or more neurocognitive evaluations. Exemplary neurocognitive evaluations are described elsewhere herein.

In one aspect, the sample is a blood sample.

In one aspect, the expression level of one or more biomarkers selected from IL-6, IL-10, CRP, TNFα, FABP, and PPY are measured in steps (b) and (e). In another aspect, the expression level of one or more biomarkers selected from IL-6, IL-10, CRP, TNFα, FABP and PPY, and optionally the expression level of one or more of IL-7, IL-5, TNC, ICAM1, Factor VII, I309, TNFR1, A2M, TARC, eotaxin3, VCAM1, TPO, IL-18, B2M, SAA, DJ1, Aβ, tau, α-synuclein, GLP-1, PYY, insulin, HbA1c, glucose, triglycerides, HDL, LDL, vLDL, DGAT1, PPAR-γ, PPARα, and cholesterol, are measured in steps (b) and (e). In one aspect, the expression level of each of IL-6, IL-10, CRP, TNFα, FABP, and PPY are measured in steps (b) and (e). In another aspect, the expression level of each of IL-6, IL-10, CRP, TNFα, FABP and PPY, and optionally the expression level of one or more of IL-7, IL-5, TNC, ICAM1, Factor VII, I309, TNFR1, A2M, TARC, eotaxin3, VCAM1, TPO, IL-18, B2M, SAA, DJ1, Aβ, tau, α-synuclein, GLP-1, PYY, insulin, HbA1c, glucose, triglycerides, HDL, LDL, vLDL, DGAT1, PPAR-γ, PPARα, and cholesterol, are measured in steps (b) and (e).

In some aspects, a statistically significant reduction in the expression level of one or more of the biomarkers measured in step (e) compared to the expression level of the corresponding biomarker in step (b) indicates that the PPAR-γ agonist is useful in improving cognition or preventing cognitive decline or dysfunction and is associated with a beneficial long term clinical outcome. In other aspects, a statistically significant increase in the expression level of one or more of the biomarkers measured in step (e) compared to the expression level of the corresponding biomarker in step (b) indicates that the PPAR-γ agonist is useful in improving cognition or preventing cognitive decline or dysfunction and is associated with a beneficial long term clinical outcome. In some aspects, step (e) occurs about one or more months after step (c). In one aspect, step (d) occurs about six months after step (d). In another aspect, step (e) occurs about twelve months after step (d). In one aspect, a statistically significant change in the expression level in step (e) at about twelve months after (d) is associated with a beneficial clinical outcome in about 24 months, about 36 months, about 48 months, or about 60 months. Therefore, in one aspect, a statistically significant change in the expression level in step (e) at about twelve months after (d) provides a surrogate outcome of the clinical trial at about 24 months, about 36 months, about 48 months, or about 60 months.

In one aspect, the PPAR-γ agonist is administered to the first subset of subjects during the clinical trial in an amount believed to be sufficient to determine if the PPAR-γ agonist is useful to improve cognition or reduce cognitive decline or dysfunction in the subject. In some aspects, the PPAR-γ agonist is administered to the first subset of subjects at a dosage of between about 0.5-20 mg, about 0.5-18 mg, about 0.5-16 mg, about 0.5-14 mg, about 0.5-12 mg, about 1.0-12 mg, about 2-10 mg, between once and eight times a day. In one aspect, the PPAR-γ agonist is selected from rosiglitazone and rosiglitazone XR. In one aspect, about 2 mg, about 4 mg, or about 8 mg of rosiglitazone or rosiglitazone XR is administered to the first subset of subjects once a day.

In one aspect, the biomarker measurements in steps (b) and (e) are obtained from an immunoassay, an enzymatic activity assay, fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In one aspect, the biomarker measurements in steps (b) and (e) are obtained by electrochemiluminescence detection.

In one aspect, the method further comprises the steps of obtaining the results of the one or more neurocognitive evaluations from the first subset and second subset of subjects before step (d), obtaining the results of the corresponding one or more neurocognitive evaluations from the first subset and second subset of subjects after step (d), and determining if the first subset of subjects demonstrated a statistically significant improvement in the one or more neurocognitive evaluations when compared to any improvement occurring in the second subset of subjects, wherein a statistically significant improvement in the one or more neurocognitive evaluations and a statistically significant change in the expression level of one or more biomarkers is associated with a beneficial long term clinical outcome.

In one aspect, the method further comprises step (g) of terminating the clinical trial when a surrogate outcome has been determined. In some aspects, a statistically significant change in the expression level of the one or more biomarkers is seen in the first subset of subjects when compared to any change occurring in the second subset of subjects indicates that a surrogate outcome of the trial has been determined. In some aspects, a statistically significant change in the expression level of the one or more biomarkers is seen in the first subset of subjects when compared to any change occurring in the second subset of subjects indicates that the clinical trial is successful in treating a subject suspected of having age-related memory loss, senility, dementia, or Alzheimer's Disease, or having an elevated risk of age-related memory loss, senility, dementia, or Alzheimer's Disease.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Proinflammatory Endophenotype

Cognitive dysfunction and decline is a major source of morbidity and mortality in the U.S. and is associated with greater health care cost, decreased treatment compliance, lost wages (patient and family), decreased productivity, poorer quality of life and gradual loss of independence. The most prominent form of cognitive loss is dementia of the Alzheimer's type; however, cognitive loss is also associated with traumatic brain injury (TBI), multiple sclerosis (MS), Parkinson's disease (PD), depression, schizophrenia, as well as many other disorders/diseases. Interestingly, inflammation is a common biological pathway that has been linked with each of these conditions as well as cognitive loss. Additionally, epidemiological studies suggest that use of anti-inflammatory medications is associated with decreased risk for cognitive loss/dementia as well as increased cognitive functioning among various disease states (e.g. TBI) though these results have been inconsistent and with many clinical trials ending in failure. To date, no prior work has been undertaken to develop a personalized medicine approach to identification of which specific patients should or should not be placed on anti-inflammatory medications in order to improve cognition. The novel method of the present invention was explicitly developed as a personalized medicine approach that identifies not only the sub-population of individuals who should be placed on anti-inflammatory medications for cognitive enhancing benefits, but equally important, which sub-population should not be placed on these medications as it is associated with greater cognitive loss. This new method can be implemented in clinical trials and practice to improve/stabilize cognition among a select sub-population of patients as well as screen out patients that should not be placed on anti-inflammatory medications due to risk of increased cognitive decline. The present inventors, have discussed the existence of a proinflammatory endophenotypes, however, this work for the first time provides a distinct endophenotypes, a combination of endophenotypes, and/or a critical therapeutic regimen as a result of the endophenotype^{3,4,15,16} In order to determine if the proinflammatory endophenotype predicted treatment response, baseline plasma samples were analyses from a previously conducted trial of the Alzheimer's Disease Cooperative Study (ADCS, Aisen et al 2003, JAMA).

Baseline plasma samples were assayed using enhanced chemiluminescence (ECL) for a range of inflammatory markers. The pro-inflammatory profile was generated using CRP and TNFα. Additional markers can be used to improve the already robust results shown herein. The frequency of the low, middle (referent group) and high ends of the pro-inflammatory profile are presented below.

TABLE 1

| | | | Fre- | Per- | Valid | Cumulative |
|---|---|---|---|---|---|---|
| Arm2 | | | quency | cent | Percent | Percent |
| placebo | Valid | Low | 6 | 8.3 | 11.1 | 11.1 |
| | | Middle | 43 | 59.7 | 79.6 | 90.7 |
| | | High | 5 | 6.9 | 9.3 | 100.0 |
| | | Total | 54 | 75.0 | 100.0 | |
| | Missing System | | 18 | 25.0 | | |
| | Total | | 72 | 100.0 | | |
| treatment | Valid | Low | 7 | 9.1 | 11.1 | 11.1 |
| | | Middle | 46 | 59.7 | 73.0 | 84.1 |
| | | High | 10 | 13.0 | 15.9 | 100.0 |
| | | Total | 63 | 81.8 | 100.0 | |

| | | |
|---|---|---|
| Missing System | 14 | 18.2 |
| Total | 77 | 100.0 |

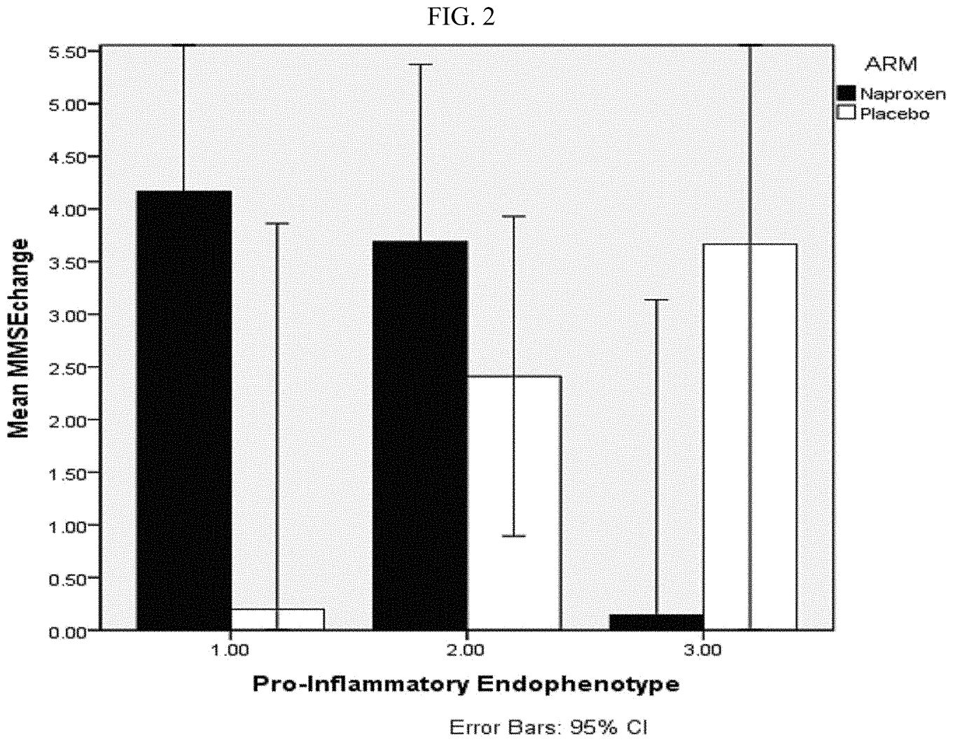

When looking at change in MMSE scores over the 12 month period of the trial, the findings were as follows: Placebo group—(a) those in the low end of the pro-inflammatory profile were stable over 12 months (stable in disease severity and cognitive functioning) when compared to the high end and the referent group (i.e. middle group), (b) those in the high end declined significantly over 12 months when compared to the referent group and the low end of the pro-inflammatory profile. Treatment group—(a) those in the low end of the pro-inflammatory profile (group 1 in FIG. 2) who were treated with an anti-inflammatory drug declined significantly faster (i.e. disease severity and cognition) when compared to the referent group (i.e. middle group; group 2 in FIG. 2)), (b) those in the high end (group 3 in FIG. 2) were stable over 12 months when treated with an anti-inflammatory drug when compared to the low end of the pro-inflammatory profile and the referent group. Therefore, treatment is indicated among those in the high end of the proinflammatory endophenotypes, but contraindicated among those in the low end of the proinflammatory endophenotype.

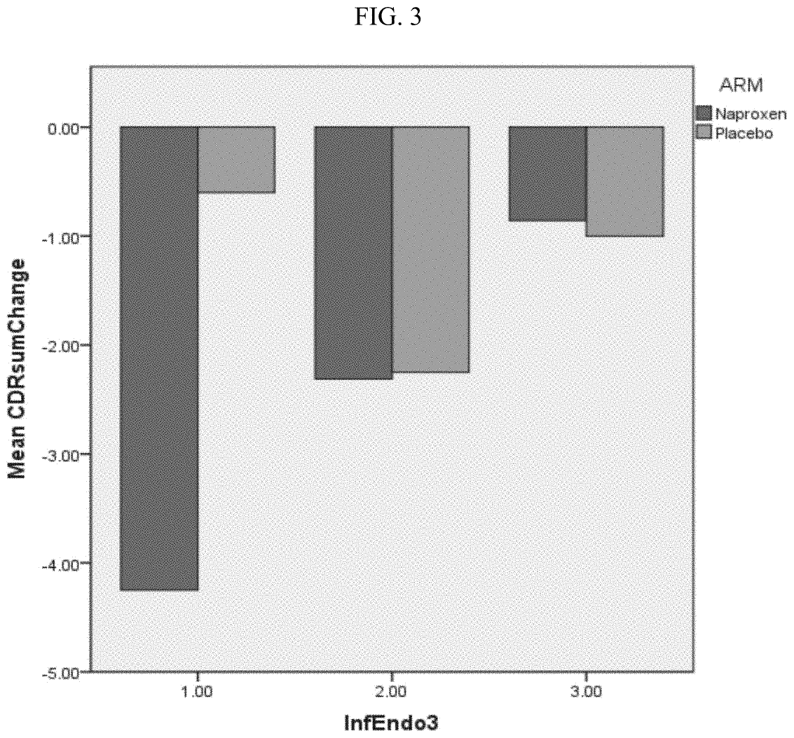

When considering disease severity (i.e., clinical dementia rating (CDR) Sum of Boxes [CDRSum]), the same was found. See FIG. 3. Specifically, those in the low end of the pro-inflammatory profile who were treated with an anti-inflammatory drug progressed in disease severity more rapidly over 12 months than any other group whereas those who were in that same biomarker-defined group declined minimally over 12 months if left untreated. On the other hand, those in the high end who were treated declined less than those who were untreated though the magnitude of difference is less than that observed from the objective cognitive measure above (i.e., mini-mental state examination (MMSE) scores).

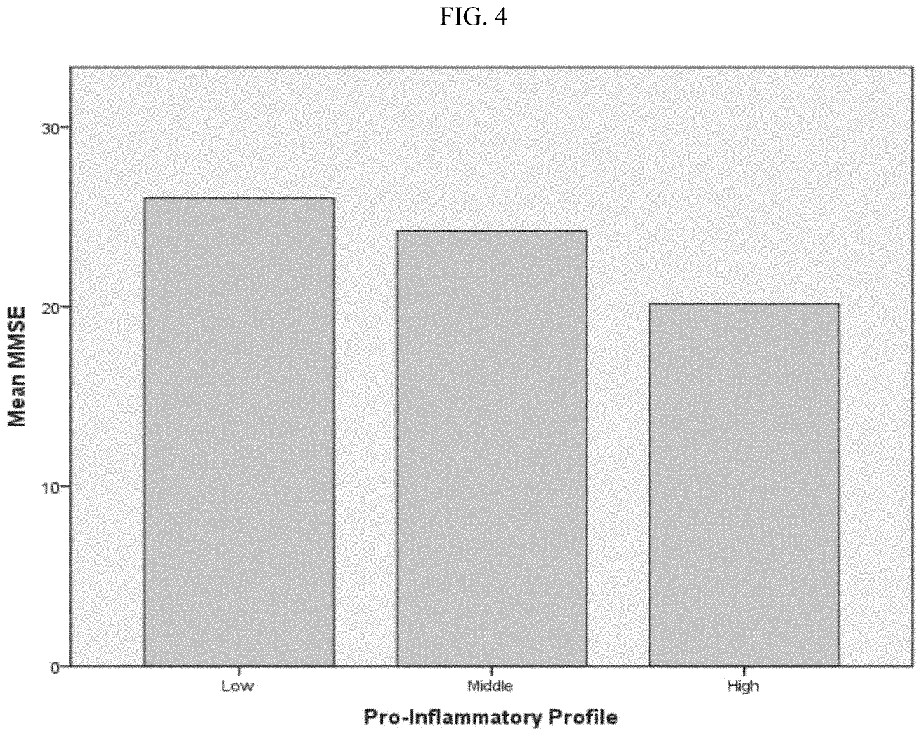

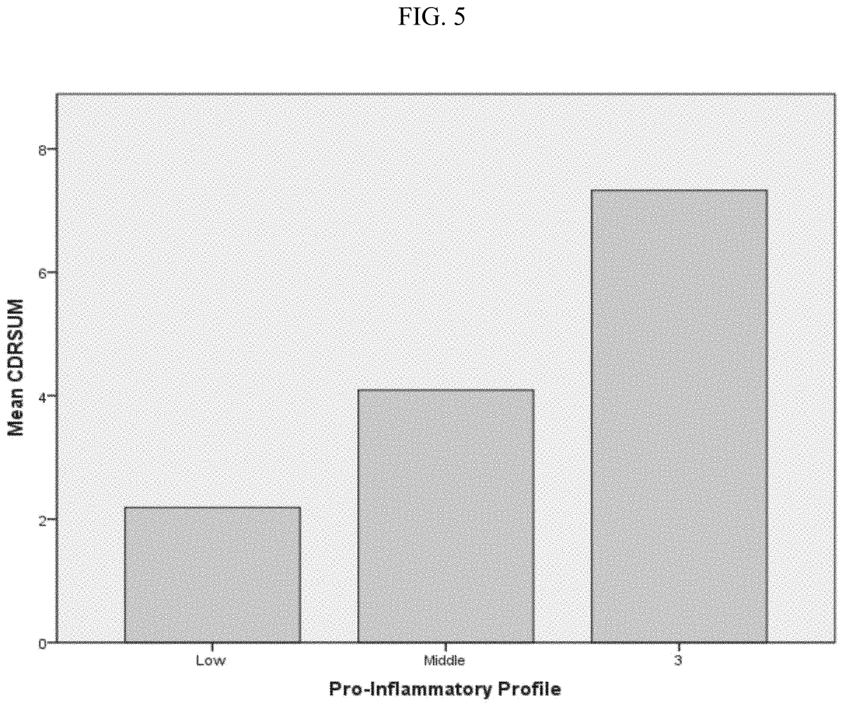

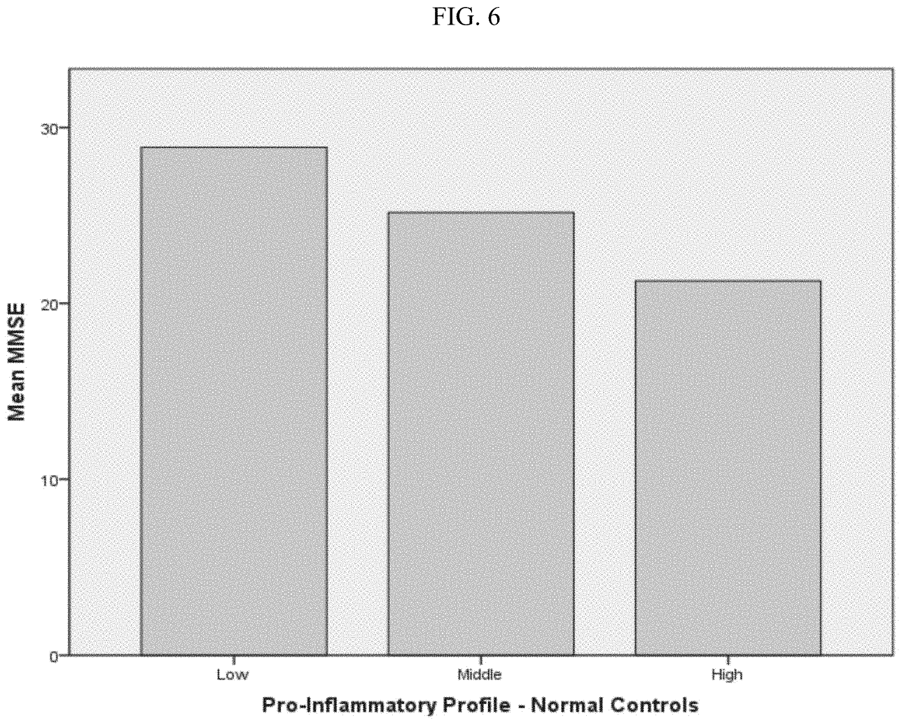

When examining baseline cognitive and disease severity markers from an independent cohort of AD cases and normal controls, the pro-inflammatory endophenotypes profile of the present invention clearly discriminated between patients' baseline characteristics. FIG. 4 shows a linear decrease in cognitive functioning (MMSE scores). FIG. 5 clearly demonstrates a linear increase in disease severity based on the pro-inflammatory profile among AD patients. FIG. 6 demonstrates the same linear decline in baseline cognitive ability (MMSE scores) among non-demented normal controls as a function of the pro-inflammatory profile.

### Example 2: Metabolic Endophenotype

As discussed herein above, cognitive dysfunction and decline is a major source of morbidity and mortality in the U.S. Interestingly, metabolic dysfunction and diabetes is a common biological pathway that has been linked with each of these conditions as well as cognitive loss. Additionally, epidemiological studies suggest that midlife diabetes is a powerful risk factor for late-life cognitive loss and that diabetes is associated with increased neuropathological burden at autopsy. As a result of this literature, several clinical trials have been conducted using diabetes and metabolic medications to treat Alzheimer's disease, Mild Cognitive Impairment (MCI) (MCI; pre-AD) with some success and several ongoing studies. In fact, one group has begun a phase 3 trial of intranasal insulin as a therapy for MCI and early AD. While there has been some success, the therapeutic benefits have been modest and no prior work has been conducted to identify the specific patients with diabetes at greatest risk for cognitive loss. The novel method of the present invention was expressly developed as a companion diagnostic method (and personalized medicine approach) that identifies the sub-population of individuals who should be placed on diabetes/metabolic medications for cognitive enhancing benefits. The present invention can be implemented in clinical trials to best select patients most likely to benefit from the treatment thereby substantially reducing the sample sizes required.

The present inventors proposed a metabolic endophenotype among MCI and AD based on (1) prior work linking diabetes and metabolic disturbance to MCI and AD and (2) and the inventors' prior biomarker and clinical work among Mexican Americans¹⁻⁴. The inventors also sought to characterize the metabolic endophenotype (MetEndo) among those diagnosed with MCI, AD and cognitively normal elders. Utilizing a multi-marker approach the present inventors have generated a metabolic endophenotypes (MetEndo). Those in the low end of the MetEndo (group 1) have minimal metabolic disturbance from a profile approach whereas those in the high end (group 3) have high levels of metabolic disturbance with all others remaining within the middle range (group 2). The inventors have found that the MetEndo predicts cognitive function and decline as well as risk for progression among those with metabolic dysfunction. The MetEndo should only be relevant for a subset of patients diagnosed with MCI and AD as the underlying neuropathology for AD is quite complex and there likely exists numerous endophenotypes. As disclosed herein the present inventors further demonstrated the existence and use of several endophenotypes including an inflammatory endophenotype^{5,6} as well as a neurotrophic factor endophenotype^{7,8} and direct methods of treatment accordingly. In fact, the present invention can even be used to retrospectively analyze blood samples from previously conducted clinical trials to demonstrate that this approach (i.e., proinflammatory endophenotype) identifies a subgroup of AD patients that benefited significantly from a previously “failed” clinical trial. Therefore, the metabolic endophenotype can be utilized to treat specific subpopulations of AD patients to slow disease progression, reduce progression from MCI to AD in select subpopulations and even prevent cognitive loss among specific subpopulations of cognitive normal elders suffering from diabetes.

When examining prevalence of the MetEndo, the inventors found that 20% of MCI patients fit into the high end of the MetEndo as compared to 5% in the low end of the MetEndo. The rate increased to 25% in the high end when restricted to MCI cases diagnosed with diabetes. Those in the low end experienced greater cognitive dysfunction and increased disease severity at baseline (Table 2) and their pathology is likely driven largely by non-metabolic factors, namely AP (see table above; number 1=low MetEndo, 2=middle group; 3=high MetEndo). Of note, the MetEndo grouping is entirely independent of clinical characterization, but all patients were diagnosed with MCI. Interestingly, when examining cognitively normal elders (NC), there was also a significant difference in cognitive outcomes by the MetEndo groupings (see Table 3). Within the NC group, the high end of the MetEndo performed most poorly with regards to cognitive outcome variables. Therefore, there is a shift in cognitive ability from NC to MCI to AD (looked the same as MCI) as a function of MetEndo.

TABLE 2
Difference in cognitive outcomes by the MetEndo groupings

| | | | N | Mean | STD |
|---|---|---|---|---|---|
| MCl | CDRSUM | 1.00 | 3 | 1.17 | 1.155 |
| | | 2.00 | 45 | 1.10 | .802 |
| | | 3.00 | 14 | .75 | .325 |
| | | Total | 62 | 1.02 | .743 |
| | SS_Combined_LM_I | 1.00 | 3 | 4.67 | 3.215 |
| | | 2.00 | 44 | 8.39 | 3.301 |
| | | 3.00 | 13 | 10.00 | 2.708 |
| | | Total | 60 | 8.55 | 3.321 |
| | SS_Combined_LM_II | 1.00 | 3 | 7.00 | 3.606 |
| | | 2.00 | 44 | 8.86 | 3.481 |
| | | 3.00 | 13 | 10.77 | 3.032 |
| | | Total | 60 | 9.18 | 3.467 |

Based on this change, the high end of the MetEndo was used to show a significant association with the progression from NC to MCI and to AD. Over a 24 month follow-up period, the highest overall rate of overall progression was found among the high end of the MetEndo group (25%) as compared to 10% among the low MetEndo group and 20% in the middle group (progression in low and middle group likely due to non-metabolic factors). Additionally, 18% of NCs in the high end of the MetEndo converted to MCI as compared to 7% of those in the low MetEndo group. A total of 34% of the MCI cases in the high MetEndo group progressed to AD within 24 months. The MetEndo was a significant predictor of progression from NC to MCI (AUC=0.63) and MCI to AD (AUC=0.60). Interestingly, 42% of the low end of the MetEndo progressed to AD over 24 months. This is likely due to the fact that (1) baseline cognition was lower in this group, and (2) the underlying pathology is loaded heavily to AP and these patients would benefit best from therapeutic agents targeting that mechanism specifically. Therefore, this method can be used for screening into the large-scale AP prevention trials (e.g. an A4 trial).

TABLE 3

| NC | | N | Mean | STD |
|---|---|---|---|---|
| CDRSUM | 1.00 | 9 | .06 | .167 |
| | 2.00 | 104 | .03 | .250 |
| | 3.00 | 32 | .11 | .535 |
| | Total | 145 | .05 | .330 |
| SS_Combined_LM_I | 1.00 | 9 | 10.33 | 3.905 |
| | 2.00 | 103 | 10.17 | 3.784 |
| | 3.00 | 31 | 9.29 | 3.466 |
| | Total | 143 | 9.99 | 3.718 |
| SS_Combined_LM_II | 1.00 | 9 | 11.22 | 2.587 |
| | 2.00 | 103 | 11.45 | 3.165 |
| | 3.00 | 31 | 10.26 | 3.109 |
| | Total | 143 | 11.17 | 3.138 |

### Example 3: Neurotrophic Endophenotype

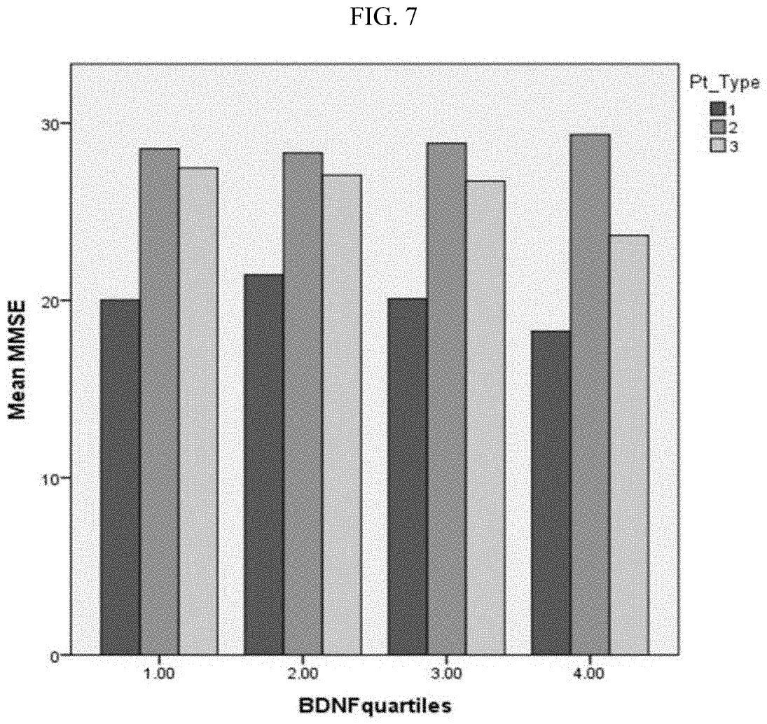

A neurotrophic endophenotype was used to evaluate and treat cognitive loss/Alzheimer's disease (AD). The inventors have shown that neurotrophic factors, such as brain derived neurotrophic factor (BDNF) could potentially be a biomarker of Alzheimer's disease presence. However, it was found that BDNF levels were not significant predictors of disease status. On the other hand, BDNF levels were significantly related to memory performance among those diagnosed with AD. It is shown herein that neurotrophic factors (i.e., BDNF, NGF, TN-3, CNTF, GDNF, LIF, and GGF) can be used to identify a subset of individuals at risk for cognitive loss specifically related to this biological system. As such, knowledge of where someone falls within this specific endophenotype will guide a specific therapy for preventing and/or treating cognitive loss. It is shown herein that by simply using BDNF levels, one can clearly demonstrate different cognitive abilities among those with and without cognitive dysfunction. In FIG. 7, it is evident that global cognitive ability (MMSE scores) varies as a function of BDNF levels by patient type (1=Alzheimer's disease, 2=normal control, 3=mild cognitive impairment).

Additionally, when examined across cognitive test scores, a clear pattern emerged. Table 4 shows that, specifically, among those with cognitive loss (AD or MCI) higher score on the neurotrophic endophenotype (range 1-4 with 4 being high levels) are associated with poorer cognitive scores and more advanced disease severity among those with cognitive loss (MCI and AD). On the other hand, higher neurotrophic endophenotype score (i.e. 4) is associated with better cognitive functioning among those who are cognitively normal. This shows that there is a shift in the importance of neurotrophic factors as an elder transitions from normal elder to cognitively impaired. By way of explanation, but in no way a limitation of the present invention, this paradoxical finding of higher BDNF levels being associated with poorer memory abilities may be due to a compensatory effect³. That is, the brain is producing higher levels of neurotrophic factors in an effort to compensate for accumulating neuropathology. In fact, this is similar to the findings and hypothesis that led to the eventual FDA approval of several cholinesterase inhibitors for the treatment of AD.

TABLE 4
Neurotrophic endophenotypes.

| PtTypeDesc | | N | Mean | Std. Deviation |
|---|---|---|---|---|
| | 1.00 | 47 | 20.02 | 5.435 |
| | 2.00 | 43 | 21.44 | 4.295 |
| MMSE | 3.00 | 74 | 20.09 | 5.626 |
| | 4.00 | 118 | 18.25 | 6.384 |
| | Total | 282 | 19.52 | 5.844 |
| AD | 1.00 | 46 | 5.33 | 3.453 |
| | 2.00 | 43 | 6.59 | 3.497 |
| CDRSUM | 3.00 | 76 | 7.29 | 4.247 |
| | 4.00 | 117 | 8.39 | 4.347 |
| | Total | 282 | 7.32 | 4.190 |
| SS_Cowat | 1.00 | 40 | 6.63 | 3.712 |
| | 2.00 | 40 | 6.53 | 2.736 |
| | 3.00 | 69 | 6.99 | 3.127 |
| | 4.00 | 99 | 6.71 | 3.444 |
| | Total | 248 | 6.74 | 3.284 |
| | 1.00 | 32 | 3.94 | 2.199 |
| | 2.00 | 35 | 3.74 | 2.501 |
| SS_Combined_LM_ I | 3.00 | 57 | 4.23 | 2.521 |
| | 4.00 | 91 | 3.47 | 2.218 |
| | Total | 215 | 3.79 | 2.350 |
| | 1.00 | 32 | 4.50 | 2.627 |
| | 2.00 | 35 | 3.57 | 1.720 |
| SS_Combined_LM_II | 3.00 | 57 | 3.91 | 1.994 |
| | 4.00 | 89 | 3.30 | 1.774 |
| | Total | 213 | 3.69 | 2.004 |
| | 1.00 | 28 | 4.50 | 2.365 |
| | 2.00 | 28 | 5.00 | 3.151 |
| SS_Combined_VR_I | 3.00 | 54 | 5.20 | 2.757 |
| | 4.00 | 98 | 4.08 | 2.903 |
| | Total | 208 | 4.55 | 2.857 |
| | 1.00 | 28 | 5.61 | 2.217 |
| | 2.00 | 28 | 4.86 | 2.068 |
| SS_Combined_VR_II | 3.00 | 54 | 4.63 | 2.095 |
| | 4.00 | 96 | 4.79 | 2.419 |
| | Total | 206 | 4.87 | 2.269 |
| | 1.00 | 102 | 27.46 | 2.349 |
| MCI MMSE | 2.00 | 64 | 27.06 | 2.429 |
| | 3.00 | 59 | 26.73 | 2.658 |
| | 4.00 | 3 | 23.67 | 4.509 |
| | Total | 228 | 27.11 | 2.515 |
| | 1.00 | 102 | .92 | .572 |
| | 2.00 | 64 | 1.10 | .851 |
| CDRSUM | 3.00 | 59 | 1.58 | 1.115 |
| | 4.00 | 3 | 1.67 | 1.041 |
| | Total | 228 | 1.15 | .867 |
| | 1.00 | 102 | 8.21 | 2.963 |
| | 2.00 | 62 | 8.19 | 3.067 |
| SS_Cowat | 3.00 | 59 | 8.76 | 3.461 |
| | 4.00 | 3 | 4.33 | 2.517 |
| | Total | 226 | 8.30 | 3.148 |
| | 1.00 | 94 | 8.68 | 3.024 |
| | 2.00 | 57 | 7.95 | 3.281 |
| SS_Combined_LM_I | 3.00 | 44 | 7.14 | 3.130 |
| | 4.00 | 3 | 5.33 | 4.041 |
| | Total | 198 | 8.08 | 3.189 |
| | 1.00 | 94 | 9.00 | 3.059 |
| | 2.00 | 57 | 8.70 | 3.600 |
| SS_Combined_LM_II | 3.00 | 44 | 7.20 | 3.218 |
| | 4.00 | 3 | 3.33 | 2.082 |
| | Total | 198 | 8.43 | 3.363 |
| | 1.00 | 102 | 8.49 | 3.414 |
| | 2.00 | 64 | 8.08 | 3.204 |
| SS_Combined_VR_I | 3.00 | 59 | 8.27 | 3.741 |
| | 4.00 | 3 | 5.67 | .577 |
| | Total | 228 | 8.28 | 3.426 |
| | 1.00 | 102 | 8.93 | 2.840 |
| SS_Combined_VR_II | 2.00 | 64 | 9.17 | 2.925 |
| | 3.00 | 58 | 8.24 | 3.570 |
| | 4.00 | 3 | 4.67 | 1.155 |
| | Total | 227 | 8.77 | 3.093 |
| | 1.00 | 158 | 28.55 | 1.808 |
| | 2.00 | 136 | 28.32 | 2.427 |
| MMSE | 3.00 | 136 | 28.85 | 1.856 |
| | 4.00 | 127 | 29.35 | 1.088 |
| | Total | 557 | 28.75 | 1.898 |
| | 1.00 | 158 | .00 | .040 |
| | 2.00 | 136 | .04 | .279 |
| CDRSUM | 3.00 | 136 | .01 | .074 |
| | 4.00 | 127 | .00 | .044 |
| | Total | 557 | .01 | .146 |
| | 1.00 | 154 | 9.36 | 3.122 |
| | 2.00 | 133 | 8.52 | 3.507 |
| NC_SS_Cowat | 3.00 | 134 | 9.74 | 3.640 |
| | 4.00 | 126 | 11.40 | 3.025 |
| | Total | 547 | 9.72 | 3.473 |
| | 1.00 | 152 | 9.79 | 3.321 |
| | 2.00 | 130 | 9.78 | 3.311 |
| SS_Combined_LM_I | 3.00 | 120 | 11.38 | 3.644 |
| | 4.00 | 116 | 13.15 | 3.116 |
| | Total | 518 | 10.91 | 3.611 |
| | 1.00 | 152 | 11.11 | 3.037 |
| | 2.00 | 129 | 10.95 | 2.904 |
| SS_Combined_LM_II | 3.00 | 120 | 12.35 | 3.145 |
| | 4.00 | 116 | 13.66 | 2.690 |
| | Total | 517 | 11.93 | 3.137 |
| | 1.00 | 147 | 9.53 | 3.482 |
| | 2.00 | 119 | 8.68 | 3.687 |
| SS_Combined_VR_I | 3.00 | 118 | 10.47 | 3.858 |
| | 4.00 | 127 | 12.14 | 3.342 |
| | Total | 511 | 10.20 | 3.797 |
| | 1.00 | 147 | 11.46 | 3.048 |
| | 2.00 | 119 | 10.72 | 3.045 |
| SS_Combined_VR_II | 3.00 | 118 | 12.06 | 3.565 |
| | 4.00 | 127 | 13.28 | 3.196 |
| | Total | 511 | 11.88 | 3.332 |

### Example 4: Method for Identifying Patients for a Personalized Medicine Approach to Treating and Preventing Cognitive Loss

Cognitive dysfunction and decline is a major source of morbidity and mortality in the U.S. Cognitive dysfunction is associated with greater health care cost, decreased treatment compliance, lost wages (patient and family), decreased productivity, poorer quality of life and gradual loss of independence. The most prominent form of cognitive loss is dementia of the Alzheimer's type; however, cognitive loss is also associated with traumatic brain injury (TBI), multiple sclerosis (MS), Parkinson's disease (PD), depression, schizophrenia, as well as many other disorders/diseases. Interestingly, inflammation is a common biological pathway that has been linked with each of these conditions as well as cognitive loss. The inventors have previously generated a blood-based method for (1) identification of Alzheimer's Disease and (2) detecting and discriminating between neurodegenerative diseases. However, these data also suggest that the biological algorithms and endophenotypes generated can also distinguish cognitive ability among those within the pre-AD stage of Mild Cognitive Impairment as well as among cognitively normal adults and elders. The methods taught herein can also identify those at greatest risk for cognitive decline. A purpose of the current invention is the introduction of a method for selecting patients into trials aimed at preventing and/or treating cognitive loss based on the disclosed endophenotype methods.

The primary method for selecting patients into clinical trials is on disease diagnosis. However, most diseases have incredibly complex etiologies (e.g. diabetes, heart disease, Alzheimer's disease, depression). The approach begins with the patient presenting with a diagnosis of cognitive loss. Therefore, the current methods are directed towards the diagnosis of cognitive loss, independent of disease state. The cognitive loss may be due to any number of underlying conditions including, but not limited to Alzheimer's disease, Parkinson's disease, Multiple Sclerosis, stroke, other neurodegenerative or neurological disease, depression or other affective disturbance, diabetes and other metabolic disturbance, heart disease, and thyroid disease. Once identified as having or at risk for cognitive loss, the personalized medicine approach can also be used.

When examining baseline cognitive and disease severity markers for those diagnosed with Alzheimer's disease and normal controls, the present inventors have shown that inflammatory profiles can discriminate between cognitive abilities.

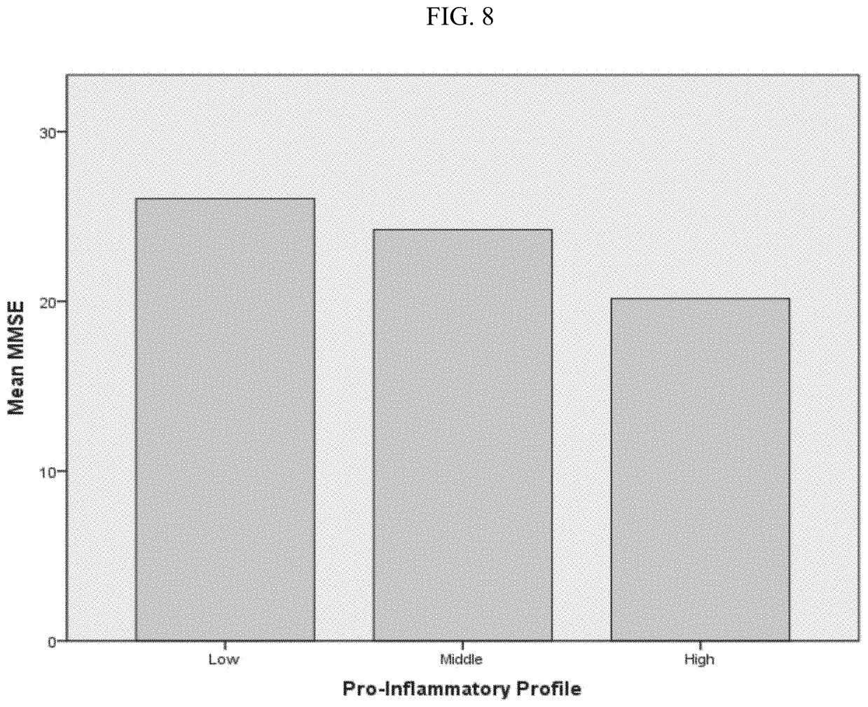

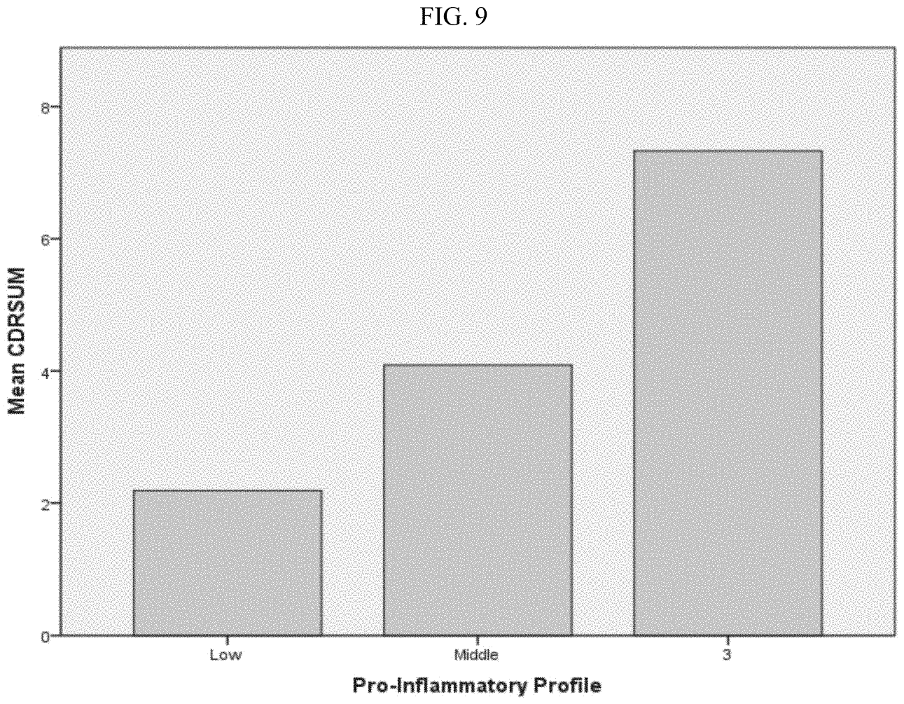

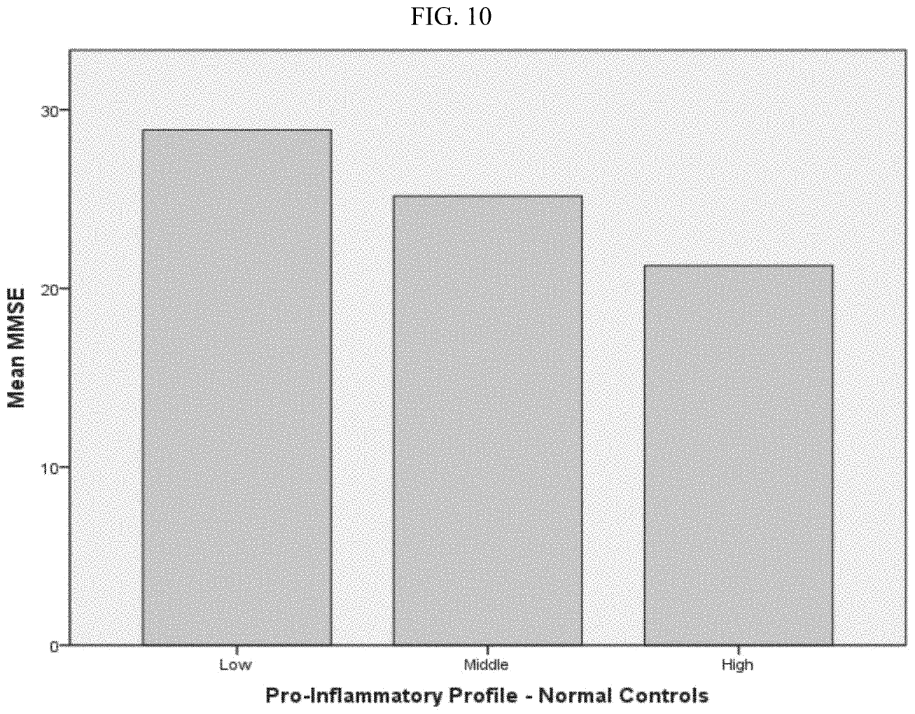

FIG. 8 shows a linear decrease in cognitive functioning (MMSE scores). When examining baseline cognitive and disease severity markers for those diagnosed with Alzheimer's disease and normal controls, the inventors show herein that inflammatory profiles can discriminate between cognitive abilities. FIG. 8 shows a linear decrease in cognitive functioning (MMSE scores). FIG. 9 demonstrates a linear increase in disease severity based on the pro-inflammatory profile among AD patients. FIG. 10 demonstrates the same linear decline in baseline cognitive ability (MMSE scores) among non-demented normal controls as a function of the pro-inflammatory profile.

### Example 5: Method for Producing Prognostic Models of Patient Responses to Therapeutic Molecules

Billions of dollars has been spent on “failed” clinical trials. A key flaw to the current design of most trials is the selection of patient populations. Specifically, patients are typically screened into trials based on a heterogeneous disease classification rather than the specific biology of the drug and the patient's baseline biological profile. As an example, Alzheimer's disease clinical trials recruit based on a clinical diagnosis of NINDS-ADRDA (or newer NIA-AA) criteria “Probable Alzheimer's Disease” without regard to any specific underlying biological mechanism linked to AD itself. Because of the “one-size-fits-all” approach to many clinical trials seeking a single cure/treatment for a complex disease process, there are thousands of previously conducted “failed” trials with potentially useful therapeutic molecules that will not make it to patients who would benefit most by those particular medications. It is also well-known that all trials have responders and non-responders, but the trials are designed to look for group-level effects rather than sub-populations.

The identification of patients most likely to be responders, non-responders and adverse responders to therapeutic agents has tremendous potential for revolutionizing medical practice. Currently, the majority of clinical trials enroll patients by heterogeneous disease categorizations (e.g. Alzheimer's disease, Multiple Sclerosis, Parkinson's disease, COPD, chronic kidney disease) rather than sub-categorizations of patients most likely to respond to a given therapy. A method for the generation of companion diagnostic tools explicitly designed to identify those patients most likely to benefit is shown herein. It has the further advantage that the present invention has no impact (negative consequences) on previously conducted clinical trials. Thus, this method (outlined briefly below) can then be used to: (1) target medications to specific patient populations and even (2) generate new clinical trials that enroll specific patients most likely to benefit from the specific drug itself.

Broadly, the methods generated for use here monitor dysfunction within multiple biological systems including inflammation, neurotrophic factors, and metabolic dysfunction. Other systems can be also be targeted to the specific therapeutic molecule as deemed appropriate for a particular candidate drug. These systems are monitored via proteomic analyses though genomic (as well as other) markers can be incorporated as needed for the particular compound. It is important to note that this is not a single-marker approach. The superiority of multi-marker approaches when considering proteomic analyses as applied to complex diseases has already been shown hereinabove. Therefore, overall dysfunction of the system is monitored rather than the method being skewed by any single marker. With appropriate sample sizes within each individual trial analyzed, the systems are monitored via advanced bioinformatics (e.g. structural equation modeling, random forest analysis, support vector machines).

Once the specific systems are selected for monitoring and samples identified, the approach can be applied in a variety of ways. However, the optimal approach is as follows, which requires multiple previously-conducted trials (e.g. Phase 2a, Phase 2b, Phase 3).

Step 1. Generation of the prediction model. This approach will take 2 forms:

(1) a priori definition of the systems and how they will predict treatment response (positive, negative and no response); (2) a theoretical discovery of the optimal prediction algorithm for detection of responders, non-responders and adverse responders. This entire step takes place in the initial clinical trial completely independent of all other trials.

Step 2. Application and refinement of the model. Once the model is generated from Step 1, it is applied blindly to the second clinical trial to predict outcomes. Next, the process in Step 1 is used again in this second trial to further refine the predictive algorithm.

Step 3. Validation of the model. Once the model has been generated and refined, it is then applied to the first Phase 3 clinical trial to determine the efficacy of the model in predicting treatment responders, non-responders and adverse responders. If a second Phase 3 trial is available, the model is applied again with further refinement if necessary.

1. The outcome variables of treatment response are open. For example, for MS the outcome can be relapse rates, but also quality of life, daily living ability, depression rates, cognitive ability, or whatever outcome of interest to the user.

2. The product at the end of the project is designed to be a companion diagnostic that can be used to (a) select patients for targeted therapy and/or (b) design a new clinical trial that specifically targets only those patients most likely to respond.

The current methodology provides a method for refining target populations to therapeutic molecules. Despite the fact that most therapeutic molecules do not make it through Phase 3 trials, many of these molecules have considerable impact for sub-populations of patients. However, a company cannot post hoc analyze a clinical trial and present that information to the FDA. On the other hand, the method taught herein provides a novel way for the identification of treatment responders, non-responders and adverse responders which can then be used to: (1) target specific patient populations with FDA approved drugs, as well as (2) design additional Phase 3 trials that will selectively enroll (and rule out) target populations to demonstrate efficacy of these therapeutic molecules.

Blood samples from a previously conducted clinical trial among AD patients was used. This trial was conducted by the Alzheimer's Disease Cooperative Study (ADCS, Aisen et al 2003, JAMA).

Baseline plasma samples were assayed using ECL for a range of inflammatory markers. The pro-inflammatory profile was generated using CRP and TNFα. The frequency of the low, middle (referent group) and high ends of the pro-inflammatory profile are presented in Table 5.

Table 5 is a summary of the changes in MMSE scores over the 12 month period of the trial.

InfEndo3

| | | | | | | Cumu- |
|---|---|---|---|---|---|---|
| | | | Fre- | Per- | Valid | lative |
| Arm2 | | | quency | cent | Percent | Percent |
| placebo | Valid | Low | 6 | 8.3 | 11.1 | 11.1 |
| | | Middle | 43 | 59.7 | 79.6 | 90.7 |
| | | High | 5 | 6.9 | 9.3 | 100.0 |
| | | Total | 54 | 75.0 | 100.0 | |
| | | Missing System | 18 | 25.0 | | |
| | | Total | 72 | 100.0 | | |
| treat- | Valid | Low | 7 | 9.1 | 11.1 | 11.1 |
| ment | | Middle | 46 | 59.7 | 73.0 | 84.1 |
| | | High | 10 | 13.0 | 15.9 | 100.0 |
| | | Total | 63 | 81.8 | 100.0 | |
| | | Missing System | 14 | 18.2 | | |
| | | Total | 77 | 100.0 | | |

Table 5 is a summary of the changes in MMSE scores over the 12 month period of the trial. The findings were as follows: Placebo group—(a) those in the low end of the pro-inflammatory profile were stable over 12 months (stable in disease severity and cognitive functioning) when compared to the high end and the referent group (i.e. middle group), (b) those in the high end declined significantly over 12 months when compared to the referent group and the low end of the pro-inflammatory profile.

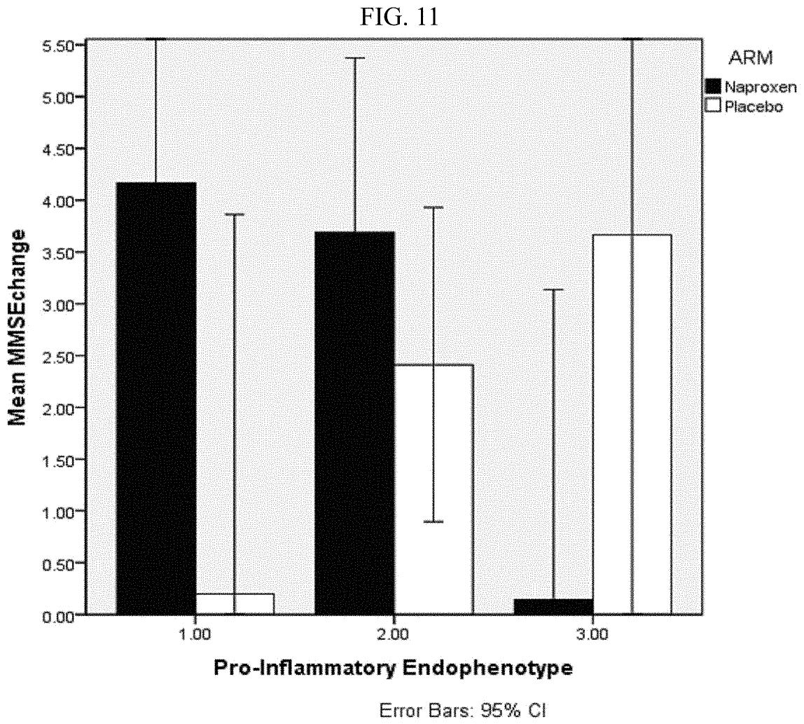

FIG. 11 shows the results for the treatment group—(a) those in the low end of the pro-inflammatory profile (group 1 in FIG. 11) who were treated with an anti-inflammatory drug declined significantly faster (i.e. disease severity and cognition) when compared to the referent group (i.e. middle group; group 2 in FIG. 11)), (b) those in the high end (group 3 in FIG. 11) were stable over 12 mo when treated with an anti-inflammatory drug when compared to the low end of the pro-inflammatory profile and the referent group.

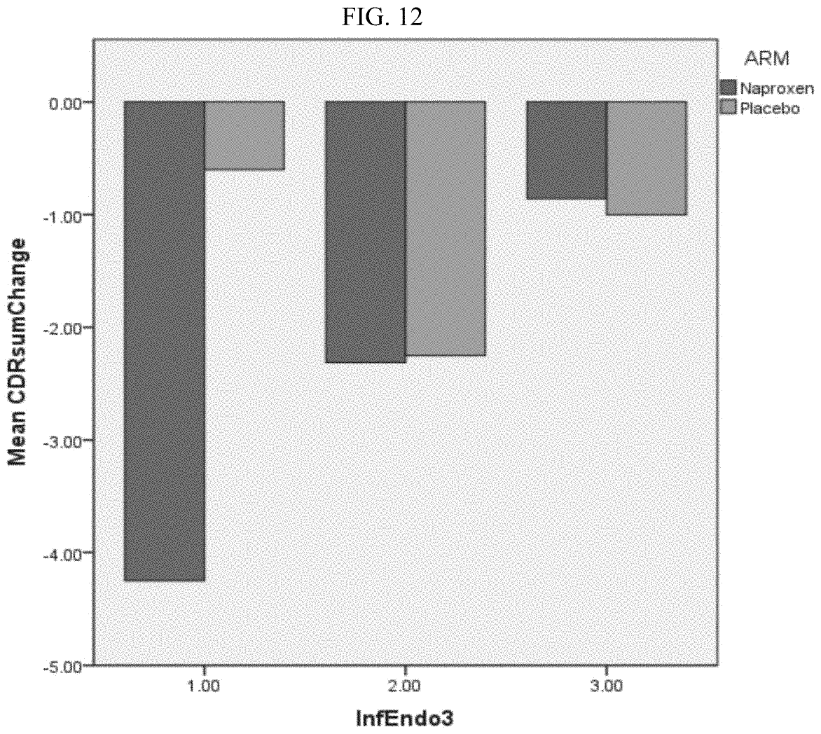

When considering disease severity (i.e. CDR Sum of Boxes [CDRSum]), the same was found. See FIG. 12. Specifically, those in the low end of the pro-inflammatory profile who were treated with an anti-inflammatory drug progressed in disease severity more rapidly over 12 mo than any other group whereas those who were in that same biomarker-defined group declined minimally over 12 months if left untreated. On the other hand, those in the high end who were treated declined less than those who were untreated though the magnitude of difference is less than that observed from the objective cognitive measure above (i.e. MMSE scores).

These data demonstrate the effectiveness of the methods and how these methods will be applied to numerous other disease states (e.g. Multiple Sclerosis).

### Example 6: A Precision Medicine Model for Prevention of Alzheimer's Disease Using NSAID Therapy: Application to the ADAPT Study

To date, Alzheimer's disease (AD) therapeutic trials (and approved therapies) utilize a “one-size-fits-all” approach assuming that each and every patient is the same and will respond to the same intervention strategies. AD has an annual health care cost similar to that of cardiovascular disease and more than cancer; however, while death rates due to cancer have declined in recent decades, death rates due to AD have steadily increased. This discrepancy is likely largely due to significantly improved treatment response in cancer therapeutics that have come as a result of the precision medicine model. AD is not a homogeneous group, but rather, there are subgroups that may respond to given therapies. Herein, the precision medicine model was tested for targeted NSAID therapy for preventing AD by leveraging samples and data from the ADAPT study (Adapt Research Group, et al., “Naproxen and celecoxib do not prevent AD in early results from a randomized controlled trial,” Neurology, 2007, 68:1800-1808).

While a precision medicine approach of targeting specific subpopulations of patients most likely to respond to a given therapy has been proposed for AD, few studies have explicitly tested the paradigm. Precision medicine is, at its core, a companion diagnostic (CDx) driven therapy, an approach has led to significant advancements in cancer therapeutics. In 2001, Spear and colleagues estimated that efficacy rates in oncology were about 25%. Subsequently, significant improvements have been seen through the use of CDx driven therapy following the development of trastuzumab for the treatment of specific patients with a particular biomarker positive form of breast cancer. Herein, a precision medicine approach was applied to provide proof-of-concept for the use of blood-based biomarkers for the context of use (COU) of CDx for preventing AD using NSAIDs.

In this study, baseline plasma samples were assayed from n=351 patients with mild-to-moderate AD who were enrolled into a 1-year multicenter, randomized, double-blind, placebo-controlled study of rofecoxib (25 mg once daily) and naproxen (220 mg twice-daily) (Aisen, P. S. et al., “Effects of rofecoxib or naproxen vs placebo on Alzheimer disease progression: a randomized controlled trial,” JAMA, 2003, 289:2819-2826). In this study, it was found that drug-specific CDx could be created using CRP, IL6, IL10 and TNFα. The CDx-naproxen was 97% accurate in predicting treatment response and the CDx-rofecoxib was 98% accurate in predicting treatment response. Herein, whether the same proteomic markers at baseline could be used to predict treatment response in the previously conducted ADAPT study was studied.

Another goal of the current study was to conduct pilot analyses on the utility of change in blood-based biomarkers over time as surrogate outcomes. According to FDA guidance, surrogate endpoints may be used instead of clinical outcomes in clinical trials. In fact, nearly 50% of new drugs approved by the FDA from 2010-2012 were based on surrogate endpoints. If a surrogate endpoint clearly predicts a beneficial effect through appropriate studies, the surrogate endpoint can facilitate more efficient drug development programs by reducing trial duration. As an example, if a surrogate biomarker changes at 6- or 12-months and change in this biomarker is associated with long-term (e.g., 36 mo or 60 mo) clinical outcomes, the trial can be designed to detect the surrogate biomarker outcome at 6- or 12-months. In already approved clinical trials, surrogate endpoints were utilized across numerous disease states (e.g. cancer, Hepatitis, HIV); however, none in neurodegenerative diseases. While it was been assumed that cerebral amyloid would serve as a surrogate biomarker outcome for trials in AD, and these anti-amyloid medications have been shown to remove cerebral amyloid, the reduction in cerebral amyloid has yet to be clearly associated with clinically meaningful long-term outcomes for those with dementia. Herein, the hypothesis that changes in proteomic markers can serve the COU of surrogate outcomes was studied. The availability of surrogate biomarkers could revolutionize trail design by substantially reducing trial duration, thereby reducing trial costs and increasing patent life. Proof-of-concept analyses were conducted by examining change in inflammatory marker levels over time in the ADAPT study.

Herein, a proof-of-concept precision medicine approach was applied using blood-based biomarker profiling for the generation of CDx-guided NSAID therapy specific for patients who are at a higher risk for AD (age and family history). In certain embodiments, randomized clinical trial using NSAIDs for treating or preventing AD should be successful for specific subgroups of patients if appropriate companion diagnostics are utilized for the identification of the specific patients who are most likely to respond as well as exclusion of patients most likely to suffer adverse responses. One aim of the current study was to test this hypothesis in one of these trials using existing blood samples and data from a previously conducted clinical trial testing the efficacy of two NSAIDs (naproxen and celeboxib) for the treatment of AD (Aisen, P. S. et al., “Neither rofecoxib nor naproxen slows cognitive decline in people with mild-to-moderate Alzheimer's disease,” Evidence-Based Healthcare, 2003, 7:200-201).

### Methods

### Participants

Participants included in this study were enrolled in the Alzheimer's Disease Anti-Inflammatory Prevention Trial (ADAPT). A full characterization of the sample has been previously published. ADAPT was a multi-site, randomized, double-blind, parallel assignment trial conducted over the span of 45 months. The aim of the ADAPT trial was to examine if the use of non-steroidal anti-inflammatory medications (Naproxen and Celecoxib) could delay or prevent the onset of AD and age-related cognitive decline. A total of n=2625 participants were recruited between March of 2001 and December of 2004 and randomized into one of the following treatment arms: Celecoxib (200 mg b.i.d 4 per day), Naproxen sodium (220 mg b.i.d. 4 per day), or placebo. A total of 145 completed (512 randomized) the placebo arm, 99 completed (343 randomized) the Celecoxib arm and 98 completed (337 randomized) the Naproxen arm.

### ADAPT Trial Inclusion and Exclusion Criteria

Inclusion Criteria. Inclusion criteria required participants be age 70 or older with a family history of age-related memory loss, senility, dementia, or Alzheimer's disease.

Exclusion Criteria. Exclusion criteria included a history of cognitive impairment or dementia; hypersensitivity to aspirin, ibuprofen, celecoxib, naproxen, or other NSAIDs; use of anti-coagulant medication; current alcohol abuse or dependence; history of peptic ulcer disease with bleeding or obstruction; clinically significant liver or kidney disease.

### Assays

Blood samples obtained from the ADAPT clinical trial were assayed at the Institute for Translational Research (ITR) biomarker core.

Sample Preparation. Samples were prepared for proteomic assay using the Hamilton Robotic StarPlus system, which is an automated liquid handling workstation that improves the quality of assays, QA/QC monitoring, and proteomic capacity in the ITR biomarker core. Re-aliquoting, when necessary, was conducted with the Hamilton easyBlood robotic system.

Sample Assay. Plasma samples were assayed on a multi-plex biomarker assay platform using electrochemiluminescence (ECL) technology, which uses labels that emit light when electrochemically stimulated thereby improving sensitivity of detection of analytes even at low concentrations. ECL is a well-established technology recognized for its benefits of increased sensitivity and requiring less volume as compared to conventional ELISAs, which is the gold standard for most assays. Plasma samples were assayed for a targeted proteomic panel based on prior work linking them with AD and included the following: fatty acid binding protein (CV=2.2; LLOD=7.4 pg/mL), beta 2 microglobulin (CV=10.1, LLOD=2.9 pg/mL), pancreatic polypeptide (CV=16.2; LLOD=66.7 pg/mL), CRP (CV=5.2; LLOD=2.1 pg/mL), ICAM-1 (CV=6.6; LLOD=1.8 pg/mL), thrombopoeitin (CV=5.9; LLOD=1.5 pg/mL), α2 macroglobulin (CV=3.6; LLOD=339.5 pg/mL), exotaxin 3 (CV=3.3; LLOD=0.7 pg/mL), tumor necrosis factor α (CV=4.2; LLOD=0.05 pg/mL), tenascin C (CV=6.6; LLOD=1.3 pg/mL), interleukin (IL)-5 (CV=4.4; LLOD=0.05 pg/mL), IL-6 (CV=7.3; LLOD=0.04 pg/mL), IL-7 (CV=1.9; LLOD=0.07 pg/mL), IL-10 (CV=4.6; LLOD=0.02 pg/mL), IL-18 (CV=2.3; LLOD=0.05 pg/mL), 1-309 (CV=8.5; LLOD=0.1 pg/mL), Factor VII (CV=8.8; LLOD=2.1 pg/mL), VCAM 1 (CV=5.4; LLOD=5.2 pg/mL), TARC (CV=3.3; LLOD=0.06 pg/mL) and SAA (CV=10.9; LLOD=10.4 pg/mL).

### Statistical Analyses

Statistical analyses were conducted in R (v3.4.2). Support vector machine (SVM) were utilized to generate predictive biomarker profiles. SVM derives decision planes that define decision boundaries and serves as a classifier method by performing classification tasks through the construction of hyperplanes in a multidimensional space thereby separating cases of different class labels. The sample was randomly split (70/30) into training and test samples with diagnostic accuracy calculated by receiver operating characteristic (ROC) curves derived from the test sample. Treatment response was defined as an MMSE score that was stable or improved over the trial duration whereas other was defined as either unknown or non-responder based on a decline in the MMSE score.

### Results

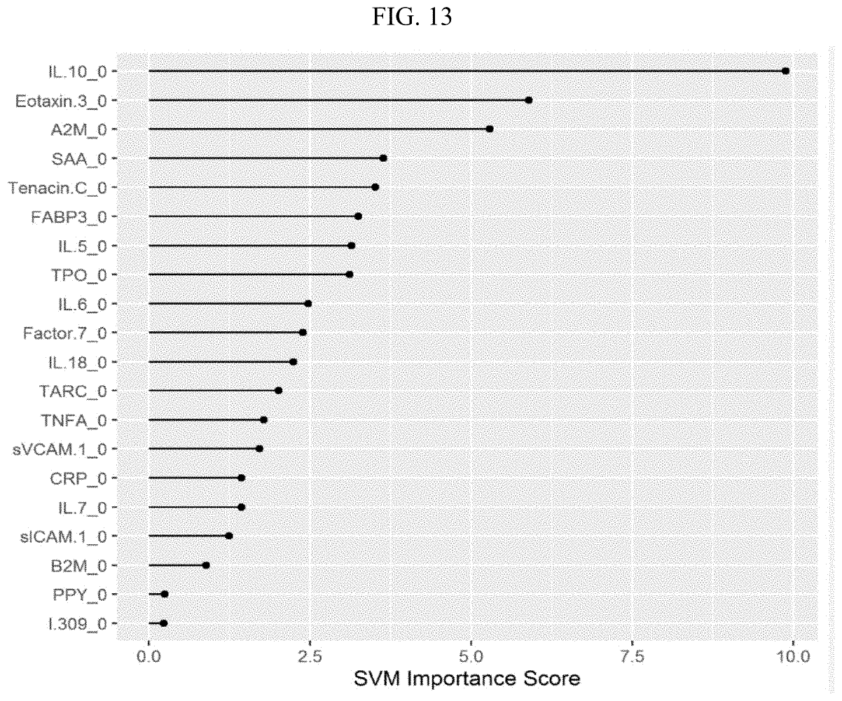

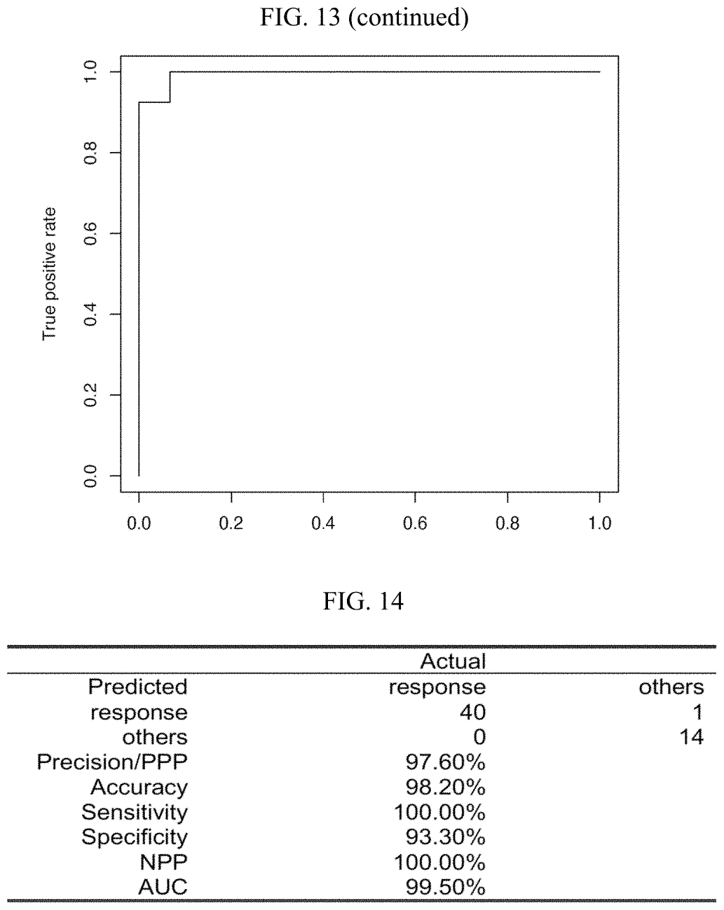

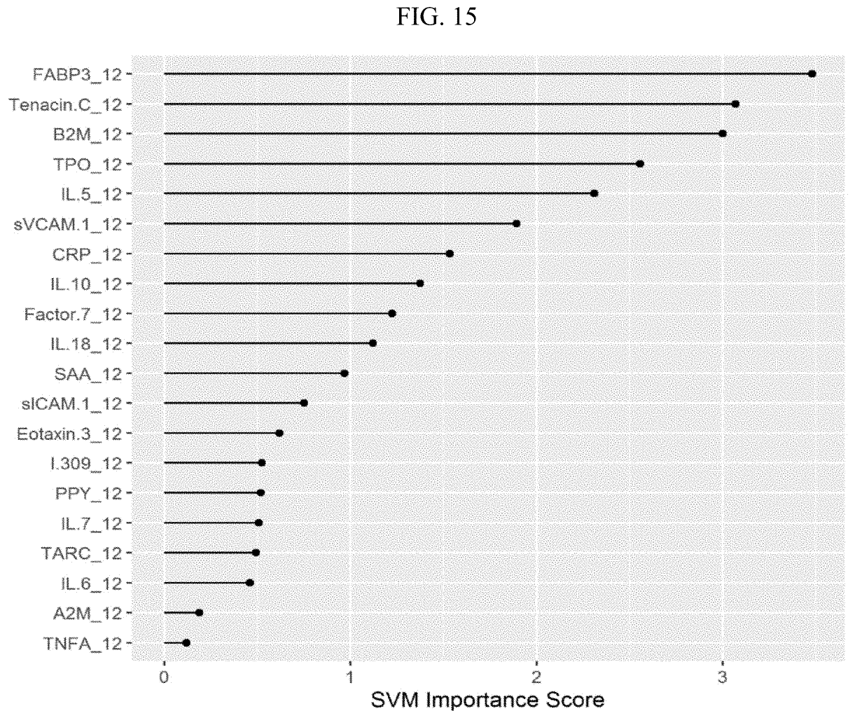

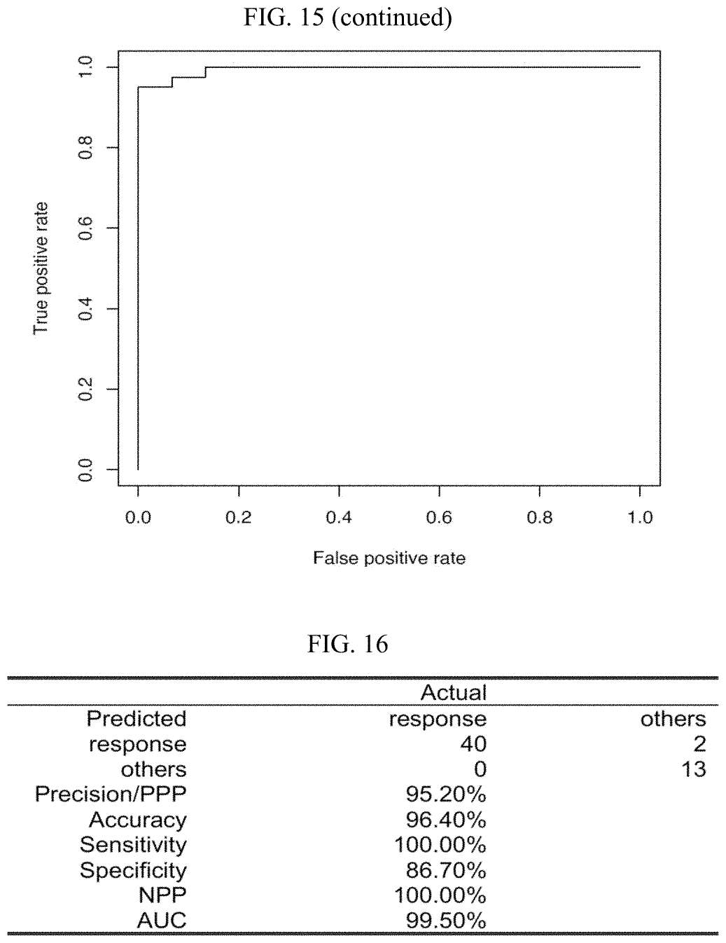

For the present study, plasma assays were derived from n=193 baseline (Celecoxib n=60, Naproxen n=51, Placebo n=82) and n=562 12-month (Celecoxib n=157, Naproxen n=159, Placebo n=246) samples across treatment arms. Table 6 provides an outline of the descriptive characteristics for the ADAPT trial participants split by treatment arm. For the Celecoxib treatment arm, there were 40 responders and 15 others (adverse responder, unknown) when measuring change in MMSE score from baseline to 12 months. The full 20-protein predictive biomarker algorithm when using baseline proteomics (with an optimal cut-off score of 0.886) reached an area under the curve (AUC) of 99.5% with a sensitivity of 1.00 and specificity of 0.93 (see FIG. 13 and FIG. 14). When utilizing proteomics at 12 months, the same 20-protein predictive biomarker algorithm reached the same AUC of 99.5% with a sensitivity of 1.00 and specificity of 0.87 (with an optimal cut-off score of 0.817) (See FIG. 15 and FIG. 16).

TABLE 6
Demographic characteristics of the ADAPT trial participants
split by study arm at baseline and 12 months

| | Total | Celecoxib | Naproxen | Placebo |
|---|---|---|---|---|
| 0 months | | | | |
| No. randomized | 193 | 60 | 51 | 82 |
| Age percentiles | | | | |
| 50 | 73.6 | 73.2 | 73 | 74.1 |
| 25, 75 | 71.7, 77.5 | 71.3, 77.1 | 71.6, 77.0 | 72.4, 78.8 |
| 0, 100 | 70.0, 86.4 | 70.1, 85.4 | 70.2, 83.9 | 70.0, 86.4 |
| Gender, % | | | | |
| Female | 40.4 | 40 | 37.3 | 42.7 |
| Male | 59.6 | 60 | 62.7 | 57.3 |
| Education, % | | | | |
| Less than high | 0 | 0 | 0 | 0 |
| school | | | | |
| High school degree | 23.8 | 16.7 | 21.6 | 30.5 |
| College, no degree | 48.7 | 55 | 49 | 43.9 |
| College degree | 27.5 | 28.3 | 29.4 | 25.6 |
| Incdem, % | | | | |
| 0 | 97.9 | 98.3 | 98 | 97.6 |
| 1 | 2.1 | 1.7 | 2 | 2.4 |
| 12 months | | | | |
| No. randomized | 562 | 157 | 159 | 246 |
| Age percentiles | | | | |
| 50 | 74.1 | 74.2 | 74.1 | 74.1 |
| 25, 75 | 71.8, 77.5 | 71.8, 77.2 | 71.8, 78.0 | 71.8, 77.7 |
| 0, 100 | 70.0, 88.8 | 70.0, 87.1 | 70.0, 87.4 | 70.0, 88.8 |
| Gender, % | | | | |
| Female | 45.7 | 45.9 | 50.9 | 42.3 |
| Male | 54.3 | 54.1 | 49.1 | 57.7 |
| Education, % | | | | |
| Less than high | 0 | 0 | 0 | 0 |
| school | | | | |
| High school degree | 24.6 | 21.7 | 23.3 | 27.2 |
| College, no degree | 47.7 | 52.2 | 46.5 | 45.5 |
| College degree | 27.8 | 26.1 | 30.2 | 27.2 |
| Incdem, % | | | | |
| 0 | 97.3 | 95.5 | 97.5 | 98.4 |
| 1 | 2.7 | 4.5 | 2.5 | 1.6 |

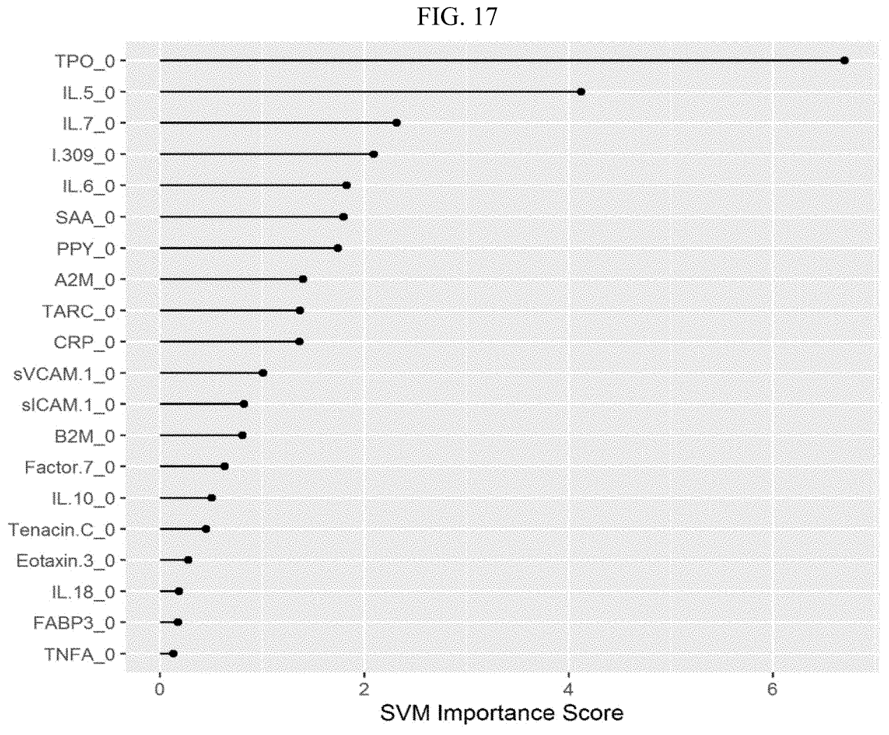

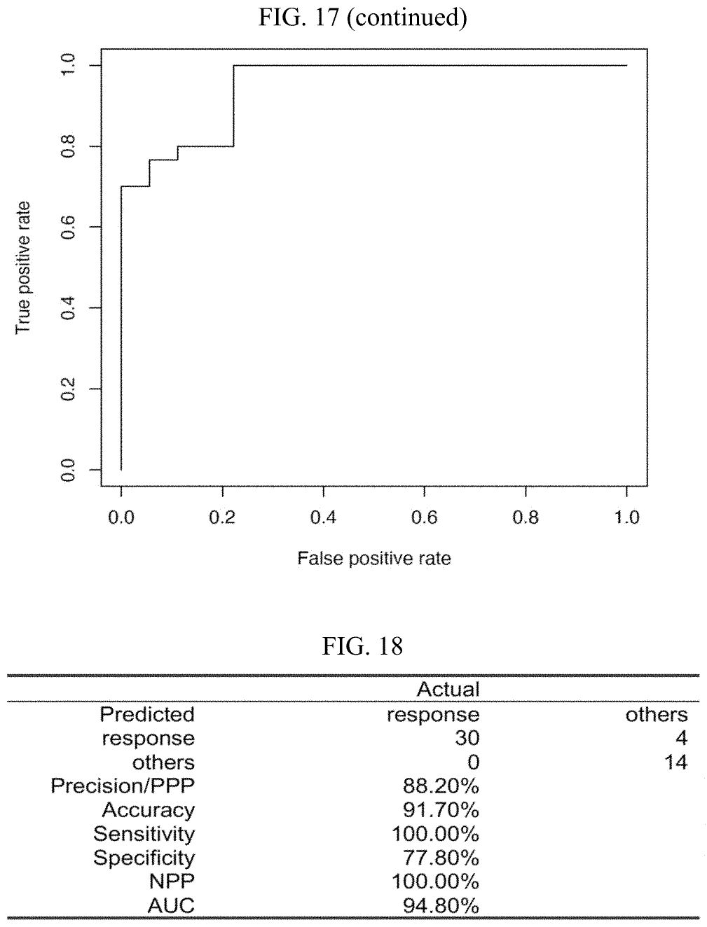

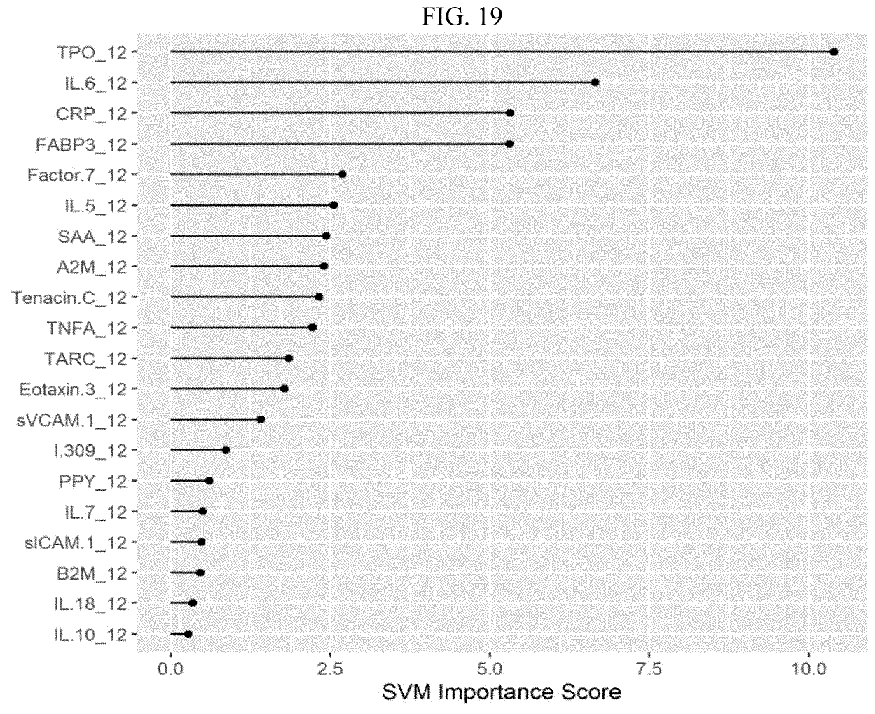

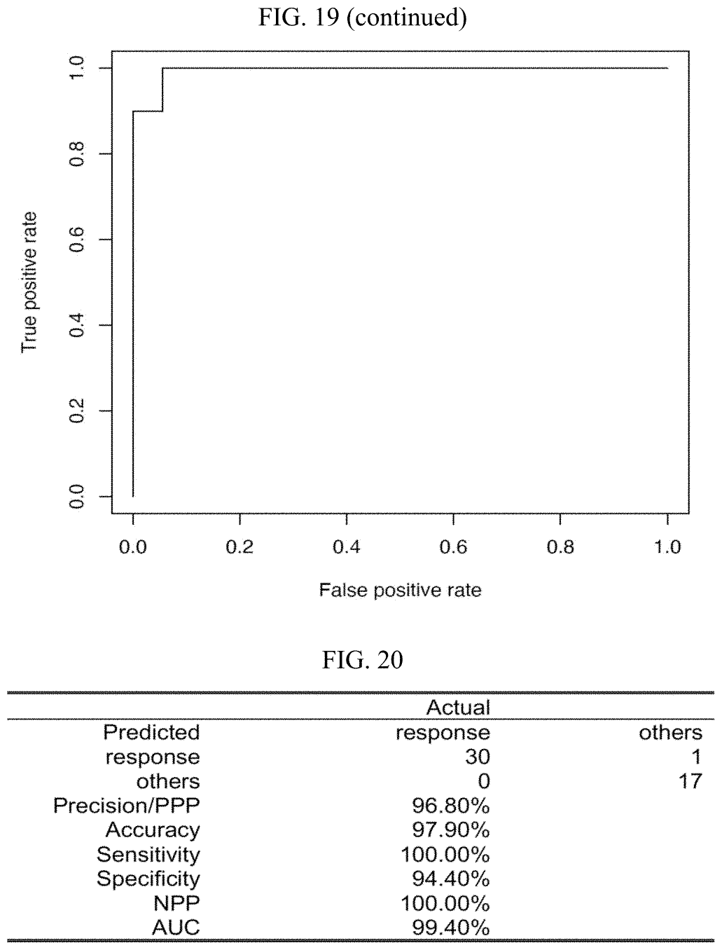

In regards to the Naproxen treatment arm, there were 30 responders and 18 others (adverse responder, unknown) when measuring change in MMSE score from baseline to 12 months. When the 20-protein predictive biomarker was applied from baseline samples, AUC reached 95% with a sensitivity of 1.00 and specificity of 0.78 (with an optimal cutoff-score of 0.69) (See FIG. 17 and FIG. 18). When utilizing proteomics at 12 months, the same 20-protein predictive biomarker algorithm reached an AUC of 99% with a sensitivity of 1.00 and specificity of 0.94 (with an optimal cut-off score of 0.254) (See FIG. 19 and FIG. 20).

### Selected Discussion

This data demonstrates, for the first time, that an endophenotype approach (i.e., the proinflammatory endophenotype) can predict treatment response in a prevention trial. That is, the proinflammatory endophenotype can be used to identify those cognitively normal older adults who a doctor can prevent dementia in over the next 5-years by prescribing an anti-inflammatory intervention.

### Example 7: A Precision Medicine Approach to Treating Alzheimer's Disease Using Rosiglitazone Therapy: A Biomarker Analysis of the REFLECT Trials

Alzheimer's disease (AD) is the most common neurodegenerative dementia. More than 5.7 million Americans suffer from this devastating disease. Every 65 seconds, an American develops AD, which is the 5th leading cause of death for those over the age of 65. AD has an annual healthcare cost similar to that of cardiovascular disease (CVD) and more than that of cancer. While death rates due to CVD and cancer have declined in recent decades, death rates due to AD have steadily increased, likely due to ineffective therapies. It was hypothesized that AD is a heterogeneous condition and, therefore, a paradigm shift is required to identify specific subpopulations for targeted precision medicine interventions. In fact, the complexity of AD may be the very key to addressing this devastating disease.

While much of today's pharmacotherapy is “trial and error,” precision medicine is a biomarker-guided medicine that is designed to improve early and accurate diagnostics and therapeutics. The FDA defines precision medicine (also known as “personalized medicine”) as “an innovative approach to tailoring disease prevention and treatment to take into account differences in people's genes, environments, and lifestyles. The goal of precision medicine is to target the right treatment to the right patients at the right time.” In fact, biomarker guided therapies in oncology have resulted in drastically improved patient outcomes. A precision medicine approach has been proposed for numerous diseases, including neurological diseases such as multiple sclerosis and AD. Despite the proposed use of precision medicine for AD, few studies to date have provided direct empirical support. By leveraging previously conducted clinical trial biorepositories, it is possible to provide proof-of-concept data for the precision medicine approach in AD.

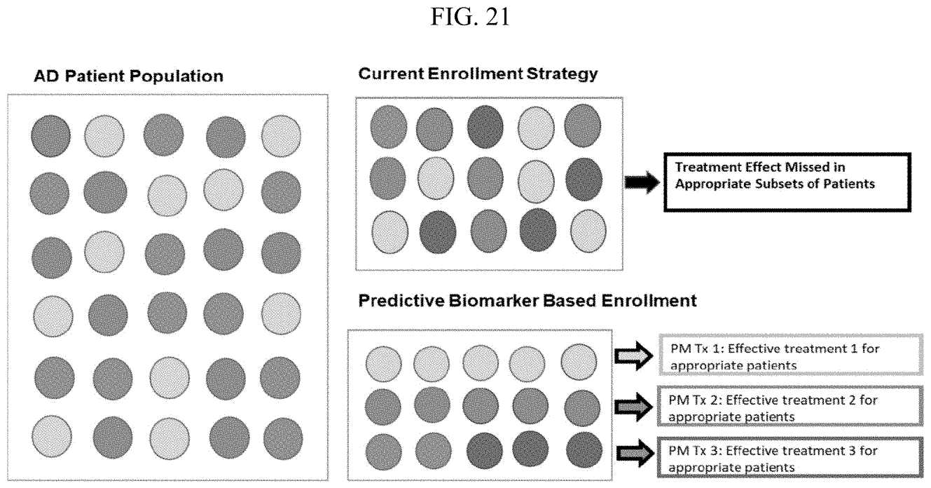

The FDA defines a “Predictive Biomarker” as “a biomarker used to identify individuals who are more likely than similar patients without the biomarker to experience a favorable or unfavorable effect from a specific intervention or exposure.” It is believed that the failure of clinical trials targeting AD is due to the fact that “most medical treatments are designed for the ‘average patient; as a one-size-fits-all approach.” This approach does not consider the substantial biological heterogeneity among patients. As seen in FIG. 21, it was hypothesized that there are multiple subgroups of patients within the larger AD patient population. Therefore, if “Treatment A” was appropriate and effective in only 20% of the population (or 1 subgroup), the clinical trial was doomed to fail as 80% of the patients selected were inappropriate. However, targeting that specific 20% of patients based on his/her biological dysfunction driving his/her dementia, optimal treatment outcomes can be seen that may have been impossible to find due to the trial design itself. Predictive biomarkers can be used to only enroll the specific group of patients most likely to benefit from the trial-specific intervention.

Peroxisome proliferator-activated receptor gamma (PPAR-γ) agonists are widely used for treatment of diabetes. PPAR-γ agonists such as rosiglitazone modulate many cellular processes, including several associated with AD through its reduction of tau and amyloid pathology and inhibition of inflammation (Govindarajulu, M. et al., “Signaling Mechanisms of Selective PPAR γ Modulators in Alzheimer's Disease,” PPAR Res., 2018, 2010675; Khan, M. A. et al., “Current Progress on Peroxisome Proliferator-activated Receptor Gamma Agonist as an Emerging Therapeutic Approach for the Treatment of Alzheimer's Disease: An Update,” Curr. Neuropharmacol., 2019, 17:232-246; Lin, C. H. et al., “Rosiglitazone rescues human neural stem cells from amyloid-beta induced ER stress via PPARγ dependent signaling,” Exp. Cell Res., 2018, 370:312-321). Rosiglitazone was examined in multiple trials (Phase 2 and Phase 3) as a potential treatment for mild-to-moderate AD in the REFLECT trials but these clinical trials did not meet clinical endpoints. However, it was hypothesized that the one-size-fits-all approach to the clinical trial design masked the therapeutic benefit experienced by a subset of patients. Methods were tested to create a predictive biomarker that identifies those specific AD patients that benefited from rosiglitazone therapy in the REFLECT trials.

### Methods

Participants and Methods for REFLECT Trials (Gold, M. et al., “Rosiglitazone monotherapy in mild-to-moderate alzheimer's disease: Results from a randomized, double-blind, placebo-controlled phase III study,” Dement. Geriatr. Cogn. Disord., 2010, 30:131-146; Harrington, C. et al., “Rosiglitazone Does Not Improve Cognition or Global Function when Used as Adjunctive Therapy to AChE Inhibitors in Mild-to-Moderate Alzheimer's Disease: Two Phase 3 Studies,” Curr. Alzheimer Res., 2011, 8:592-606)

The current study included samples and data from multiple trials of rosiglitazone therapy in AD including a Phase 2b (NCT00334568) study of 2 mg, 4 mg, and 8 mg. Three REFLECT trials included multiple studies of 2 mg or 8 mg rosiglitazone XR as a potential therapy for mild-to-moderate AD. REFLECT-1 (AVA105640; NCT00428090) was a 24-week, double-blind, double-dummy, randomized, parallel-group Phase III study. REFLECT-5 (AVA102677; NCT00550420) open-label extension of REFLECT-1. REFLECT-2 (Study AVA10267, NCT00348309) and REFLECT-3 (study AVA102670; NCT00348140), was a 52-week, randomized, double-blind, placebo-controlled, parallel-group study of rosiglitazone XR as an adjunctive therapy to ongoing acetylcholinesterase inhibitor (AChEI) treatment for 48 weeks. Participants who completed either study could then enroll into the open-label extension REFLECT-4 study for longer-term treatment. The sample size randomized per trial were as follows: REFLECT-1 n=581, REFLECT-2 n=1,496, and REFLECT-3 n=1,485. The samples and data from these trials were provided to the ADCS for academic research use and utilized for the purposes of this study. All clinical trials were conducted under IRB approved protocols and all patients or informants provided written informed consent. Due to funding limitations, only subsets of samples were assayed from each of the trials.

### Participant Screening Criteria for the REFLECT Trials

Inclusion criteria: Age 50-90 with a diagnosis of mild-to-moderate AD according to NINDS-ADRDA criteria, Mini Mental Status Examination (MMSE) score between 10-26 at screening and at least 6-months of ongoing donepezil or other approved AChEI therapy with stable dosing for at least 2 months prior to enrollment.

Exclusion criteria: Vascular dementia diagnosis; history or evidence of another cause of dementia; history of seizures; history of congestive heart failure; significant psychiatric illness that in the opinion of the investigator would interfere with the study; participants with controlled behavioral symptoms on stable doses of atypical antipsychotics, SSRIs, or anxiolytics were allowed; participants with untreated active major depressive disorder were excluded; type 1 and type 2 diabetes treated with insulin/PPAR-γ agonists/insulin secretagogues and agents with incretin effects were excluded; subjects with type 2 diabetes controlled by diet or exercise or metformin were allowed to enter the study if HbA1c <8.5% at screening.

### Proteomic Assays

All blood biomarker assays were conducted in the Institute for Translational Research (ITR) Biomarker Core.

Sample Preparation. Preparation of samples for proteomic assay was conducted using the Hamilton Robotics StarPlus system, which facilitates substantially improved quality of assays, increased QA/QC monitoring, as well as increased proteomic capacity in the laboratory. Any re-aliquoting was conducted via the Hamilton easyBlood robotic system.

Sample Assay. Plasma samples were assayed via multi-plex biomarker assay platform using electrochemiluminescence (ECL). All plasma samples were assayed for targeted markers of our proinflammatory endophenotype and metabolic endophenotype: c-reactive protein (CRP), interleukin (IL)-6, IL-10, tumor necrosis factor alpha (TNF-α), fatty acid binding protein (FABP)-3, and pancreatic polypeptide (PPY). Additional markers were assayed as part of the ITR Biomarker Core standard panel. The ITR laboratory has assayed over n>20,000 samples on these markers using this system. Inter- and intra-assay variability has been excellent. Average CVs (>3000 samples) for these assays are all <10% with the majority being <=5%.

### Statistical Analyses

The predictive biomarker profile was generated using support vector machine (SVM) analyses. SVM is based on the concept of decision planes that define decision boundaries and serves primarily as a classifier method by performing classification tasks through constructing hyperplanes in a multidimensional space that can separate cases of different class labels. SVM has the capacity to simultaneously take into account a large volume of data in order to generate an overall profile (e.g., over and under-expression of select proteins) that most accurately classifies multiple outcomes rather than only binary outcomes. As with all learning machine methods, a primary concern is for overfitting the data. In order to avoid this problem the following measures were taken: (1) restriction of the number of proteins included in the predictive model to a total of six pre-specified inflammatory and metabolic markers; (2) building of the predictive biomarker was based on responders versus non-responders (i.e. only 2 groups). Treatment responder was defined as an MMSE score that was stable or improved over trial duration whereas non-responder was defined as any decline in MMSE scores over the clinical trial duration. The goal of responder was to identify those who experience clinically meaningful outcomes rather than slowed decline. The purpose of this approach was to have a predictive biomarker that could selectively identify only those most likely to respond while all others would be ruled out; (3) an internal five-fold cross-validation was conducted within the sample with the SVM analyses. The SVM analyses were conducted with the e1071 package (v1.6-8) in R (v3.4.2). In order to build an SVM model to predict treatment response, the radial basis function kernel was used together with five-fold cross-validation, cost=100 and gamma=0.001. The original data was randomly partitioned into 5 equal sized subsamples. A single subsample was retained as a testing set while the remaining 4 subsamples were used as training sets. For each model, the cross-validation was run randomly five times.

Additionally, to avoid influence of outliers, all outliers beyond the fifth quintile were set at the fifth quintile. Finally, due to instability of assays at extremely low levels, any assay values below the standard curve were set at the least detectable limit for the assay. These approaches restricted any influence of outliers in any direction. SVM does not assume normality and, therefore, raw data were utilized. The analyses were restricted to rosiglitazone arms across trials as the goal was specifically to identify a predictive biomarker of treatment response. The SVM models were first generated by trial x arm and then by dosage combined across trials, where possible.

### Results

A total of 534 samples were assayed as part of this proof-of-concept study. Table 7 provide the descriptive statistics of the study population by clinical trial. First, the predictive biomarker was examined using pre-specified inflammatory and metabolic markers, which included IL-6, IL-10, CRP, TNFα, FABP-3, and PPY. These markers were used to predict treatment response versus non-response (based on change in MMSE score) within each clinical trial.

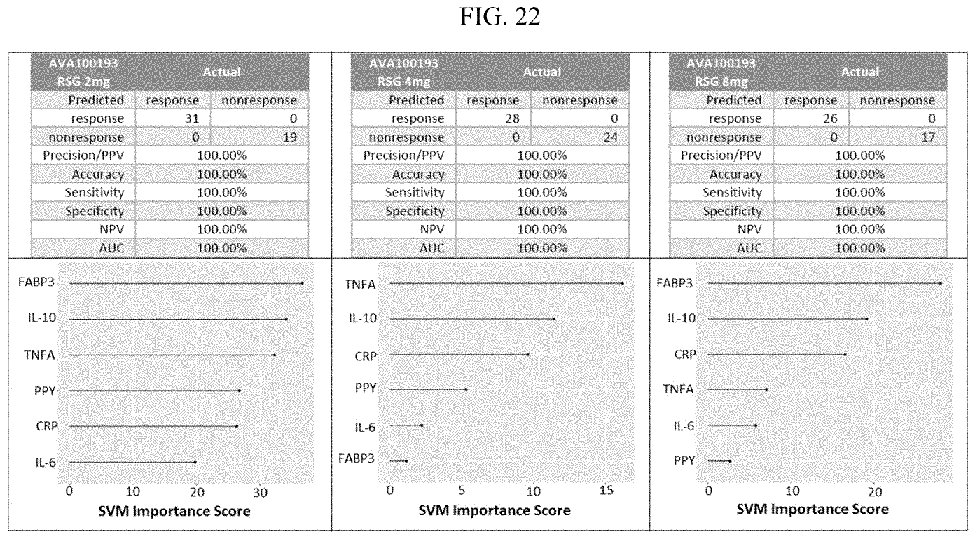

In the Phase 2 trial (AVA100193), there was a total of 31 responders and 19 non-responders in the 2 mg arm, 28 responders and 24 non-responders in the 4 mg arm and 26 responders and 17 non-responders in the 8 mg arm. Using the 6-protein algorithm, 100% of patients were correctly classified across study arms (FIG. 22).

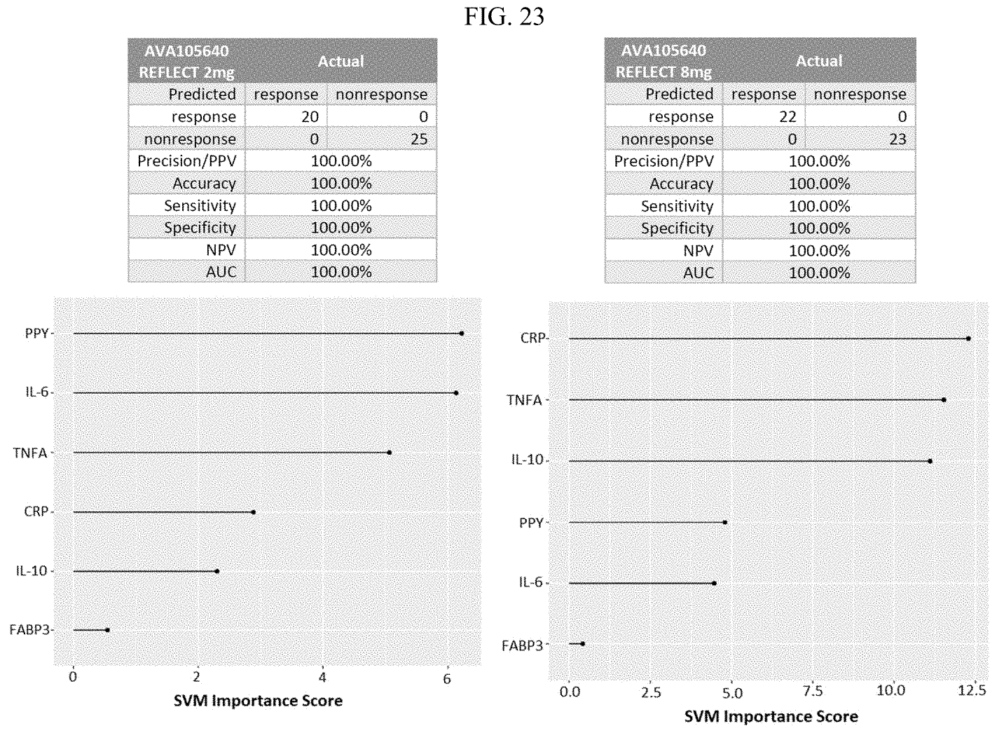

In the Phase 3 trial (AVA105640), there was 20 responders and 25 non-responders in the 2 mg XR group and 22 responders and 23 non-responders in the 8 mg XR group. Using the 6-protein predictive biomarker algorithm, 100% of the patients were correctly classified as responder or non-responder (FIG. 23).

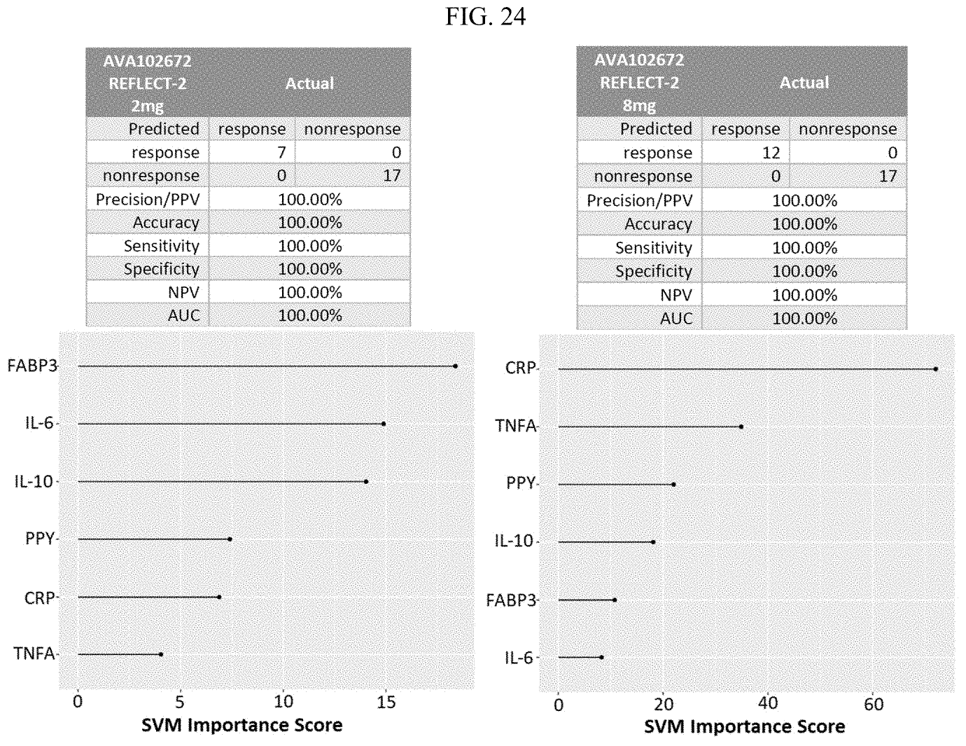

In the Phase 3 trial (AV102672), there was 7 responders and 17 non-responders in the 2 mg XR arm and 12 responders and 17 non-responders in the 8 mg XR arm. The 6-protein predictive biomarker algorithm was 100% accurate in identifying responders versus non-responders (FIG. 24).

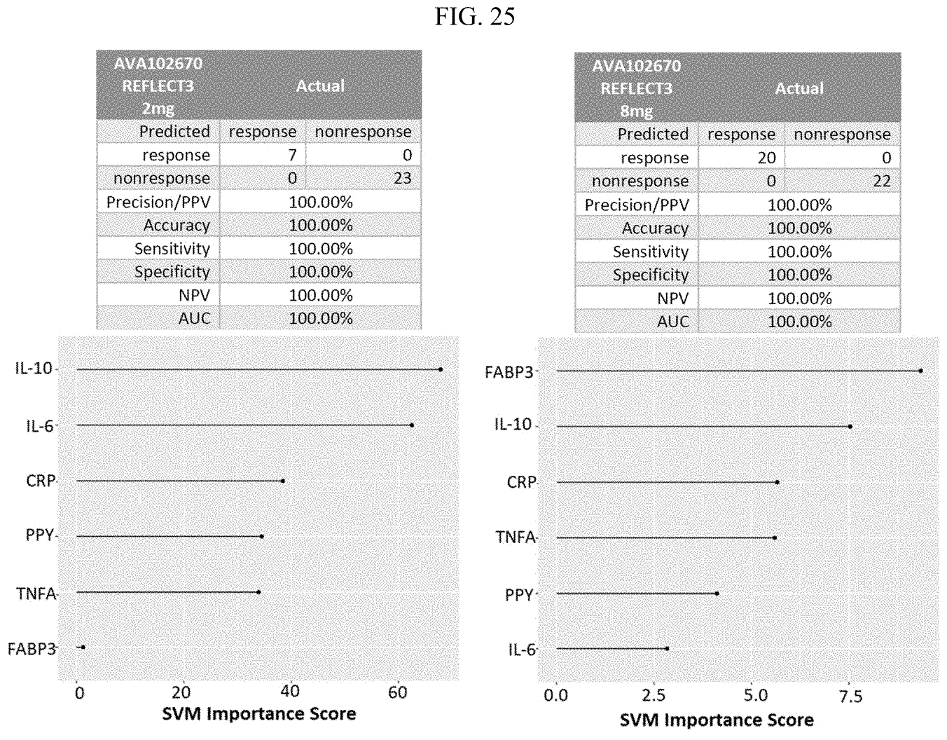

In the Phase 3 trial (AVA102670), there were 7 responders and 23 non-responders in the 2 mg XR arm and 20 responders and 22 non-responders in the 8 mg XR arm. The 6-protein predictive biomarker algorithm was 100% accurate in identifying responder versus non-responder (FIG. 25).

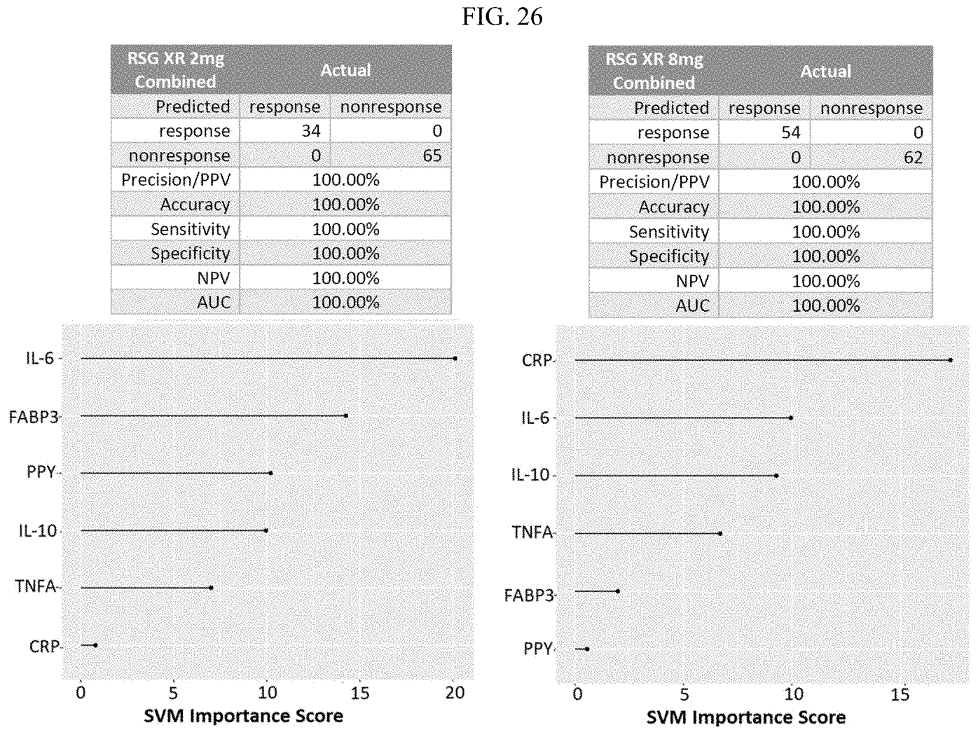

Next, data was combined across the 2 mg XR and 8 mg XR arms across trials. There were 34 responders and 65 non-responders in the 2 mg XR arm and 54 responders and 62 non-responders in the 8 mg XR arm. When the data was combined across these arms, the 6-protein predictive biomarker algorithm was again 100% accurate in identifying responders versus non-responders to rosiglitazone (FIG. 26).

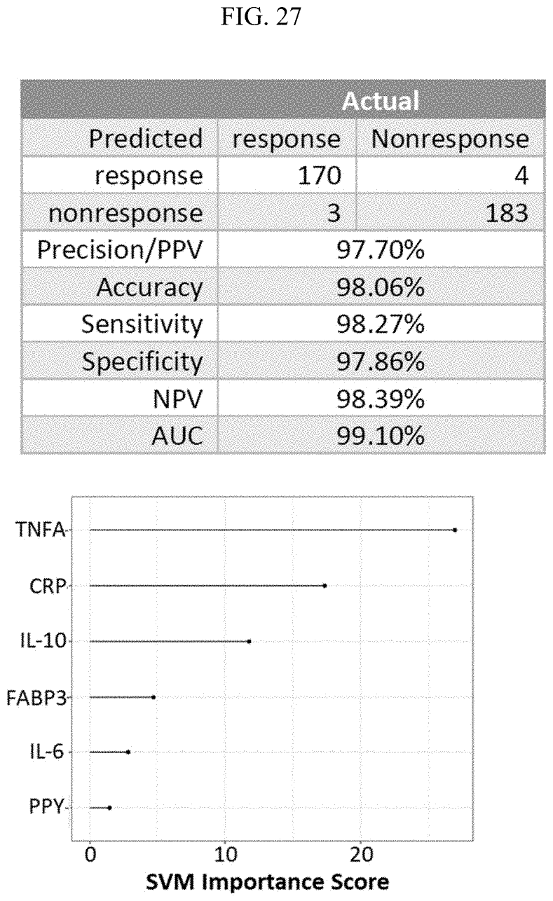

Finally, data was combined across all rosiglitazone therapy arms to determine if a global RSG-predictive biomarker could be generated. When combined across arms and trials, there were 173 responders and 187 non-responders. The 6-protein predictive biomarker was 98% accurate overall with 98% of treatment responders accurately classified (FIG. 27).

TABLE 7
All patients randomized and patients who are
responders and non-responders

| | Total | Responder | Non-responder |
|---|---|---|---|
| All patients | | | |
| No. randomized | 534 | 251 | 283 |
| Age percentiles | | | |
| 50 | 73 | 73 | 73 |
| 25, 75 | 67, 78 | 67.0, 78.5 | 67.5, 78.0 |
| 0, 100 | 50, 90 | 50, 90 | 50, 90 |
| Gender, % | | | |
| Female | 60.7 | 64.9 | 56.9 |
| Male | 39.3 | 35.1 | 43.1 |
| | Total | Responder | Non-responder |
| 2 mg RSG XR | | | |
| No. randomized | 99 | 34 | 65 |
| Age percentiles | | | |
| 50 | 73 | 74 | 73 |
| 25, 75 | 67.0, 78.5 | 67.0, 79.8 | 67, 78 |
| 0, 100 | 51, 89 | 55, 87 | 51, 89 |
| Gender, % | | | |
| Female | 53.5 | 67.6 | 46.2 |
| Male | 46.5 | 32.4 | 53.8 |
| | Total | Responder | Non-responder |
| 8 mg RSG XR | | | |
| No. randomized | 116 | 54 | 62 |
| Age percentiles | | | |
| 50 | 74 | 75 | 73 |
| 25, 75 | 67.8, 78.2 | 68.2, 79.0 | 65.2, 78.0 |
| 0, 100 | 50, 86 | 50, 86 | 51, 85 |
| Gender, % | | | |
| Female | 55.2 | 53.7 | 56.5 |
| Male | 44.8 | 46.3 | 43.5 |
| | Total | Responder | Non-responder |
| Donepezil (10 mg) | | | |
| No. randomized | 21 | 15 | 6 |
| Age percentiles | | | |
| 50 | 74 | 74 | 77.5 |
| 25, 75 | 70, 80 | 70.5, 79.0 | 67.2, 81.8 |
| 0, 100 | 59, 84 | 59, 84 | 62, 84 |
| Gender, % | | | |
| Female | 66.7 | 66.7 | 66.7 |
| Male | 33.3 | 33.3 | 33.3 |
| | Total | Responder | Non-responder |
| Placebo | | | |
| No. randomized | 153 | 63 | 90 |
| Age percentiles | | | |
| 50 | 74 | 73 | 75 |
| 25, 75 | 68, 79 | 67, 80 | 69.0, 78.8 |
| 0, 100 | 50, 90 | 52, 90 | 50, 90 |
| Gender, % | | | |
| Female | 69.9 | 74.6 | 66.7 |
| Male | 30.1 | 25.4 | 33.3 |
| | Total | Responder | Non-responder |
| RSG 2 mg | | | |
| No. randomized | 50 | 31 | 19 |
| Age percentiles | | | |
| 50 | 72 | 72 | 72 |
| 25, 75 | 67, 75 | 67.0, 75.5 | 68.5, 74.5 |
| 0, 100 | 50, 83 | 54, 83 | 50, 81 |
| Gender, % | | | |
| Female | 64 | 64.5 | 63.2 |
| Male | 36 | 35.5 | 36.8 |
| | Total | Responder | Non-responder |
| RSG 4 mg | | | |
| No. randomized | 52 | 28 | 24 |
| Age percentiles | | | |
| 50 | 72.5 | 70 | 73.5 |
| 25, 75 | 59.8, 76.2 | 58.8, 75.2 | 62.2, 77.5 |
| 0, 100 | 52, 83 | 52, 82 | 53, 83 |
| Gender, % | | | |
| Female | 51.9 | 57.1 | 45.8 |
| Male | 48.1 | 42.9 | 54.2 |
| | Total | Responder | Non-responder |
| RSG 8 mg | | | |
| No. randomized | 43 | 26 | 17 |
| Age percentiles | | | |
| 50 | 72 | 72 | 71 |
| 25, 75 | 66.0, 75.5 | 65.5, 75.0 | 68, 77 |
| 0, 100 | 53, 84 | 54, 83 | 53, 84 |
| Gender, % | | | |
| Female | 62.8 | 69.2 | 52.9 |
| Male | 37.2 | 30.8 | 47.1 |

### Selected Discussion

The current data suggests that a 6-protein algorithm consisting of markers covering inflammatory and metabolic pathways can be utilized to generate a predictive biomarker that can be used to identify those AD patients most likely to benefit from 2 mg, 4 mg, and 8 mg rosiglitazone therapy. Therefore, the current findings offer additional proof-of-concept support for a precision medicine approach to targeted treatment among specific subsets of patients suffering from AD. Two subgroups of patients have been demonstrated that could be screened from the population of AD patients and enrolled into targeted therapeutic trials for optimal benefit from NSAID or rosiglitazone therapies.

A targeted panel of markers can be utilized to generate a predictive biomarker for the identification of specific subsets of AD patients who would most likely benefit from specified therapies. Individuals who met enrollment criteria with a diagnosis of probable AD in this trial were randomized to rofecoxib (25 mg once daily), naproxen (220 mg twice-daily) or placebo. In this study, the inflammatory-specific predictive biomarker was 97% accurate in identifying treatment response to naproxen and 98% accurate in identifying treatment response to rofecoxib.

In the present study, a specific a priori defined set of metabolic and inflammatory markers were examined. Rosiglitazone has well-documented anti-inflammatory and neuroprotective qualities. Therefore, it was hypothesized that combining both metabolic and inflammatory markers into the predictive biomarker would yield optimal success. Additional markers were assayed for further refinement; however, such markers did not need to be considered given the overall accuracy of the profile. Combined, this data indicates that interventions targeting the metabolic pathway (such as rosiglitazone) may need to be ethnically-tailored as vascular factors were more predictive of N among non-Hispanic whites.

There are multiple substantial benefits of the precision medicine approach for enrolling patients using predictive biomarkers into novel trials as outlined here. First, by enrolling only those patients most likely to benefit the effect size of the trial increases and, therefore, the sample size decreases substantially. Second, by screening for multiple subgroups in the AD patient population, multiple trials can be enrolled simultaneously (FIG. 21) thereby reducing cost and patient burden. Finally, the predictive biomarker approach increases likelihood of success of trials and, therefore, can expedite novel therapeutic interventions to market thereby providing patients novel treatments sooner and companies extended patent life. A non-limiting goal with this work is as follows: (1) provide rapid, scalable tests that can be deployed to primary care doctors in order to arm them with knowledge that could foster treatment opportunities using readily available drugs, and (2) conduct these analyses in collaboration with novel compounds beginning early (pre-clinical) in order to generate drug-specific companion diagnostics that could be used to streamline clinical trials, reduce trial costs, and increase trial effect sizes. Therefore, it is the precision medicine model needs to be further investigated both using existing biorepository samples as well as in new prospective clinical trials.

### ENUMERATED EMBODIMENTS

The following exemplary embodiments are provided, the numbering of which is not to be construed as designating levels of importance:

Embodiment 1 provides a method for selecting a therapy for improved cognition or to prevent cognitive decline or dysfunction comprising:

obtaining a sample from a subject;

screening the sample to measure one or more biomarkers or scores for a proinflammatory, a metabolic, and/or a neurotrophic endophenotype profile;

identifying the proinflammatory, metabolic, and/or neurotrophic endophenotype profile based on the biomarkers and level of expression of the biomarkers identified that are associated with cognitive decline or dysfunction when compared to the level of expression of the biomarkers within a sample of patients suffering from cognitive loss; and selecting a course of treatment for the subject based on whether the subject is selected as having the proinflammatory, metabolic, and/or neurotrophic endophenotype profile that is associated with cognitive decline or dysfunction.

Embodiment 2 provides the method of Embodiment 1, wherein the endophenotype profile is generated using learning machines (random forest, support vector machines), clustering algorithms (factor analysis, principal component analysis), summation of values, or other methods to generate an endophenotype profile score across multiple measures.

Embodiment 3 provides the method of Embodiment 1, wherein the sample is a blood or plasma sample, and wherein at least one of the biomarker measurements is obtained by a method selected from an immunoassay, an enzymatic activity assay, fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling.

Embodiment 4 provides the method of Embodiment 1, further comprising the step of generating an endophenotype profile dataset that comprises the level of the one or more biomarkers prior to the step of comparing the level of the one or more biomarkers within a sample of patients suffering from cognitive loss.

Embodiment 5 provides the method of Embodiment 1, wherein the cognitive decline or dysfunction is a disease or condition selected from mild cognitive impairment, Alzheimer's Disease, Parkinson's Disease, Down's syndrome, Frontotemporal dementia, Dementia with Lewy Bodies, Multiple sclerosis, traumatic brain injury, depression, schizophrenia, bipolar disease (and other mental illness), diabetes, hypertension, stroke, heart attack, dyslipidemia, other cognitive conditions/diseases, or aging.

Embodiment 6 provides the method of Embodiment 1, further comprising the steps of obtaining one or more additional blood samples from the patient after a pre-determined amount of time and comparing the levels of the biomarkers from the one or more additional samples to determine progression of cognitive loss.

Embodiment 7 provides the method of Embodiment 1, further comprising the steps of treating the patient for a pre-determined period of time, obtaining one or more additional blood samples from the patient after the pre-determined amount of time and comparing the levels of the biomarkers from the one or more additional samples to determine progression of cognitive loss or effectiveness of the therapy.

Embodiment 8 provides the method of Embodiment 1, further comprising the steps of selecting one or more anti-inflammatory therapies for improved cognition or to reduce cognitive loss based on a proinflammatory endophenotype comprising:

generating a high and a low proinflammatory endophenotype by determining the level of expression of two or more biomarkers in the sample selected from interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (FVII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, or α-synuclein; and more particularly 3, 4, 5, 6, 7, 8, 9, 10, 11, 12, 13, 14, 15, 16, 17, 18, 19, 20, 21, 22, 23, 24, or 25 biomarkers selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, Aβ, tau, or α-synuclein; and

determining a high or a low proinflammatory endophenotype profile by determining the level of expression of the two or more biomarkers of a high or a low proinflammatory endophenotype; and

selecting a therapy that is indicated for the proinflammatory endophenotype profile or not selecting a therapy that is contraindicated for the proinflammatory endophenotype profile.

Embodiment 9 provides the method of Embodiment 1, further comprising the steps of selecting one or more anti-diabetic therapies for improved cognition or to reduce cognitive loss based on a metabolic endophenotype comprising:

generating a high and a low metabolic endophenotype by determining the level of expression of two or more biomarkers in the sample selected from alpha-2-macroglobulin (A2M), fatty acid binding protein (FABP), pancreatic polypeptide (PPP), glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, cholesterol, body mass index (BMI), or waist circumference; and more particularly 3, 4, 5, 6, 7, 8, 9, 10, 11, 12, 13, 14, 15, 16, 17, 18 or 19 biomarkers selected from alpha-2-macroglobulin (A2M), fatty acid binding protein (FABP), pancreatic polypeptide (PPP), glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, cholesterol, body mass index (BMI), or waist circumference;

dividing the level of expression of the two or more markers as being either high metabolic endophenotype or low metabolic endophenotype; and

selecting a course of treatment for the subject based on whether the subject is selected as being high metabolic endophenotype or low metabolic endophenotype, wherein a high metabolic endophenotype subject benefits from a treatment with an anti-diabetic drug.

Embodiment 10 provides the method of Embodiment 1, further comprising the steps of selecting a therapy for improved cognition, or prevention of cognitive dysfunction/loss using one or more anti-diabetic therapies for subjects of Mexican-American ethnogenicity comprising:

obtaining a sample from a Mexican-American subject;

generating a high and a low metabolic endophenotype by determining the level of expression of two or more markers selected from fatty acid binding protein (FABP), CD40, glucagon like protein-1 (GLP-1), IgM, beta-2 microglobulin, IGF-binding protein 2, IL-8, peptide YY, macrophage derived chemokine (MDC), macrophage inflammatory protein-1 (MIP-1 alpha), pancreatic polypeptide, glycated hemoglobin A1c (HbA1c), glucose, triglycerides, high density lipoprotein (HDL), low density lipoprotein (LDL and vLDLs), diacylglycerol acyl-transferase 1 (DGAT1), peroxisome proliferator-activated receptor (PPAR)-γ, PPARα, cholesterol, body mass index (BMI), or waist circumference;

dividing the level of expression of the two or more markers as being either high metabolic endophenotype or low metabolic endophenotype; and

selecting a course of treatment for the subject based on whether the subject is selected as being high metabolic endophenotype or low metabolic endophenotype, wherein a high metabolic endophenotype subject benefits from a treatment with an anti-diabetic drug.

Embodiment 11 provides the method of Embodiment 1, further comprising the steps of selecting one or more therapies for improved cognition or prevention of cognitive loss based on the neurotrophic endophenotype comprising:

obtaining a sample from a subject;

generating a high and a low neurotrophic endophenotype by determining the level of expression of 2, 3, 4, 5, 6, or 7 biomarkers selected from brain derived neurotrophic factor (BDNF), nerve growth factor (NGF), tenascin 3 (TN-3), ciliary neurotrophic factor (CNTF), glial cell derived neurotrophic factor (GDNF), leukemia inhibitory factor (LIF), and neuregulin-1 (GGF), wherein the high and low neurotrophic groupings by determining the level of expression of the 2, 3, 4, 5, 6, or 7 biomarkers;

dividing the level of expression of the two or more biomarkers as being either high neurotrophic endophenotype or low neurotrophic endophenotype; and

selecting a course of treatment for the subject based on whether the subject is selected as being high neurotrophic endophenotype or low neurotrophic endophenotype.

Embodiment 12 provides the method of Embodiment 6, wherein the high proinflammatory endophenotype may be treated with one of more of the following therapeutic agents: NSAIDs, non-selective NSAIDs, selective NSAIDs, steroids, glucocorticoids, Immune Selective Anti-Inflammatory Derivatives (ImSAIDs), anti-TNF medications, anti-IL5 drugs or CRP-lowering agents.

Embodiment 13 provides the method of Embodiment 6, wherein one or more of the following therapeutic agents: NSAIDs, non-selective NSAIDs, selective NSAIDs, steroids, glucocorticoids, Immune Selective Anti-Inflammatory Derivatives (ImSAIDs), anti-TNF medications, anti-IL5 drugs or CRP-lowering agents are contraindicated if the subject does not have a high proinflammatory endophenotype.

Embodiment 14 provides the method of Embodiment 1, wherein the endophenotype profile is identified as a metabolic endophenotypes and wherein if the subject is scored in the high metabolic endophenotype profile an anti-diabetic treatment is indicated, and if the subject is scored in a low metabolic dysfunction group then an anti-diabetic treatment is contraindicated.

Embodiment 15 provides the method of Embodiment 14, wherein the high metabolic endophenotype profile is treated with one of more of the following therapeutic agents: with anti-diabetic, insulin, GLP-1 medications, amylin-related medications, or oral hypoglycemic.

Embodiment 16 provides the method of Embodiment 1, wherein the endophenotype profile is identified as a neurotrophic endophenotype profile that is treated with one of more of the following therapeutic agents: neurotrophic factor agonist, exercise therapy, BDNF and BDNF agonists, selective serotonin reuptake inhibitors, selective serotonin 2C (5-HT2C) antagonists, serotonin-norepinephrine reuptake inhibitors, tricyclic, combined exercise and medications, GDNF and GDNF agonists.

Embodiment 17 provides the method of Embodiment 1, wherein the step of identifying the endophenotype profile further comprises:

determining the tertile of the level of expression of two or more biomarkers; and

depending on the level of expression dividing the level of expression of the two or more biomarkers or scores as being either high or low proinflammatory, metabolic, and/or neurotrophic endophenotype profile;

selecting a course of treatment for the subject based on whether the subject is selected as being high or low proinflammatory, metabolic, and/or neurotrophic endophenotype profile,

wherein the tertile is determined by: scoring the tertile scores for the two or more biomarkers to generate a score; and

assigning a lower score to the low end of the proinflammatory, metabolic, and/or neurotrophic endophenotype profile, or

assigning a higher score to the high end of the proinflammatory, metabolic, and/or neurotrophic endophenotype profile, with all other scores falling in a middle score.

Embodiment 18 provides a method of determining the effectiveness of a candidate drug for treating or preventing cognitive loss, the method comprising:

(a) measuring two or more biomarkers for a proinflammatory, a metabolic, and/or a neurotrophic endophenotype profile;

(b) administering the candidate drug to a first subset of the patients, and a placebo to a second subset of the patients;

(c) generating a proinflammatory, a metabolic, and/or a neurotrophic endophenotype profile dataset using one or a combination of the one or more biomarkers for the first and second subset of patients;

(d) determining the tertile of the level of expression of the one or more biomarkers; and depending on the level of expression dividing the level of expression of the one or more biomarkers as being either high or low proinflammatory, metabolic, and/or neurotrophic endophenotype profile;

(e) determining if a baseline high or low proinflammatory, metabolic, and/or neurotrophic endophenotype profile group predicted treatment response such that the high proinflammatory, metabolic, and/or neurotrophic, endophenotype profile group responded differentially than the low proinflammatory, metabolic, and/or neurotrophic endophenotype profile group;

(f) repeating step (a) after the administration of the candidate drug or the placebo;

(g) determining if the candidate drug modifies the proinflammatory, metabolic, and/or neurotrophic endophenotype profile over the course of the trial; and

(h) determining if change in the proinflammatory, metabolic, and/or neurotrophic endophenotype profile over the course of the trial predicted a positive response, a negative response, or a no treatment response, and if a statistically significant treatment response for cognitive loss, cognitive improvement or stability of cognitive functioning with the candidate drug is obtained, wherein a change in the proinflammatory, metabolic, and/or neurotrophic, profile is indicative of the candidate drug having effectiveness.

Embodiment 19 provides a method for selecting a therapy for improved cognition and/or prevention of cognitive dysfunction/loss using one or more endophenotypes comprising:

obtaining a sample from a subject;

measuring biomarkers or scores that differentiate between a proinflammatory, a metabolic, and a neurotrophic endophenotype to identify the cognitive dysfunction/loss using one or more endophenotypes; and

selecting a course of treatment for the subject based on whether the subject is scored as having a high or a low endophenotype for one or more of the proinflammatory, a metabolic, and/or a neurotrophic endophenotypes.

Embodiment 20 provides an apparatus for selecting a therapy for improved cognition as well as prevention of cognitive dysfunction/loss using one or more endophenotypes comprising:

a biomarker array that detects biomarkers from a sample for two or more endophenotypes selected from a proinflammatory, a metabolic, and/or a neurotrophic endophenotype profile;

a processor that obtains a biomarker expression output from the biomarker array, wherein an endophenotype profile is generated using learning machines (random forest, support vector machines), clustering algorithms (factor analysis, principal component analysis), summation of values, or other methods to generate an endophenotype score across multiple measures; and

an output that indicates a course of treatment for the subject based on whether the subject is scored as having a high or a low endophenotype for at least one of the proinflammatory, metabolic, or neurotrophic endophenotypes.

Embodiment 21 provides a method for selecting patients to determine the effectiveness of a candidate drug comprising:

generating a prediction model dataset by:
pre-selecting a level of treatment response selected from positive, negative and no response for a patient dataset within an endophenotype profile, wherein the endophenotypes are selected from at least one of a proinflammatory, a metabolic, or a neurotrophic endophenotype profile;
obtaining the patient dataset based on the endophenotype profile selected; and separating the patient dataset into a responder patient dataset, non-responder patient dataset and adverse responder patient dataset;

applying the prediction model blindly to a second clinical trial dataset to predict outcomes; and

determining the efficacy of the prediction model in predicting treatment responders, non-responders and adverse responders in a third trial, wherein the efficacy for the third trial is increased by only evaluating a patient outcome from the responder patient dataset.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Gottesman, I I, Shields J. Genetic theorizing and schizophrenia. British Journal of Psychiatry. 1973; 122(566):15-30.
2. Gottesman, I I, Gould T D. The endophenotype concept in psychiatry: etymology and strategic intentions. American Journal of Psychiatry. 2003; 160(4):636-645.
3. O'Bryant S E, Waring S C, Hobson V, et al. Decreased C-reactive protein levels in alzheimer disease. Journal of Geriatric Psychiatry and Neurology. 2010; 23(1):49-53.
4. O'Bryant S E, Xiao G, Barber R, et al. A serum protein-based algorithm for the detection of Alzheimer disease. Archives of Neurology. 2010; 67(9):1077-1081.
5. O'Bryant S E, Hobson V L, Hall J R, et al. Serum Brain-Derived Neurotrophic Factor Levels Are Specifically Associated with Memory Performance among Alzheimer's Disease Cases. Dementia and Geriatric Cognitive Disorders. 2010; 31(1):31-36.
6. Johnson L A, Hall J R, O'Bryant S E. A Depressive Endophenotype of Mild Cognitive Impairment and Alzheimer's Disease. PLoS ONE. 2013; 8(7):e68848.
7. O'Bryant S E, Xiao G, Edwards M, et al. Biomarkers of Alzheimer's disease among Mexican Americans. Journal of Alzheimer's Disease. 2013; 34(4):841-849.
8. Janocko N J, Brodersen K A, Soto-Ortolaza A I, et al. Neuropathologically defined subtypes of Alzheimer's disease differ significantly from neurofibrillary tangle-predominant dementia. Acta Neuropathologica. 2012:1-12.
9. Braskie M N, Ringman J M, Thompson P M. Neuroimaging measures as endophenotypes in Alzheimer's disease. International Journal of Alzheimer's Disease. 2011.
10. During E H O R, Elahi F M, Mosconi L, de Leon, M J. The concept of FDG-PET endophenotype in Alzheimer's disease. Neurol Sci. 2011; 32:559-569.
11. N E-T. Gene expression endophenotypes: a novel approach for gene discovery in Alzheimer's disease. Molecular Neurodegeneration. 2011; 3(31):1-14.
12. Cruchaga C K J, Nowotny P, Bales K, Pickering E H, Mayo K, Bertelsen S, Hinrichs A, the ADNI initiative, Fagan A M, Holtzman D M, Morris J C, and Goate A M. Cerebrospinal fluit APOE levels: an endophenotype for genetic studies for Alzheimer's disease. Human Molecular Genetics. 2012; 2012.
13. O'Bryant S E, Hobson V, Hall J R, et al. Brain-derived neurotrophic factor levels in alzheimer's disease. Journal of Alzheimer's Disease. 2009; 17(2):337-341.
14. O'Bryant S E, Hobson V L, Hall J R, et al. Serum brain-derived neurotrophic factor levels are specifically associated with memory performance among Alzheimer's disease cases. Dementia and Geriatric Cognitive Disorders. 2011; 31(1):31-36.
15. O'Bryant S E, Xiao G, Barber R, et al. A blood-based algorithm for the detection of Alzheimer's disease. Dementia and Geriatric Cognitive Disorders. 2011; 32(1):55-62.
16. O'Bryant S E, Xiao G, Barber R, et al. A Blood-Based Screening Tool for Alzheimer's Disease That Spans Serum and Plasma: Findings from TARC and ADNI. PLoS ONE. 2011; 6(12):e28092.

What is claimed is:

1. A method of treating a subject to improve cognition or to prevent cognitive decline or dysfunction in the subject, the method comprising:
obtaining a blood, plasma, or serum sample from the subject;
measuring in the sample the expression level of one or more proinflammatory biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY);
determining that the subject has a proinflammatory endophenotype profile based on the expression level of the proinflammatory biomarkers; and
administering an NSAID selected from naproxen and celecoxib to the subject.

2. The method of claim 1, wherein the subject is treated to improve cognition or to prevent cognitive decline or dysfunction related to memory loss, senility, dementia, Alzheimer's disease, or a combination thereof.

3. The method of claim 1, wherein the proinflammatory endophenotype profile is determined by comparing the expression level of one or more biomarkers in the blood, plasma, or serum sample obtained from the subject to the average expression level of the one or more corresponding biomarkers from a statistical sample representative of the subject.

4. The method of claim 3, further comprising one of steps (a)-(d)
(a) determining that the blood, plasma, or serum sample obtained from the subject has a statistically different expression level of each of IL-10, eotaxin 3, A2M, and SAA, and optionally Tenacin C when compared to a group of individuals in the statistical sample who do not have a family history of memory loss, senility, dementia, Alzheimer's disease, or a combination thereof; and administering celecoxib to the subject;
(b) determining that the blood, plasma, or serum sample obtained from the subject has a statistically similar expression level of each of IL-10, eotaxin 3, A2M, and SAA, and optionally Tenacin C when compared to a group of individuals in the statistical sample who have a family history of memory loss, senility, dementia, Alzheimer's disease, or a combination thereof; and administering celecoxib to the subject;
(c) determining that the blood, plasma, or serum sample obtained from the subject has a statistically different expression level of each of TPO, IL-5, IL-7, and I309, and optionally IL-6 when compared to a group of individuals in the statistical sample who do not have a family history of memory loss, senility, dementia, Alzheimer's disease, or a combination thereof; and administering naproxen to the subject; or
(d) determining that the blood, plasma, or serum sample obtained from the subject has a statistically similar expression level of each of TPO, IL-5, IL-7, and I309, and optionally IL-6 when compared to a group of individuals in the statistical sample who have a family history of memory loss, senility, dementia, Alzheimer's disease, or a combination thereof; and administering naproxen to the subject.

5. The method of claim 1, further comprising at least one of (a) or (b):
(a) obtaining a result of one or more neurocognitive evaluations from the subject before administration of the NSAID;
obtaining the result of the corresponding one or more neurocognitive evaluations from the subject after administration of the NSAID; and
comparing the results of the one or more neurocognitive evaluations before administration of the NSAID with those after administration of the NSAID; or
(b) obtaining a blood, plasma, or serum sample from the subject after administration of the NSAID;
measuring in the sample the expression level of the corresponding one or more proinflammatory biomarkers; and
comparing the expression level of the one or more biomarkers in the blood, plasma, or serum sample before administration of the NSAID with those after administration of the NSAID.

6. The method of claim 5, wherein the one or more neurocognitive evaluations are selected from the group consisting of a 4-point clock drawing test, a verbal fluency test, a trail making test, a list learning test, a mini-mental state exam, and combinations thereof.

7. The method of claim 1, wherein the step of administering an NSAID selected from naproxen and celecoxib to the subject comprises administering between about 175-225 mg of naproxen or celecoxib to the subject between once and eight times a day.

8. A method of determining a surrogate outcome of an NSAID clinical trial to improve cognition or to prevent cognitive decline or dysfunction, the method comprising:
(a) selecting a subject to participate in the clinical trial, wherein the subject is suspected of having age-related memory loss, senility, dementia, or Alzheimer's Disease, or having an elevated risk of age-related memory loss, senility, dementia, or Alzheimer's Disease;
(b) measuring in a blood, plasma, or serum sample obtained from the subject the expression level of one or more biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY);
(c) administering an NSAID selected from celecoxib and naproxen to a first subset of subjects and a placebo to a second subset of subjects;
(d) repeating step (b) after the administration of the NSAID or the placebo; and
(e) determining if a statistically significant change in the expression level of the one or more biomarkers is seen in the first subset of subjects when compared to any change occurring in the second subset of subjects, wherein a statistically significant change in the expression level is associated with a beneficial long term clinical outcome.

9. The method of claim 8, wherein when the NSAID administered in the clinical trial is celecoxib, the expression level of each of the biomarkers FABP, tenacin C, B2M, and TPO, and optionally IL-5 is measured in steps (b) and (d); and when the NSAID administered in the clinical trial is naproxen, the expression level of each of the biomarkers TPO, IL-6, CRP, and FABP, and optionally Factor VII is measured in steps (b) and (d).

10. A method of treating an Alzheimer's disease patient to improve cognition or to prevent cognitive decline or dysfunction in the patient, the method comprising:
obtaining a blood, plasma, or serum sample from the patient;
measuring in the sample the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol;
determining that the patient has both a proinflammatory endophenotype profile and a metabolic endophenotype profile based on the expression level of the one or more biomarkers; and
administering a PPAR-γ agonist to the patient.

11. The method of claim 10, wherein the patient is determined to have both a proinflammatory endophenotype profile and a metabolic endophenotype profile by comparing the expression level of the one or more biomarkers in the blood, plasma, or serum sample obtained from the patient to the average expression level of the one or more corresponding biomarkers from a statistical sample representative of the patient.

12. The method of claim 11,
wherein a statistically different expression level in the blood, plasma, or serum sample of one or more biomarkers selected from IL-6, IL-10, CRP, TNFα, FABP and PPY when compared to the expression level of the one or more corresponding biomarkers from individuals in the statistical sample who do not have Alzheimer's disease indicates that the patient has both a proinflammatory endophenotype profile and a metabolic endophenotype profile; or
wherein a statistically similar expression level in the blood, plasma, or serum sample of the one or more biomarkers selected from IL-6, IL-10, CRP, TNFα, FABP and PPY when compared to the expression level of the one or more corresponding biomarkers from individuals in the statistical sample who have been diagnosed with Alzheimer's disease indicates that the patient has both a proinflammatory endophenotype profile and a metabolic endophenotype profile.

13. The method of claim 11,
wherein a statistically different expression level in the blood, plasma, or serum sample of each of IL-6, IL-10, CRP, TNFα, FABP and PPY when compared to the expression level of each of the corresponding biomarkers from individuals in the statistical sample who do not have Alzheimer's disease indicates that the patient has both a proinflammatory endophenotype profile and a metabolic endophenotype profile; or
wherein a statistically similar expression level in the blood, plasma, or serum sample of each of IL-6, IL-10, CRP, TNFα, FABP and PPY when compared to the expression level of each of the corresponding biomarkers from individuals in the statistical sample who have been diagnosed with Alzheimer's disease indicates that the patient has both a proinflammatory endophenotype profile and a metabolic endophenotype profile.

14. The method of claim 10, wherein the PPAR-γ agonist is selected from rosiglitazone and rosiglitazone XR.

15. The method of claim 10, wherein the step of administering a PPAR-γ agonist to the patient comprises administering between about 2-10 mg of a PPAR-γ agonist is selected from rosiglitazone and rosiglitazone XR to the patient between once and eight times a day.

16. The method of claim 11, further comprising
measuring a body mass index (BMI) and/or waist circumference of the patient;
determining that the BMI and/or waist circumference of the patient is higher when compared to the average BMI and/or waist circumference from individuals in the statistical sample who do not have Alzheimer's disease, or determining that the BMI and/or waist circumference of the patient is higher when compared to the average BMI and/or waist circumference from individuals in the statistical sample who have been diagnosed with Alzheimer' s disease;
comparing the expression level of one or more biomarkers selected from the group consisting of IL-6, IL-10, CRP, TNFα, FABP, and PPY in the blood, plasma, or serum sample obtained from the patient to the average expression level of the one or more corresponding biomarkers from a statistical sample representative of the patient; and
determining that the patient has both a proinflammatory endophenotype profile and a metabolic endophenotype profile.

17. The method of claim 10, further comprising at least one of (a) or (b):
(a) obtaining a result of one or more neurocognitive evaluations from the patient before administration of a PPAR-γ agonist;
obtaining the result of the corresponding one or more neurocognitive evaluations from the patient after administration of the PPAR-γ agonist; and
comparing the results of the one or more neurocognitive evaluations before administration of the PPAR-γ agonist with those after administration of the PPAR-γ agonist;
(b) obtaining a blood, plasma, or serum sample from the patient after administration of the PPAR-γ agonist;
measuring in the sample the expression level of the corresponding one or more biomarkers; and
comparing the expression level of the one or more biomarkers in the blood, plasma, or serum sample before administration of the PPAR-γ agonist with those after administration of the PPAR-γ agonist.

18. The method of claim 17, wherein the one or more neurocognitive evaluations are selected from the group consisting of a 4-point clock drawing test, a verbal fluency test, a trail making test, a list learning test, a mini-mental state exam, and combinations thereof.

19. A method of determining a surrogate outcome of a PPAR-γ agonist clinical trial to improve cognition or to prevent cognitive decline or dysfunction, the method comprising:
(a) selecting a subject to participate in the clinical trial, wherein the subject is suspected of having age-related memory loss, senility, dementia, or Alzheimer's Disease, or having an elevated risk of age-related memory loss, senility, dementia, or Alzheimer's Disease;
(b) measuring, in a blood, plasma, or serum sample obtained from the subject, the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol;
(c) determining that the subject has both a proinflammatory endophenotype profile and a metabolic endophenotype profile based on the expression level of the one or more biomarkers;
(d) administering a PPAR-γ agonist to a first subset of subjects and a placebo to a second subset of subjects;
(e) repeating step (b) after the administration of the PPAR-γ agonist or the placebo; and
(f) determining if a statistically significant change in the expression level of the one or more biomarkers is seen in the first subset of subjects when compared to any change occurring in the second subset of subjects, wherein a statistically significant change in the expression level is associated with a beneficial long term clinical outcome.

20. The method of claim 19, wherein the expression level of each of from IL-6, IL-10, CRP, TNFα, FABP and PPY, and optionally the expression level of one or more of IL-7, IL-5, TNC, ICAM1, Factor VII, I309, TNFR1, A2M, TARC, eotaxin3, VCAM1, TPO, IL-18, B2M, SAA, DJ1, Aβ, tau, α-synuclein, GLP-1, PYY, insulin, HbA1c, glucose, triglycerides, HDL, LDL, vLDL, DGAT1, PPAR-γ, PPARα, and cholesterol, are measured in steps (b) and (e).

21. The method of claim 19, wherein the PPAR-γ agonist is selected from rosiglitazone and rosiglitazone XR.

22. A method of screening a subject for inclusion in an NSAID clinical trial to improve cognition or to prevent cognitive decline or dysfunction in the subject, the method comprising:
selecting the subject for screening based on one or more demographic factors or a result of one or more neurocognitive evaluations;
obtaining a blood, plasma, or serum sample from the subject;
measuring in the sample the expression level of one or more proinflammatory biomarkers selected from the group consisting of interleukin (IL)-7, tumor necrosis factor-alpha (TNFα), IL-5, IL-6, C-reactive protein (CRP), IL-10, tenascin C (TNC), soluble intracellular adhesion molecule-1 (sICAM-1), coagulation factor VII (Factor VII), I309, alpha-2 macroglobulin (A2M), chemokine (C-C motif) ligand 17 (TARC), eotaxin3, soluble vascular cell adhesion molecule-1 (sVCAM-1), thrombopoietin (TPO), fatty acid binding protein (FABP), IL-18, beta-2 microblogulin (B2M), serum amyloid A1 cluster (SAA), and pancreatic polypeptide (PPY);
determining that the subject has a proinflammatory endophenotype profile based on the expression level of the proinflammatory biomarkers; and
selecting the subject for inclusion in the NSAID clinical trial.

23. The method of claim 22, wherein the proinflammatory endophenotype profile is determined by comparing the expression level of one or more biomarkers in the blood, plasma, or serum sample obtained from the subject to the average expression level of the one or more corresponding biomarkers from a statistical sample representative of the subject.

24. The method of claim 22, further comprising one of steps (a)-(d)
(a) determining that the blood, plasma, or serum sample obtained from the subject has a statistically different expression level of each of IL-10, eotaxin 3, A2M, and SAA, and optionally Tenacin C when compared to a group of individuals in the statistical sample who do not have Alzheimer's disease; and selecting the subject for a celecoxib clinical trial;
(b) determining that the blood, plasma, or serum sample obtained from the subject has a statistically similar expression level of each of IL-10, eotaxin 3, A2M, and SAA, and optionally Tenacin C when compared to a group of individuals in the statistical sample who have Alzheimer's disease; and selecting the subject for a celecoxib clinical trial;
(c) determining that the blood, plasma, or serum sample obtained from the subject has a statistically different expression level of each of TPO, IL-5, IL-7, and I309, and optionally IL-6 when compared to a group of individuals in the statistical sample who do not have Alzheimer's disease; and selecting the subject for a naproxen clinical trial; or
(d) determining that the blood, plasma, or serum sample obtained from the subject has a statistically similar expression level of each of TPO, IL-5, IL-7, and I309, and optionally IL-6 when compared to a group of individuals in the statistical sample who have Alzheimer's disease; and selecting the subject for a naproxen clinical trial.

25. The method of claim 22, wherein the one or more demographic factors are selected from the group consisting of age, education level, and APOE ε4 allele frequency; and
the one or more neurocognitive evaluations are selected from the group consisting of a 4-point clock drawing test, a verbal fluency test, a trail making test, a list learning test, a mini-mental state exam, and combinations thereof.

26. A method of screening a subject for inclusion in a PPAR-γ agonist clinical trial to improve cognition or to prevent cognitive decline or dysfunction in the subject, the method comprising:
(a) selecting the subject for screening based on one or more demographic factors or a result of one or more neurocognitive evaluations;
(b) obtaining a blood, plasma, or serum sample from the subject;
(c) measuring in the sample the expression level of one or more biomarkers selected from the group consisting of interleukin (IL)-7, tumor necrosis factor-alpha (TNFα), IL-5, IL-6, C-reactive protein (CRP), IL-10, tenascin C (TNC), intracellular adhesion molecule-1 (ICAM1), coagulation factor VII (Factor VII), I309, tumor necrosis factor receptor-1 (TNFR1), alpha-2 macroglobulin (A2M), chemokine (C-C motif) ligand 17 (TARC), eotaxin3, vascular cell adhesion molecule-1 (VCAM1), thrombopoietin (TPO), fatty acid binding protein (FABP), IL-18, beta-2 microblogulin (B2M), serum amyloid A1 cluster (SAA), pancreatic polypeptide (PPY), Parkinson protein 7 (DJ1), beta amyloid (Aβ), tau, α-synuclein, glucagon like peptide 1 (GLP-1), peptide YY (PYY), insulin, glycated hemoglobin A1c (HbA1c), glucose, triglycerides, high density lipoprotein (HDL), low density lipoproteins (LDL and vLDLs), diacylglycerol acyl-transferase 1 (DGAT1), peroxisome proliferator-activated receptor (PPAR)-γ, PPARα, and cholesterol;
(d) determining that the subject has both a proinflammatory endophenotype profile and a metabolic endophenotype profile based on the expression level of the one or more biomarkers; and
(e) selecting the subject for inclusion in the PPAR-γ agonist clinical trial.

27. The method of claim 26, wherein the subject is determined to have both a proinflammatory endophenotype profile and a metabolic endophenotype profile by comparing the expression level of the one or more biomarkers in the plasma or serum sample obtained from the subject to the average expression level of the one or more corresponding biomarkers from a statistical sample representative of the subject.

28. The method of claim 26, wherein the expression level of each of from IL-6, IL-10, CRP, TNFα, FABP and PPY, and optionally the expression level of one or more of IL-7, IL-5, TNC, ICAM1, Factor VII, I309, TNFR1, A2M, TARC, eotaxin3, VCAM1, TPO, IL-18, B2M, SAA, DJ1, Aβ, tau, α-synuclein, GLP-1, PYY, insulin, HbA1c, glucose, triglycerides, HDL, LDL, vLDL, DGAT1, PPAR-γ, PPARα, and cholesterol, is measured in step (c).

29. The method of claim 26, wherein the PPAR-γ agonist is selected from rosiglitazone and rosiglitazone XR.

30. The method of claim 26, wherein the one or more demographic factors are selected from the group consisting of age, education level, APOE ε4 allele frequency, body mass index, waist circumference, and combinations thereof; and
the one or more neurocognitive evaluations are selected from the group consisting of a 4-point clock drawing test, a verbal fluency test, a trail making test, a list learning test, a mini-mental state exam, and combinations thereof.
